(19) United States
(12) Patent Application Publication
(10) Pub. No.: US 2019/0057134 A1
(43) Pub. Date: Feb. 21, 2019

(54) SYSTEM AND METHOD FOR AUTOMATED MICROARRAY INFORMATION CITATION ANALYSIS

(71) Applicant: Eitan Moshe Akirav, Plainview, NY (US)

(72) Inventor: Eitan Moshe Akirav, Plainview, NY (US)

(21) Appl. No.: 16/106,256

(22) Filed: Aug. 21, 2018

Related U.S. Application Data
(60) Provisional application No. 62/548,159, filed on Aug. 21, 2017.

(51) Int. Cl. G06F 17/30; G16H 50/50

(52) U.S. Cl. CPC G06F 17/30539; G16H 50/50; G06F 17/3053

(57) ABSTRACT

A method of data mining based on microarray data and a document database, comprising: receiving microarray data; generating a search of a microarray data database for information interpreting the microarray data; analyzing the microarray data based on the first search, to determine sequences of interest; receiving a topical; generating a second search of a document database for documents corresponding to the sequences of interest and a conjunction of the sequences of interest and the annotation; performing at least one quantitative comparative analysis between a first quantity of citations of the document database for documents corresponding to the sequences of interest versus a second quantity of citations for documents corresponding to a conjunction of the sequences of interest and the annotation; and ranking the sequences of interest based on the comparative quantitative analysis.

### CROSS REFERENCE TO RELATED APPLICATION

The present application is a non-provisional of, and claims benefit of priority under 35 U.S.C. § 119 from, U.S. Provisional Patent Application No. 62/548,159, filed Aug. 21, 2018, the entirety of which is expressly incorporated herein by reference.

### FIELD OF THE INVENTION

The present invention relates to citation analysis for gene chip information, and more particularly to a system and method for automated co-citation analysis for gene chip output and experimental variable(s).

### BACKGROUND OF THE INVENTION

Melissa B. Miller, and Yi-Wei Tang, “Basic Concepts of Microarrays and Potential Applications in Clinical Microbiology”, doi: 10.1128/CMR.00019-09 Clin. Microbial. Rev. October 2009 val. 22 no. 4 611-633 I October 2009, discusses DNA microarrays, also known as gene chips. A microarray is a collection of microscopic features (most commonly DNA) which can be probed with target molecules to produce either quantitative (gene expression) or qualitative (diagnostic) data. Microarrays can be distinguished based upon characteristics such as the nature of the probe, the solid-surface support used, and the specific method used for probe addressing and/or target detection. The probe refers to the DNA sequence bound to the solid-surface support in the microarray, whereas the target is the “unknown” sequence of interest. In general terms, probes are synthesized and immobilized as discrete features, or spots. Each feature contains millions of identical probes. The target is fluorescently labeled and then hybridized to the probe microarray. A successful hybridization event between the labeled target and the immobilized probe will result in an increase of fluorescence intensity over a background level, which can be measured using a fluorescent scanner. The fluorescence data can then be analyzed by a variety of methods. Experimental details including probe length and synthesis, number of possible features (i.e., density of the microarray).

Rajagopalan, D., & Agarwal, P. (2004). Inferring pathways from gene lists using a literature-derived network of biological relationships. Bioinformatics, 21(6), 788-793, is a seminal paper in the field of bioinformatics with respect to scientific literature databases.

Rajagopalan et al. discuss that increased use of high-throughput platform (omic) technologies has led to an important new problem in bioinformatics: biological interpretation of the lists of genes that are the typical output of such experiments. For example, transcriptome analysis of cell lines with and without drug treatment, results in a set of differentially expressed genes. It is important to understand whether some of these genes are functioning in a coordinated manner (a ‘pathway’). Such an interpretation of this set of genes is useful in understanding the mechanism of action of the drug. As the number of genes in such lists can often be in the hundreds, computational tools are essential to assist in the interpretation of such gene lists. One approach that has proven successful is based on quantifying the overlap of such a list of ‘interesting’ genes with a database of sets of genes associated with various biological processes (Tavazoie et al., 1999; Draghici et al., 2003; Hosack et al., 2003; Mootha et al., 2003). For example, if the gene list of interest overlaps significantly with the set of genes involved in glycolysis, one can conclude that the drug treatment experiment perturbed the glycolytic pathway. One disadvantage of such approaches is that genes must be placed in a limited number of static groups. For example, even the larger sources of pathways for signal transduction (such as BioCarta) are limited to about 300 pathways and phenomena such as cross talk are ignored. In the pathway context, another useful approach is to map the query set of interesting genes onto a set of classical pathway maps such as KEGG, BioCarta, etc. Software such as GenMAPP (Dahlquist et al., 2002) and several transcriptome analysis packages provide such capability. A hit is represented by color coding the location of the gene on the pathway map. If many genes in the query set are mapped on to a single pathway, say fatty acid metabolism, one would conclude that the drug treatment plays a role in fatty acid metabolism. Although this approach is visually pleasing, it also suffers from the somewhat artificial grouping of genes into a limited number of small pathway maps. Furthermore, this visual approach by itself provides no guidance on the statistical significance of the result.

Rajagopalan et al. proposed an alternative approach to the problem that is motivated by a systems biology perspective, and assembled a large network of biological relationships between genes and metabolites derived from various databases created by manual curation of literature. These biological relationships span many types of cellular processes including signaling, transcriptional regulation and metabolism. Given such a network and a query set of interesting genes from an omics experiment, their goal was to search the network for subnetworks consisting mostly of query genes. The set of genes in such subnetworks and the web of literature-based relationships between them will provide some biological insight into the mechanism of action. The PubGene suite of tools developed by Jenssen et al. (2001) also helps to analyze gene expression data using a literature-based network. Rajagolanan et al. present a graph-based heuristic algorithm with an associated scoring function to dynamically construct subnetworks with a high score, building on the work of Ideker et al. (2002) who developed a method to search Y2H-based protein interaction networks using a set of differentially expressed genes from a transcriptomics experiment. See, Barabasi, A.-L. and Oltvai, Z. N. (2004) Network biology: understanding the cell's functional organization. Nat. Rev. Genet., 5, 101-114; Dahlquist, K. D., Salomonis, N., Vranizan, K., Lawlor, S. C. and Conklin, B. R. (2002) Gen-MAPP, a new tool for viewing and analyzing microarray data on biological pathways. Nat. Genet., 31, 19-20; Draghici, S., Khatri, P., Martins, R. P., Ostermeier, G. C. and Krawetz, S. A. (2003) Global functional profiling of gene expression. Genomics, 81, 98-104; Hosack, D. A., Dennis, G., Jr, Sherman, B. T., Lane, H. C. and Lempicki, R. A. (2003) Identifying biological themes within lists of genes with EASE. Genome Biol., 4, R70; Ideker, T., Ozier, 0., Schwikoswki, B. and Siegel, A. F. (2002) Discovering regulatory and signalling circuits in molecular interaction networks. Bioinformatics, 18(Suppl. 1), S233-S240; Jenssen, T.-K., Leagreid, A., Komorowski, J. and Hovig, E. (2001) A literature network of human genes for high- throughput analysis of gene expression. Nat. Genet., 28, 21-28; Matys, V., Fricke, E., Geffers, R., Gossling, E., Haubrock, M., Hehl, R., Hornischer, K., Karas, D., Kel, A. E., Kel-Margoulis, O. V. et al. (2003) Transfac: transcriptional regulation, from patterns to profiles. Nucleic Acids Res., 31, 374-378; Mootha, V., Lindgren, C., Eriksson, K., Subramanian, A., Sihag, S., Lehar, J., Puigserver, P., Carlsson, E., Ridderstrale, M., Laurila, E. et al. (2003) PGC-1 alpha responsive genes involved in oxidative phosphorylation are coordinately downregulated in human diabetes. Nat. Genet., 34, 267-273; Tavazoie, S., Hughes, J. D., Campbell, M. J., Cho, R. J. and Church, G. M. (1999) Systematic determination of genetic network architechture. Nat. Genet., 22(3), 281-285.

Philip Zimmermann, Lars Hennig and Wilhelm Gruissem, “Gene-expression analysis and network discovery using Genevestigator”, discusses the Genevestigator software suite, a web-based tool that provides categorized quantitative information about elements (genes or annotations) contained in large microarray databases. The identification of gene function is the main task of functional genomics and molecular biology. Several data repositories exist that accumulate and classify the constantly increasing amount of microarray data, and sophisticated software tools enable the analysis of individual experiments after data are downloaded. By contrast, few web-based applications provide an easy-to-use and biological context- oriented querying of large gene-expression databases.

Grimes, G R, Wen, T Q, Mewissen, M, Baxter, R M, Moodie, S, Beattie, JS & Ghazal, P 2006, ‘PDQ Wizard: automated prioritization and characterization of gene and protein lists using biomedical literature’ Bioinformatics, vol 22, no. 16, pp. 2055-7. DOI: 10.1093/bioinformatics/bt1342, discloses PDQ Wizard, software which automates the process of interrogating biomedical references using large lists of genes, proteins or free text. Using the principle of linkage through co-citation, biologists can mine PubMed with these proteins or genes to identify relationships within a biological field of interest. PDQ Wizard provides features to define more specific relationships, highlight key publications describing those activities and relationships, and enhance protein queries. PDQ Wizard also outputs a metric that can be used for prioritization of genes and proteins for further research. This prioritization weights multiplicity of citation as a positive ranking factor.

High-throughput technologies are widely used for the global and parallel measurement of gene and protein activity within biological systems. A primary output from these analyses is often a collection of tens or hundreds of genes or proteins of interest. A major challenge for biologists, therefore, is to rapidly derive comprehensive information about the biological processes for each of the specific genes or proteins in the list and to identify where domain-specific relationships exist. Several databases, such as Entrez Gene (Maglott et al., 2005) and UniProt (Bairoch et al., 2005) enable biologists to access information on individual genes and proteins. Biologists, however, frequently require more in-depth, specific information than is included in these databases and need to be able to explore gene and protein lists rather than individual identifiers.

The detailed information biologists require is primarily stored as free text within large biomedical literature databases such as PubMed (Wheeler et al., 2005). Significantly, Entrez (Wheeler et al., 2005) which is the main interface for searching and retrieving information from PubMed, is not designed for searching with multiple gene or protein identifiers, such as Entrez Gene Ids. Consequently, it is inadequate for the rapid interrogation of literature relating to multiple genes and proteins.

Several tools, such as microGenie (Korotkiy et al., 2004) and MILANO (Rubinstein and Simon, 2005) have been developed to automate the annotation, batch query and data retrieval steps during PubMed searches. These gene-based search applications are limited to providing a single method to identify co-citation relationships, and they are restricted from further refinement of results or alternative querying strategies and do not permit the use of protein identifiers. PDQ Wizard provides a system that identifies relationships between lists of gene or protein identifiers and user defined terms based on their co-occurrence within PubMed literature references. The system outputs a table that includes the original gene or protein identifiers, with associated information such as the gene synonyms, gene description and the list of user defined terms. For each gene/protein Id and user defined term pair the number of PubMed records co-citing these terms are also displayed. PDQ Wizard provides several features including the following: Interactive filtering of results, giving the ability to refine pairwise relationships and metrics for prioritization; Identification of top publications for a list of genes or proteins; Provides a view of publication information, including title and abstract, with syntax highlighting, similar to PubMed; Protein identifier input, providing support for Swiss-Prot identifiers. Using PDQ Wizard, the user enters a list of genes or proteins alongside a set of keyword terms. PDQ automatically annotates lists, generates PubMed queries and retrieves results. The results are presented as a table showing the number of co-citations for gene/protein identifier and user defined term pairs. The user has the choice of (1) Filtering results, (2) examining the references and (3) identifying publications that are present in multiple hits.

To cope with the multiplicity in biological naming, PDQ Wizard utilizes a gene and protein thesaurus derived from information stored within the UniProt and Entrez Gene databases. This is used to annotate identifiers with their corresponding official gene symbols, protein names, gene descriptions and synonyms. These annotations are automatically combined with user defined terms to construct enhanced PubMed queries. To limit the number of results retrieved due to synonymous terms within the literature, the thesaurus is filtered to remove gene/protein synonyms that match words found within an English dictionary, biological acronyms and biological abbreviations. Gene names are not subject to filtering, however, they must match the exact phrase for a search to retrieve results. For example, for the Drosophila gene ‘bag of marbles’ the entire gene name must appear in the publication to classify as a hit.

In a typical example, a biologist inputs a list of differentially regulated genes from a microarray experiment alongside a number of terms. These user defined terms are normally related to the biologist's field of scientific interest or the experimental system the lists are derived from. For example, for a list of differentially regulated genes derived from a microarray experiment where cells had been treated with interferon, a biologist may enter the term ‘interferon’. Next PDQ Wizard queries PubMed and presents the results as a table of the pairwise co-occurrence of each gene or protein identifier and user defined term within PubMed. A ‘hit’ between an identifier and keyword indicates that both terms are co- cited within a PubMed record and may have an underlying relationship. Therefore, the user can use the finding of hits to categorize their list according to the relationship with keyword terms. The greater the number of hits, the more likely the inferred association (Marcotte and Date, 2001). As a result, biologists can use the number of hits to prioritize their future literature research based on the most likely gene/protein and user defined term relationships within their field of interest. Biologists wishing to further categorize their lists can use the filter toolbar to input additional terms. The filter toolbar appends additional terms to the query table using the ‘AND’ operator. Users can also restrict these searches to specific fields within a PubMed record, e.g. title. For example, if an initial search has identified a subset of genes that have a relationship with ‘interferon’, a user may enter the term ‘JAK’ in the filter toolbar to identify which of those genes are related to the JAK pathway. The results then show the table of hits for the gene list, ‘interferon’ and ‘JAK’, which can then be used to re- classify the gene list. Another key task biologists perform is to identify publications that describe the relationship between multiple members of their gene or protein lists. PDQ Wizard provides the option to identify these key publications in the results using the ‘top publication’ feature. A top publication is defined as one that appears in multiple hits, so it should contain information that links multiple members of the gene or protein list with the user defined terms. This feature is especially useful for identifying those publications that describe biological pathways.

PDQ Wizard is implemented as a Java Server Faces web application utilizing Apache Tomcat as the web server. The component that provides access to the PubMed server works through the Entrez utilities web service (Wheeler et al., 2005). The PubMed web service imposes limitations on its usage; this includes a maximum of one query every 3 seconds (Korotkiy et al., 2004). Therefore, to perform a search using 10 gene/protein identifiers and 10 user defined terms or 100 queries would take about 5 min. The gene/protein thesaurus is stored within a MySQL database that contains gene and protein annotations parsed from Entrez Gene and UniProt database files using custom Python scripts. PubMed abstracts downloaded for manual inspection are cached locally to increase response time and reduce the load on the PubMed server.

PDQ Wizard is a web-based tool that enables the rapid classification and prioritization of large lists of gene and protein identifiers using the biomedical literature. The classification is based on the presence of genes or proteins and user defined terms within the literature, and the prioritization is based on the number of literature references retrieved for each identifier and user defined term pair. The system also provides novel features to further classify results, highlight relevant publications and manually inspect literature references. See, Bairoch, A. et al. (2005) The Universal Protein Resource (UniProt). Nucleic Acids Res., 33, D154-159; Korotkiy, M. et al. (2004) A tool for gene expression based PubMed search through combining data sources. Bioinformatics, 20, 1980-1982; Maglott, D. et al. (2005) Entrez Gene: gene-centered information at NCBI. Nucleic Acids Res., 33, D54-58; Marcotte, E. and Date, S. (2001) Exploiting big biology: integrating large-scale biological data for function inference. Brief Bioinform., 2, 363-374; Pearson, H. (2001) Biology's name game. Nature, 411, 631-632; Rubinstein, R. and Simon, I., (2005) MILANO-custom annotation of microarray results using automatic literature searches. BMC Bioinformatics, 6, 12; Wheeler, D.L. et al. (2005) Database resources of the National Center for Biotechnology Information. Nucleic Acids Res., 33, D39-D45.

M. Ghanem, Y. Guo and A.S. Rowe, “Integrated Data Mining and Text Mining In Support of Bioinformatics”, discloses a Discovery Net, a bioinformatics data mining scheme. A plethora of online database sources provides curated background information in the form of structured (data tables) and semi-structured (such as XML) content about genes, their products and their involvement in identified biological systems. However, the main source of most background knowledge still remains to be scientific publication databases (e.g. Medline) that store the available information in an unstructured form; the required information is embedded within the free text found in each publication.

As a first example, a scientist may be engaged in the analysis of microarray gene expression data using traditional data clustering techniques. The result of this clustering analysis could be a group of co-regulated genes (i.e. genes that exhibit similar experimental behavior) or could be groups of differentially expressed genes. Once theses groupings are isolated, the scientist may wish to investigate and validate the significance of his findings by: Seeking background information on why such genes are co-regulated or differentially expressed, and identifying the diseases that are associated with the different isolated gene groupings. Much of the required information is available on online genomic databases, and also in scientific publications. The Discovery Net workflow is divided into three logical phases. The first phase (“Gene Expression Analysis”), corresponds to the traditional data mining phase, where the biologist conducts analysis over gene expression data using a data clustering analysis component to find co-regulated/differentially expressed genes. The output of this stage is a set of “interesting genes” or “gene groupings” that the data clustering methods isolate as being candidates for further analysis. In the second phase of the workflow (“Find Relevant Genes from Online Databases”) the user uses the InfoGrid integration framework to obtain further information about the isolated genes from online databases. In this phase, the workflow starts by obtaining the nucleotide sequence for each gene by issuing a query to the NCBI database based on the gene accession number. The retrieved sequence is then used to execute a BLAST query to retrieve a set of homologous sequences; these sequences in turn are used to issue a query to the SwissProt database to retrieve the PubMed Ids identifying articles relating to the homologous sequences. Finally, the PubMed Ids are used to issue a query against PubMed to retrieve the abstracts associated with these articles, and the abstracts are passed through a frequent phrase identification algorithm to extract summaries for the retrieved documents for the gene and its homologues. Finally, in the third phase of the workflow (“Find Association between Frequent Terms”) the user uses a dictionary of disease terms obtained from the MESH (Medical Subject Headings) dictionary to isolate the key disease terms appearing in the retrieved articles. The identified disease words are then analyzed using a standard association analysis a priori style algorithm to find frequently co-occurring disease terms in the retrieved article sets that are associated with both the identified genes as well as their homologues.

The second example shows how the Discovery Net infrastructure can support finding correlations between data sets obtained from different experiments. In this case, these are two data sets, one obtained from microarray experiments and the other from NMR-based metabonomic experiments. Both data sets are obtained from a project relating to studying insulin resistance in mice. The microarray gene expression data measures the amount of RNA expressed at the time a sample is taken, and the NMR spectra are for metabolites found in urine samples of the same subjects. In this example, the user is interested to find known associations between the genes that isolated as “interesting” from the first data set and the metabolites identified as “interesting” from the second. This analysis proceeds into three logical phases: The first phase (“Microarray analysis) uses standard gene expression analysis technique to filter interesting genes within the gene expression domain. The gene expression process that is used is starts by mapping the gene expression probe id to the sequence that would bind to that area. Using the sequence, BlastX is used to search the Swiss-Prot database. This provides a method of finding known genes. After the blast process, the hits from this database are used to download features from the actual records from the Swiss-Prot database to annotate the probe ID with possible gene names for the sequence and any Enzyme commission number when it exists. In parallel, the second phase (“Metabonomic Analysis”) proceeds by analysis the NMR data using multivariate analysis to study the NMR shifts, and mapping them to candidate metabolites using both manual processes and NMR shift databases. The output of this phase is a set of candidate metabolite names. The third phase (“Text Selections and Relationship Functions”) then proceeds based on the “joining” the outputs of the phases 1 and 2 to find known associations between the genes and the metabolites. This phase proceeds by a) Searching pathway databases for known relationships between the metabolites and the genes, and b) Searching scientific publications using a co-occurrence analysis approach to find the most general relationships possible between the metabolites and the genes. The outputs of both types of analysis is then merged and presented to the user. See, V. Curcin, M. Ghanem, Y. Guo, M. Kohler, A. Rowe, J Syed, P. Wendel. Discovery Net: Towards a Grid of Knowledge Discovery. Proceedings of KDD-2002. The Eighth ACM SIGKDD International Conference on Knowledge Discovery and Data Mining. Jul. 23-26, 2002 Edmonton, Canada; Giannadakis N, Rowe A, Ghanem M and Guo Y. InfoGrid: Providing Information Integration for Knowledge Discovery. Information Science, 2003: 3: 199-226; Rowe A, Ghanem M, Guo Y. Using Domain Mapping to Integrate Biological and Chemical Databases. International Chemical Information Conference, Nimes, 2003; Ghanem M. M, Guo Y, Lodhi H, Zhang Y, Automatic Scientific Text Classification Using Local Patterns: KDD CUP 2002 (Task 1), SIGKDD Explorations, 2002. Volume 4, Issue 2.

Min Song, SuYeon Kim, Guo Zhang, Ying Ding, Tamy Chambers, “Productivity and Influence in Bioinformatics: A Bibliometric Analysis using PubMed Central” manuscript (2013), discuss the use of bioinformatics, based on the optimal the use of “big data” gathered in genomic, proteomics, and functional genomics research. The paper looks to popularity and citation counts as a factor in favor of importance.

Ding, Y., Zhang, G., Chambers, T., Song, M., Wang, X., & Zhai, C. (2014). Content-based citation analysis: The next generation of citation analysis. Journal of the Association for Information Science and Technology, 65(9), 1820-1833, discuss that traditional citation analysis has been widely applied to detect patterns of scientific collaboration, map the landscapes of scholarly disciplines, assess the impact of research outputs, and observe knowledge transfer across domains. It is, however limited, as it assumes all citations are of similar value and weights each equally. Content-based citation analysis (CCA) addresses a citation's value by interpreting each based on their contexts at both syntactic and semantic level.

Dennise D. Dalma-Weiszhausz, Janet Warrington, Eugene Y. Tanimoto, and C. Garrett Miyada, “The Affymetrix GeneChip Platform: An Overview”, Methods In Enzymology, Vol. 410 (2006) discusses the Affymetrix GeneChip system. Gene expression profiling studies are performed with the goal of comparing tissues, tissue types, and cellular responses to a variety of stimuli such as altered growth conditions, cancer, and infectious processes to gain biological insight into basic biochemical pathways or molecular mechanisms of disease and its regulatory circuits. Whole-genome expression analysis has already helped scientists stratify disease, predict patient outcome, compare strains with varying virulence, study the relationship between host and parasite, and understand the affected molecular pathways of certain diseases. The volume of publications in this field is immense, resulting in information overload.

Genomatix, www.genomatix.de, provides various software tools for genetic information analysis. GeneRanker is a program allowing characterization of large sets of genes by making use of annotation data from various sources, like Gene Ontology or Genomatix proprietary annotation. Overrepresentation of different biological terms within the input are calculated and listed in the output together with the respective p-value. The algorithm behind GeneRanker is based on the paper Gabriel F. Berriz et al. (2003), “Characterizing gene sets with FuncAssociate”, Bioinformatics 19, 2502-2504 (PubMed: 14668247). LitInspector is a literature search tool for automatic gene and signal transduction pathway data mining within the NCBI PubMed database. LitInspector allows input of gene synonyms or gene IDs and free text. The query can be filtered for only those abstracts for which also defined keyword categories (tissue, disease, pathway, or small molecule) were identified. See, Frisch M, Klocke B, Haltmeier M, Frech K (2009), “LitInspector: literature and signal transduction pathway mining in PubMed abstracts”, Nucleic Acids Res. PUBMED: 19417065, nar.oxfordjournals.org/cgi/content/full/gkp303. See also Liu, H., & Rastegar-Mojarad, M. (2016). Literature-based knowledge discovery. Big Data Analysis for Bioinformatics and Biomedical Discoveries, 233-248; Jung, J. Y., DeLuca, T. F., Nelson, T. H., & Wall, D. P. (2013). A literature search tool for intelligent extraction of disease-associated genes. Journal of the American Medical Informatics Association, 21(3), 399-405; Patnala, R., Clements, J., & Batra, J. (2013). Candidate gene association studies: a comprehensive guide to useful in silica tools. BMC genetics, 14(1), 39; Coassin, S., Brandstatter, A., & Kronenberg, F. (2010). Lost in the space of bioinformatic tools: a constantly updated survival guide for genetic epidemiology. The GenEpi Toolbox. Atherosclerosis, 209(2), 321-335; Sreekala, S., & Nazeer, K. A. (2014, December). A literature search tool for identifying disease-associated genes using Hidden Markov model. In Computational Systems and Communications (ICCSC), 2014 First International Conference on (pp. 90-94). IEEE; Wu, C., Schwartz, J. M., & Nenadic, G. (2013). PathNER: a tool for systematic identification of biological pathway mentions in the literature. BMC systems biology, 7(3), S2; Li, C., Liakata, M., & Rebholz-Schuhmann, D. (2013). Biological network extraction from scientific literature: state of the art and challenges. Briefings in bioinformatics, 15(5), 856-877; Qiao, N., Huang, Y., Naveed, H., Green, C. D., & Han, J. D. J. (2013). CoCiter: an efficient tool to infer gene function by assessing the significance of literature co-citation. PLoS One, 8(9), e74074.

Various patents discuss citation analysis, which provide context and embodiments usable with or in accordance with the present technology: 5,544,352; 5,594,897; 5,832,494; 5,870,770; 5,930,784; 5,966,126; 5,987,470; 6,038,574; 6,098,064; 6,112,202; 6,175,824; 6,182,091; 6,233,571; 6,256,648; 6,263,351; 6,285,999; 6,286,018; 6,289,342; 6,326,962; 6,385,611; 6,385,629; 6,389,436; 6,415,282; 6,457,028; 6,505,197; 6,519,602; 6,539,376; 6,549,896; 6,556,992; 6,560,600; 6,604,114; 6,651,058; 6,651,059; 6,665,656; 6,665,670; 6,675,170; 6,684,205; 6,728,725; 6,738,780; 6,799,176; 6,856,988; 6,871,202; 6,882,992; 6,886,129; 6,952,806; 6,970,103; 7,038,680; 7,058,628; 7,062,498; 7,243,109; 7,433,884; 7,552,398; 7,668,787; 7,734,624; 7,809,705; 7,117,198; 7,243,130; 7,444,383; 7,565,403; 7,668,825; 7,743,340; 7,818,279; 7,130,848; 7,246,310; 7,457,879; 7,580,939; 7,672,950; 7,752,208; 7,822,774; 7,136,875; 7,269,587; 7,464,025; 7,624,081; 7,676,375; 7,778,954; 7,840,524; 7,139,752; 7,296,016; 7,493,320; 7,634,528; 7,693,704; 7,783,592; 7,844,449; 7,146,361; 7,302,638; 7,512,602; 7,647,335; 7,707,210; 7,783,619; 7,844,666; 7,162,508; 7,333,984; 7,526,475; 7,647,345; 7,716,060; 7,783,668; 7,908,277; 7,213,198; 7,391,885; 7,529,756; 7,653,608; 7,716,226; 7,788,264; 7,930,295; 7,233,943; 7,400,981; 7,548,917; 7,657,507; 7,734,567; 7,792,827; 7,933,843; 7,937,405; 7,953,724; 7,962,511; 7,966,328; 7,970,773; 7,975,015; 7,975,301; 7,987,198; 8,001,157; 8,010,482; 8,010,646; 8,019,834; 8,024,415; 8,032,820; 8,073,838; 8,086,523; 8,086,672; 8,095,876; 8,126,882; 8,126,884; 8,131,701; 8,131,715; 8,131,717; 8,135,662; 8,145,617; 8,145,675; 8,150,842; 8,166,061; 8,170,971; 8,176,440; 8,185,530; 8,195,651; 8,204,852; 8,230,364; 8,239,372; 8,250,118; 8,260,789; 8,280,903; 8,280,918; 8,291,492; 8,306,987; 8,316,001; 8,316,292; 8,332,418; 8,335,785; 8,347,237; 8,370,359; 8,392,349; 8,407,139; 8,458,185; 8,473,487; 8,479,091; 8,489,630; 8,494,897; 8,495,099; 8,504,551; 8,504,560; 8,504,586; 8,515,893; 8,515,937; 8,516,357; 8,521,730; 8,522,129; 8,527,442; 8,555,196; 8,566,360; 8,566,413; 8,577,831; 8,583,592; 8,583,658; 8,589,784; 8,595,204; 8,600,974; 8,612,411; 8,630,975; 8,635,281; 8,639,695; 8,645,396; 8,661,033; 8,661,066; 8,662,279; 8,671,102; 8,683,389; 8,684,158; 8,694,419; 8,700,738; 8,701,027; 8,719,005; 8,725,726; 8,732,101; 8,756,187; 8,768,911; 8,782,050; 8,799,237; 8,799,952; 8,805,781; 8,805,814; 8,818,996; 8,819,000; 8,832,002; 8,843,519; 8,909,583; 8,930,304; 8,935,291; 8,938,458; 8,972,875; 8,983,965; 8,990,124; 9,009,088; 9,037,615; 9,053,179; 9,069,853; 9,075,849; 9,075,873; 9,087,129; 9,098,573; 9,135,331; 9,152,718; 9,165,040; 9,171,338; 9,176,938; 9,177,050; 9,177,249; 9,177,349; 9,183,290; 9,195,962; 9,196,097; 9,201,969; 9,208,443; 9,218,344; 9,251,433; 9,251,434; 9,262,514; 9,262,526; 9,262,749; 9,264,329; 9,268,821; 9,268,849; 9,269,051; 9,289,374; 9,305,215; 9,311,360; 9,336,330; 9,348,919; 9,367,604; 9,369,765; 9,442,986; 9,443,004; 9,443,022; 9,449,336; 9,460,475; 9,461,876; 9,471,672; 9,483,472; 9,524,498; 9,542,622; 9,552,420; 9,558,265; 9,588,955; 9,594,809; 9,613,321; 9,646,082; 9,697,506; 9,723,059; RE43753; 20020035499; 20020062302; 20020103818; 20020178136; 20020194018; 20030128212; 20030130994; 20030172020; 20040015481; 20040049503; 20040093327; 20040111412; 20040122841; 20040128273; 20040243554; 20040243556; 20040243557; 20040243560; 20040243645; 20050071310; 20050071311; 20050071743; 20050138056; 20050144169; 20050149523; 20050149524; 20050165736; 20050165757; 20050165780; 20060106847; 20060112111; 20060149720; 20060184464; 20060259455; 20060282380; 20070288442; 20080133585; 20070050393; 20070299547; 20080195631; 20070073748; 20070299872; 20080215563; 20070112763; 20070300170; 20080256093; 20070239431; 20070300190; 20080270314; 20070266144; 20080033929; 20080270395; 20080270446; 20080275859; 20080306934; 20090043797; 20090070297; 20090070366; 20090083314; 20090132901; 20090157585; 20090222441; 20090234829; 20090254543; 20100030749; 20100106752; 20100145956; 20100185513; 20100217731; 20100241947; 20100312764; 20100332520; 20110016115; 20110016134; 20110066714; 20110072024; 20110153613; 20110161089; 20110173191; 20110173264; 20110177966; 20110191309; 20110246578; 20110264672; 20110282890; 20110295903; 20120011156; 20120078876; 20120123974; 20120197904; 20120221580; 20120233152; 20120323880; 20130080266; 20130090984; 20130144875; 20130204671; 20130232263; 20140040027; 20140046962; 20140067829; 20140075004; 20140101557; 20140108273; 20140156544; 20140161360; 20140161362; 20140188780; 20140195539; 20140214825; 20140258146; 20140258147; 20140258148; 20140258149; 20140258150; 20140258151; 20140258153; 20140324711; 20150026105; 20150046420; 20150072356; 20150135222; 20150161256; 20150169559; 20150169758; 20150186789; 20150205869; 20150233930; 20150306022; 20150310000; 20160004768; 20160019231; 20160042054; 20160048556; 20160098407; 20160110447; 20160166626; 20160170814; 20160171391; 20160196332; 20160203256; 20160224622; 20160335257; 20160344828; 20160371598; 20170039297; 20170060983; 20170076219; 20170132314; 20170235819; and 20170235848.

All references and patents disclosed herein are expressly incorporated herein by reference in their entirety, for all purposes.

### SUMMARY OF THE INVENTION

Recent technology allows for the analysis of the biological difference between treatment condition by comparing cells, tissues, or whole organisms. The output of these techniques includes protein and gene of hundreds, thousands and sometimes tens of thousands candidates. The National Institute of Health public repository provides access to hundreds of gene arrays ready for data mining. Currently, several techniques exist for prioritization of gene candidates including pathway analysis. While useful, these are affected by user biases and in many cases have limited information.

The present technology provides a system and method for performing automated citation lookup and ranking/prioritization based on co-citation of genes identified in a microarray output, and another search term (i.e., an experimental variable), seeking to determine, e.g., understudied genes for which a body of literature exists, e.g., in other fields.

This technology generally differs from prior techniques in that it emphasizes those results that are rare, over those with a higher citation count. As a result, the output can be a list of leads for further research where fundamental investigation may be lacking, and therefore significant unknown remain. This technology therefore seeks “questions” and not “answers”, and in this way fundamentally differs from more typical citation analysis, where one seeks explanations, confirmation, or related work to the data provided by the researcher.

In operation, results from a microarray experiment, e.g., a GeneChip, are provided, e.g., as a spreadsheet or other tabulated data in standardized form.

The present technology provides a way by which DNA construct prioritization is done automatically, by selecting cross referencing gene array data and the desired keyword(s) against the number of citations available for the gene and the keyword(s), and the total number of citation available for the specific gene. A ratio between the keyword(s) plus gene, vs. the total citation number of the gene is then computed. A high ratio suggests that this gene is well studied in a given discipline (keyword) and a low ratio suggests that this gene is well studied generally but less so in a given discipline. This is an objective prioritization method to provide researchers with information on the popularity of the gene in the experimental system in a given field. An embodiment of the invention is provided on GitHub github.com/BioDataSorter/BioDataSorter.

The technology may also apply journal impact factor, a whitelist or a blacklist as a filter, and journal impact factor, forward citations, co-citations, author citations, or other metadata or citation factors in modifying the output of the Medline search, or use in place a Google Scholar search or other database. In many cases, applying such constraints requires a very complex search query, or a large number of queries, or both. For example, a researcher may seek to exclude “low quality” journals from the analysis. For example, a whitelist or blacklist of journal names may be applied to exclude predatory journals. On the other hand, separate metrics may be produced for high quality and low quality publications, which may reveal biases. Journal impact factor may also by applied, but unless supported as a basic feature of the database, requires separate citation metrics for each journal, which can then be weighted. Typically, high impact factor and high quality journals are favorable factors in a ranking. However, according to one aspect of the technology, the sparsity of citation metric as a heuristic for understudied genes for particular diseases may be modified to consider non-mainstream research of genes associated with keywords or conditions. In this case, a skew of distribution of a gene or set of genes toward low impact journals may be a factor in favor of potentially impactful future research in the field, though with a warning that the existing research is not published in the high impact journals. On the other hand, if consideration is limited to high quality, high impact journals only, the “noise” resulting from low quality journals is minimized, perhaps leading to a better analysis of the potential for future research in a field. Thus, these factors may be added to the search, analysis and presentation strategy, with either a predetermined effect on the output, or as a set of user-selectable options.

The Medline/PubMed database does not provide full text searching. Therefore, given typical policies for article titles, abstracts, and keywords, the sematic content of these records is well curated. On the other hand, these fields are all populated prospectively, and may exclude data of interest retrospectively. The Google Scholar database, which has some different coverage from PubMed, typically provides full text indexing. Therefore, when searching for gene occurrences in the literature, Google Scholar or other full text resources will yield distinct results. Therefore, another aspect of the technology is to automate searching and analysis of a full text database resource, which in some cases may require downloading of articles to complete the automated analysis. Further, comparing full text vs. abstract record results may provide useful insights. Similarly, a search on either type of database may be date limited, and temporally segmented, to provide indication of trends. Gene mentions of increasing popularity probably indicate that new research on the same or similar topic will be duplicative or cumulative, especially given the lag between starting new research and publication.

A further aspect of the technology is conducting searches for multiple concurrent gene mentions. That is, some genes may be both important and common. However, by searching the conjunction of multiple genes, a more fine-grained output can be achieved. This is physiologically sound, since correlated changes in microarray data often reflect underlying linkages between genes and gene biology. Accordingly, instead of performing a search for each gene with potential significance, combinations of 2 or more genes may be searched, to produce joint citation indices. Further, in some cases, important information is revealed by a lack of significant change in a gene (which may be coupled to significance of another gene. Such combinatorial searching may require hundreds or thousands of individual queries, or mass downloading of abstracts or references any local analysis.

Therefore, the technology is not limited to seeking a simple co-citation of a gene and a keyword, and may include various complex, iterative, and multi-database searching.

The technology is also not limited to genetic or microarray data, and may be applied in various cases where exploration of large data sets require initial screening of the data according to a heuristic such as citation counts, with a preferred paradigm being to seek understudied issues by looking for large ratios of total citations vs. topic-specific citations, in view of data which at least hints at a likely relation worthy of further investigation.

It is therefore an object to provide a method of data mining based on microarray data database and a document database, comprising: receiving microarray data; generating a first search of a microarray data database for information for interpreting the microarray data; determining sequences of interest of the microarray data based on results of the first search; receiving a topical annotation; generating a second set of searches of a document database for documents corresponding to the sequences of interest, and a conjunction of the sequences of interest and the annotation; performing at least one comparative quantitative analysis between a first quantity of citations of the document database for documents corresponding to the sequences of interest versus a second quantity of citations for documents corresponding to a conjunction of the sequences of interest and the annotation; and ranking the sequences of interest based on the comparative quantitative analysis.

It is also an object to provide a system for data mining based on microarray data database and a document database, comprising: an input port configured to receive microarray data; a communication network interface port; at least one processor, configured to: generate a first search of a microarray data database for information for interpreting the microarray data; conduct the first search on the microarray data database through the communication network interface port; determine sequences of interest of the microarray data based on results of the first search; receive a topical annotation; generate a second set of searches for a document database for documents corresponding to the sequences of interest, and a conjunction of the sequences of interest and the annotation; conduct the second search on the document data database through the communication network interface port; perform at least one comparative quantitative analysis between a first quantity of citations of the document database for documents corresponding to the sequences of interest versus a second quantity of citations for documents corresponding to a conjunction of the sequences of interest and the annotation; and rank the sequences of interest based on the comparative quantitative analysis; and an output port configured to present the ranked sequences.

It is a further object to provide a computer readable medium storing thereon nontransitory instructions for causing an automated data processing system to perform the steps of: generating a first search of a microarray data database for information for interpreting a set of microarray data; conducting the first search on the microarray data database through a communication network interface; determining sequences of interest of the microarray data based on results of the first search; receiving a topical annotation; generating a second set of searches for a document database for documents corresponding to the sequences of interest, and a conjunction of the sequences of interest and the annotation; conducting the second search on the document data database through the communication network interface; performing at least one comparative quantitative analysis between a first quantity of citations of the document database for documents corresponding to the sequences of interest versus a second quantity of citations for documents corresponding to a conjunction of the sequences of interest and the annotation; and ranking the sequences of interest based on the comparative quantitative analysis.

A sequence of interest having a high ratio of the first quantity of citations to the second quantity of citations may be ranked higher than a sequence of interest having a low ratio of the first quantity of citations to the second quantity of citations.

The ranking based on the comparative quantitative analysis may be presented as a word cloud. Sequences of interest for which the first quantity of references is below a threshold number may be excluded from the ranking.

The microarray data database may comprise the NCBI GEO database. The document database may comprise the NCBI Pubmed database. The microarray data database and/or the document database may be accessed through the Internet.

### BRIEF DESCRIPTION OF THE DRAWINGS

FIG. 1 shows an exemplary “word cloud” according to the present invention.

FIG. 2 shows an NCBI GEO database search page with results for “diabetes”.

FIG. 3 shows an NCBI GEO database statistical analysis page.

FIG. 4 shows an NCBI GEO database output sort page.

FIG. 5 shows a BioDataSorter software interface screen.

FIGS. 6 and 7 show an NCBI PubMed input search page, showing a search for gene name+gene symbol (FIG. 6) and a search for gene name+gene symbol+keyword (FIG. 7).

### DETAILED DESCRIPTION OF THE PREFERRED EMBODIMENTS

A preferred embodiment of the technology executes computer instructions to control a general purpose computer to execute a set of logic. The computer instructions may be stored on a non-transitory computer readable medium.

The program, for example, takes data in the form of a Microsoft Excel® spreadsheet that has a gene “Symbol” column and “Synonyms” column, similar to spreadsheets that can be downloaded from NCBI's Gene Expression Omnibus (GEO), which is a public functional genomics data repository for array-based and sequence-based data.

The NCBI GEO Data may be obtained manually or automatically. A keyword is searched in GEO, and the datasets results selected. Particular results may be manually selected. The option “Compare 2 sets of samples” may be selected, and sample groups chosen to analyze gene fluctuations. The link provided leads to the profile data results, and up to 500 items per page may be obtained. The profile data may then be downloaded, and converted to a text file or Microsoft Excel® document (.xlsx)

The preferred automation software BioDataSorter is implemented in Python 3, and employs Biopython (biopython.org/). The BioDataSorter receives as an input the downloaded spreadsheet from NCBI GEO.

The program is designed to sort gene array data from GEO or another repository as follows:
1. Data is sorted in an excel sheet with gene name and gene symbol labelled on the top of the relevant column.
2. The user can limit the list to those genes that are statistically significant between the experimental groups.
3. Gene name(s)+gene symbols are sent to the search box at www.ncbi.nlm.nih.gov/pubmed/(the US National Library of Medicine).
4. Total Number of citations is then reported back to the app and placed in a newly generated column in the excel sheet “Total Citation.” In addition, a description of the gene, if available, is downloaded from PubMed, and inserted into a column in the spreadsheet. This facilitates user analysis, since the description, if available, can be observed by “hovering” a cursor over the cell, and passed on for further analysis or presentation.
5. A second search which includes Gene name(s)+gene symbol+Keyword are sent to www.ncbi.nlm.nih.gov/pubmed/ the US National Library of Medicine. The key word is chosen based on the field of interest/hypothesis tested. See FIG. 6.
6. The number of citation limited by key word is reported back to the Excel sheet.
7. The number of citation generated by the keyword is divided by the total number of citations for the given gene. See, FIG. 7.
8.Ratio is reported in a “Ratio Column.”
9. The excel sheet is saved as the output file.

10. Top ratios are presented as a word cloud output for visualization purposes.

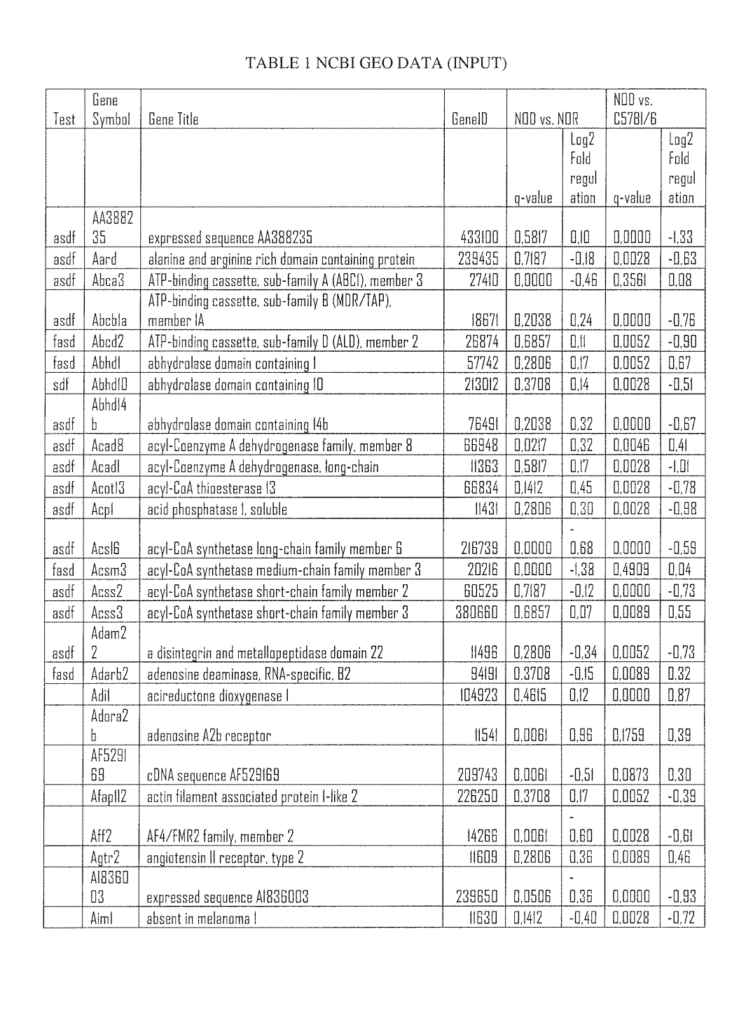

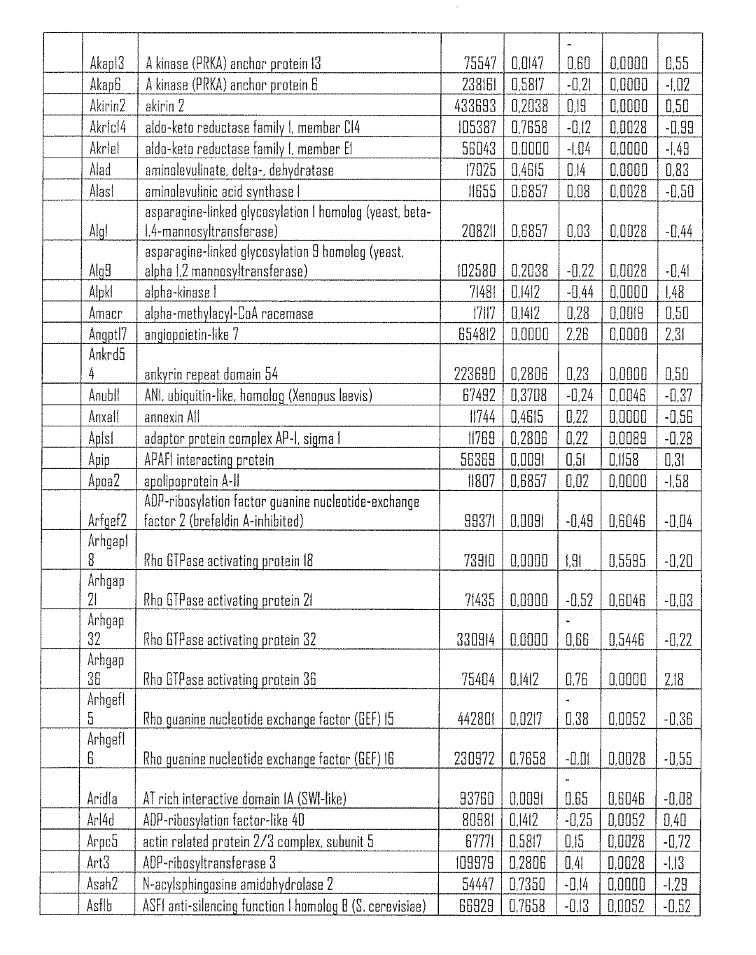

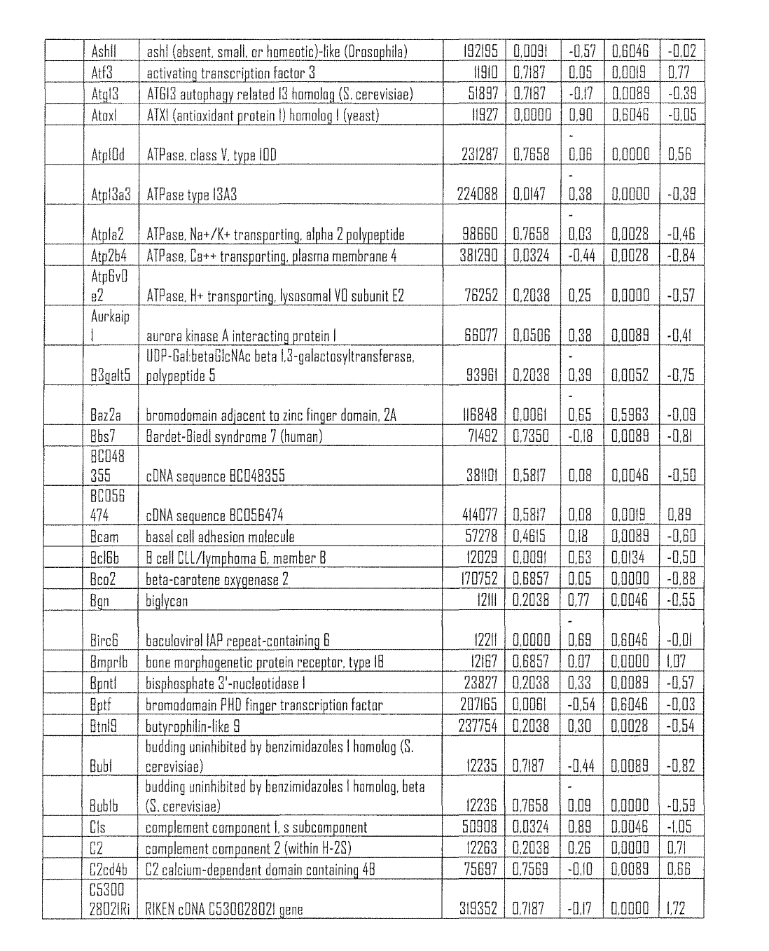

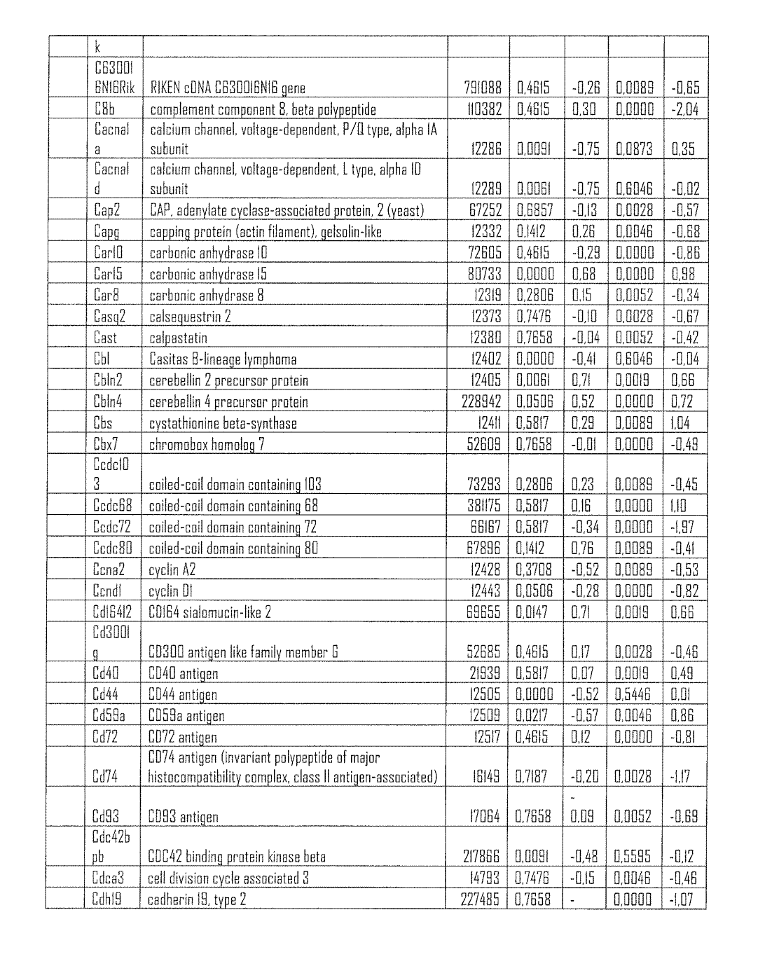

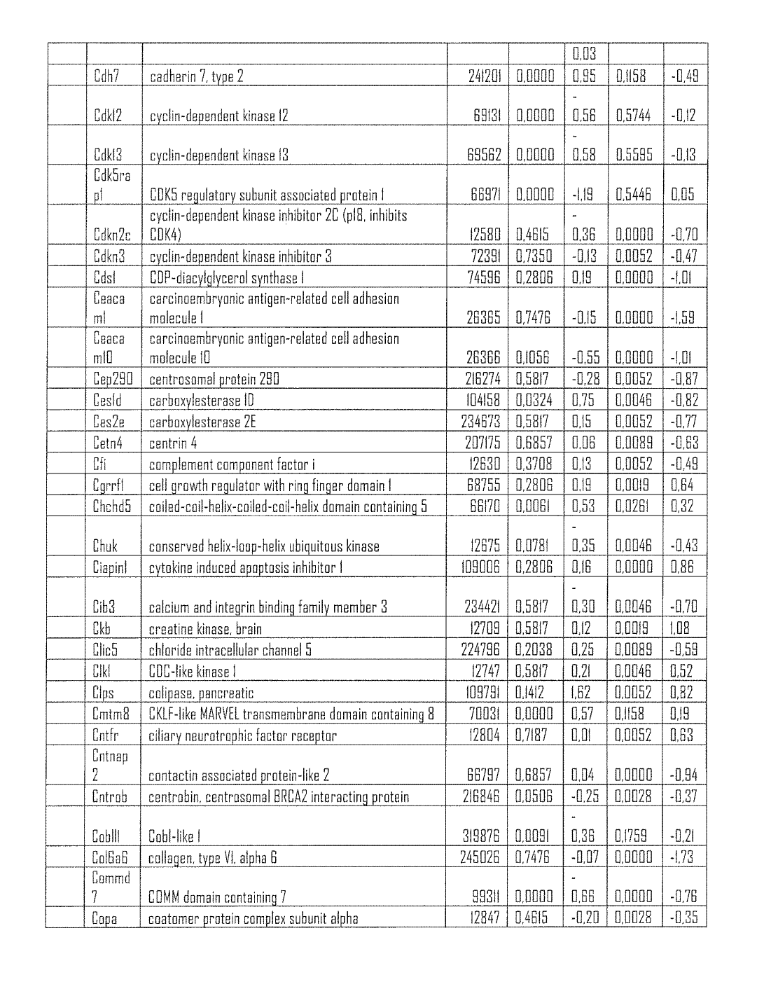

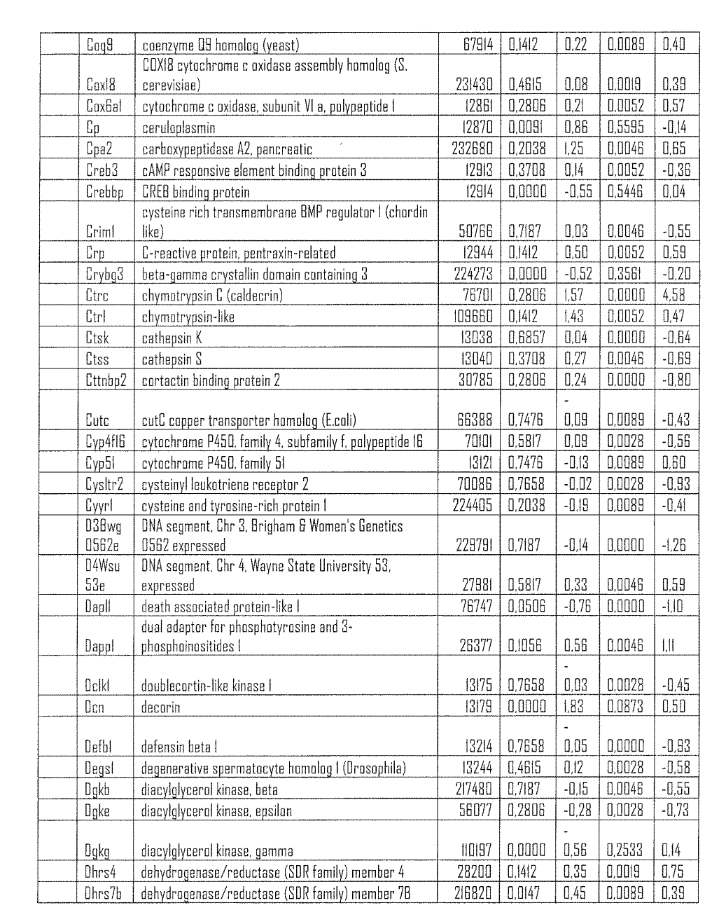

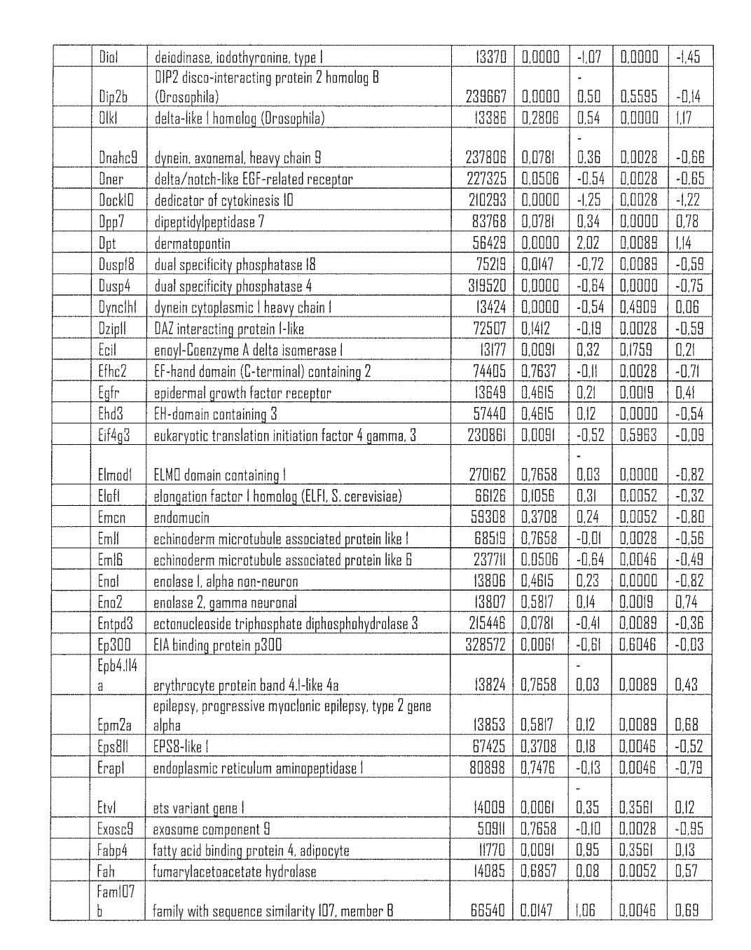

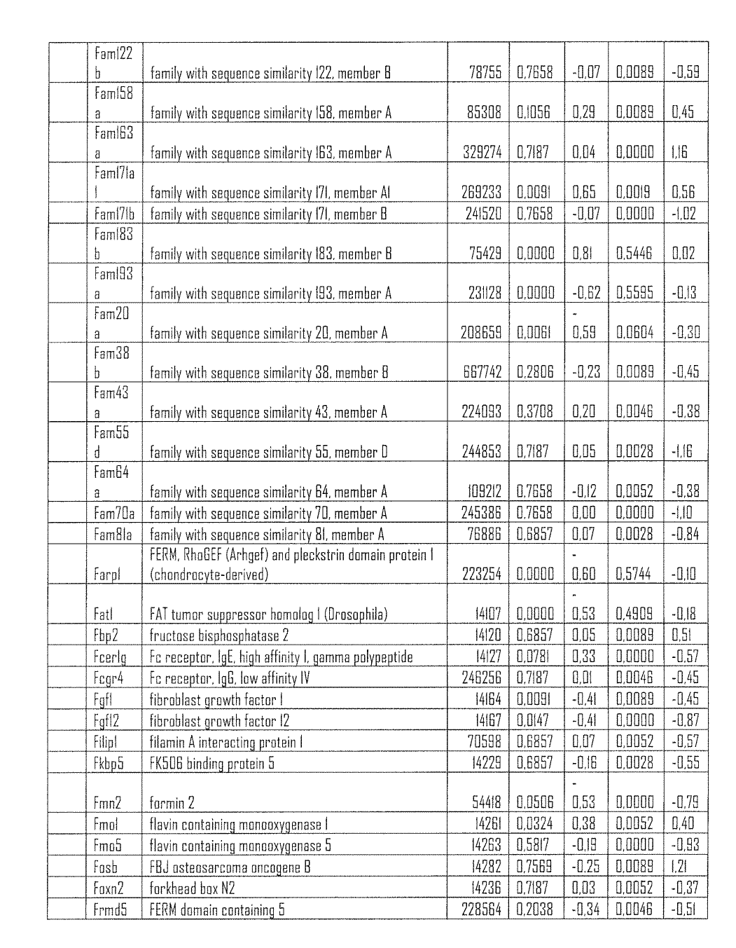

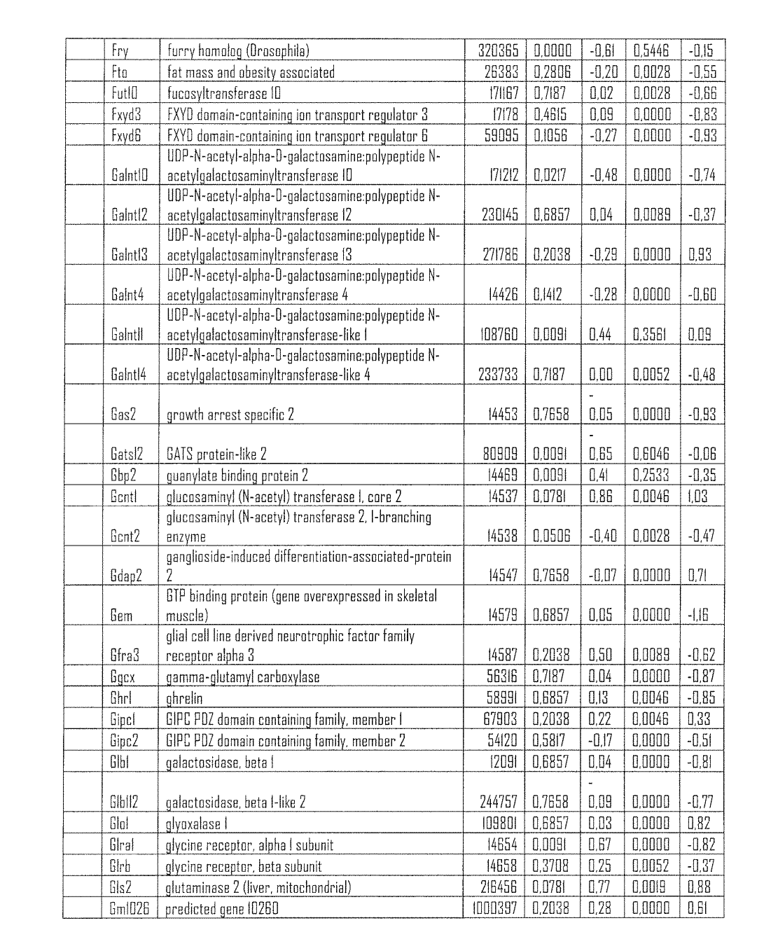

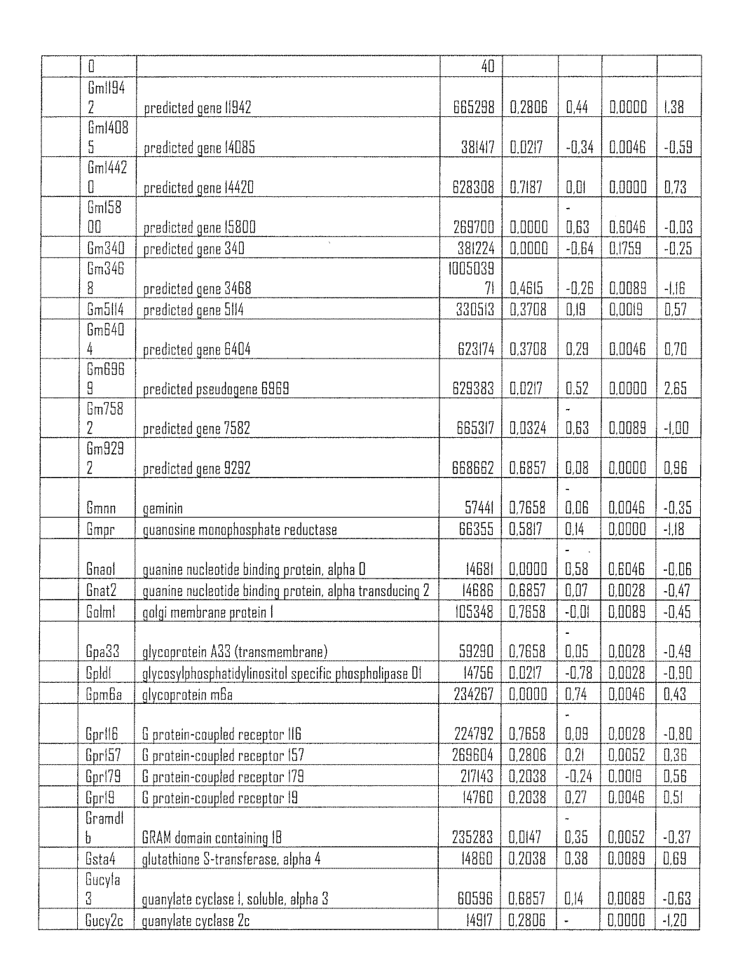

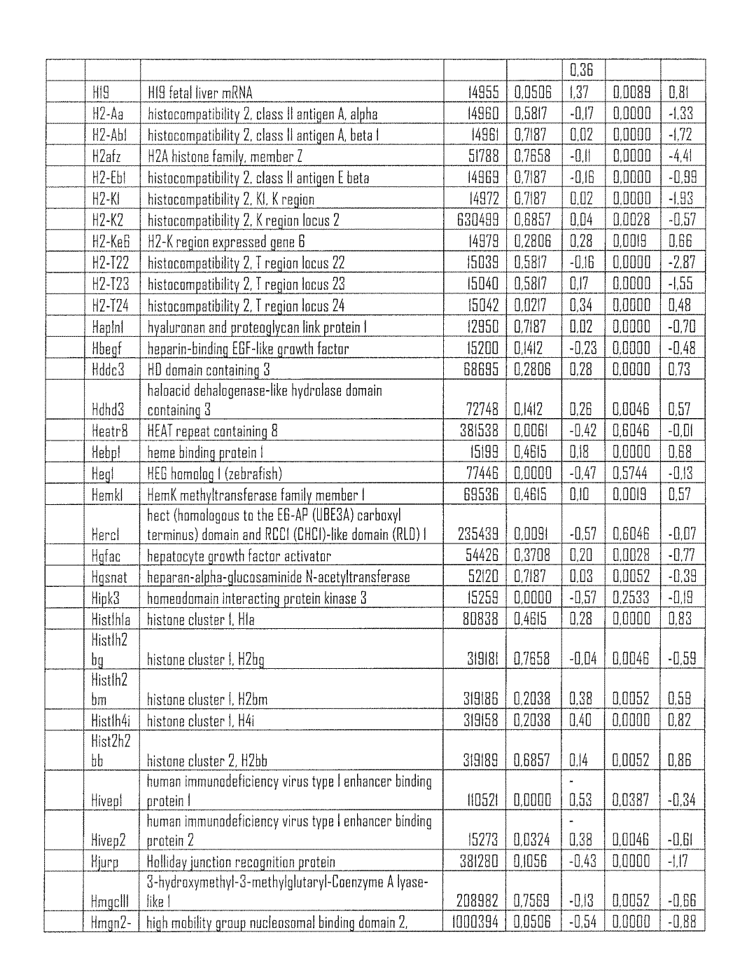

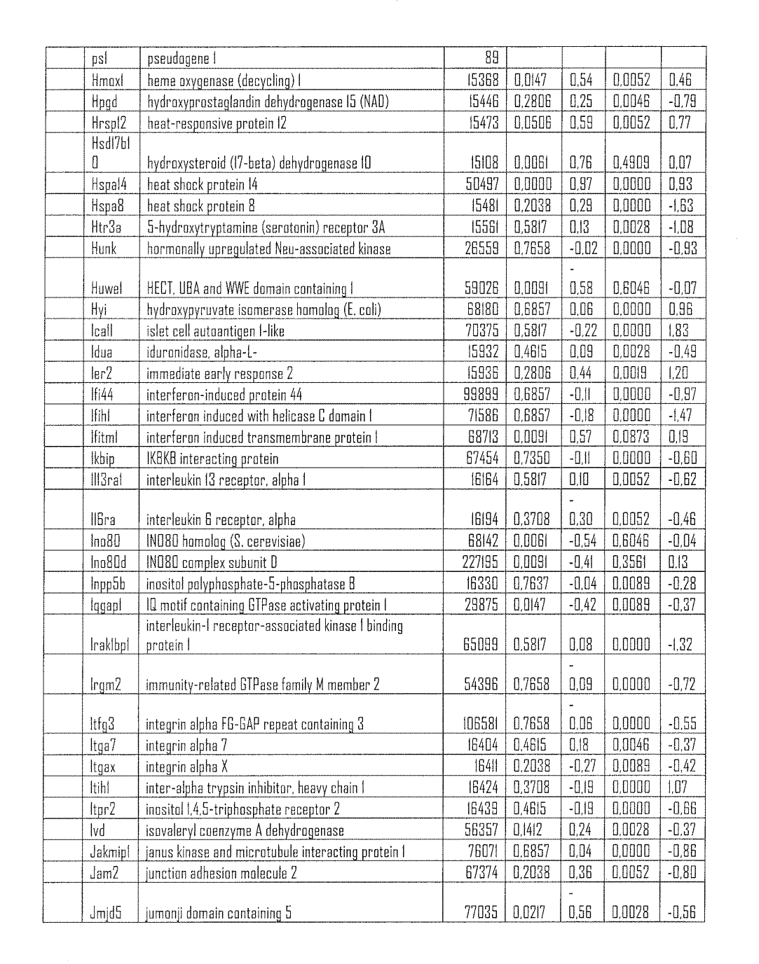

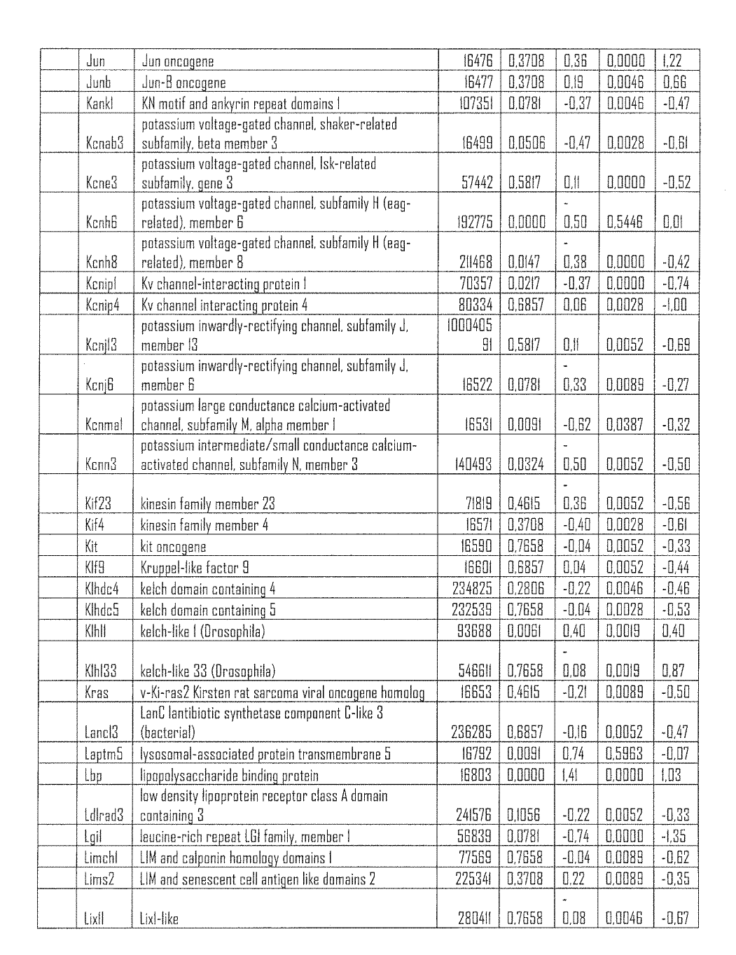

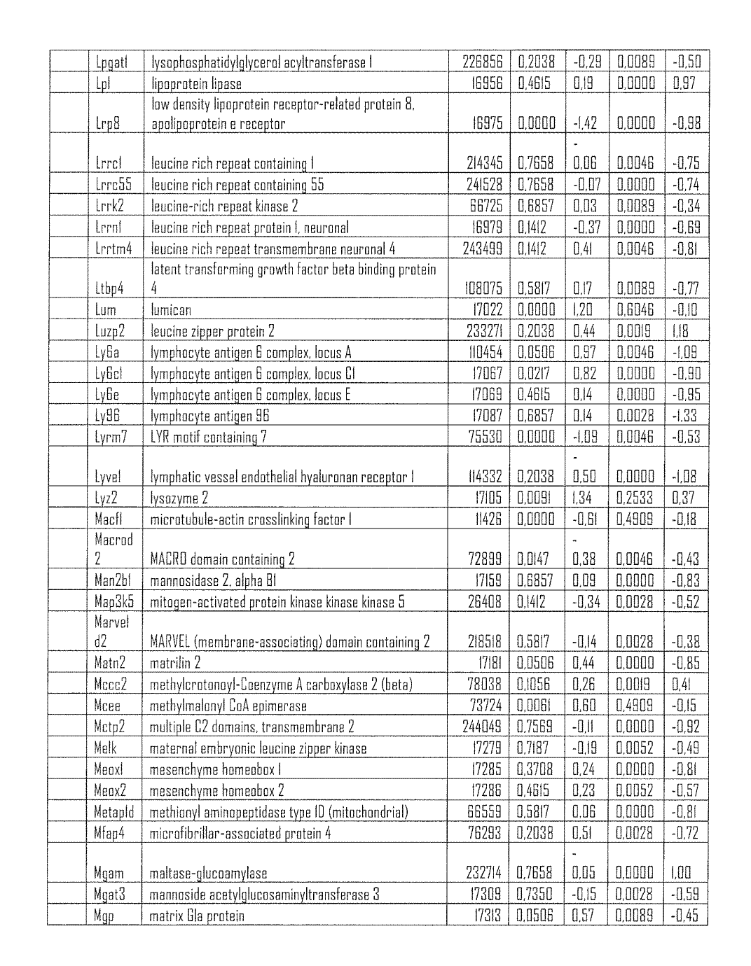

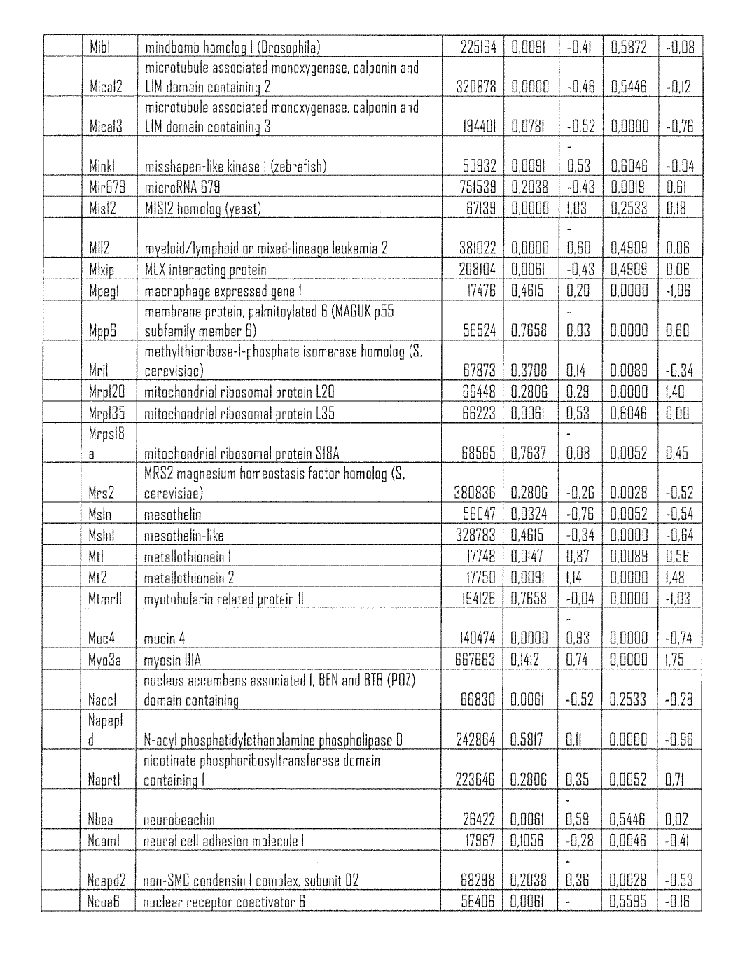

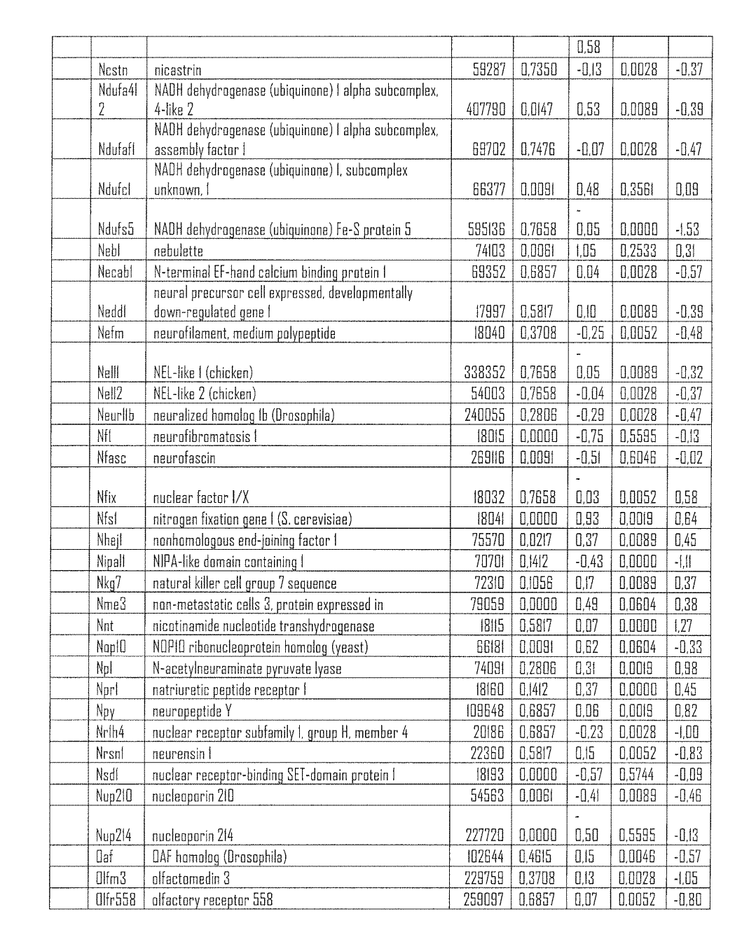

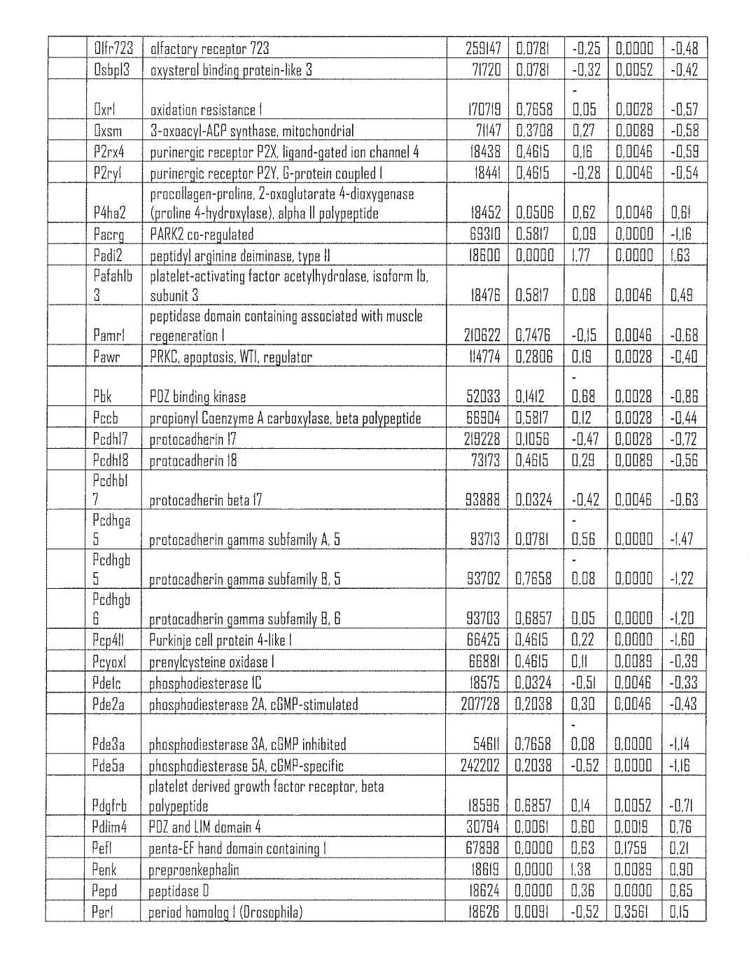

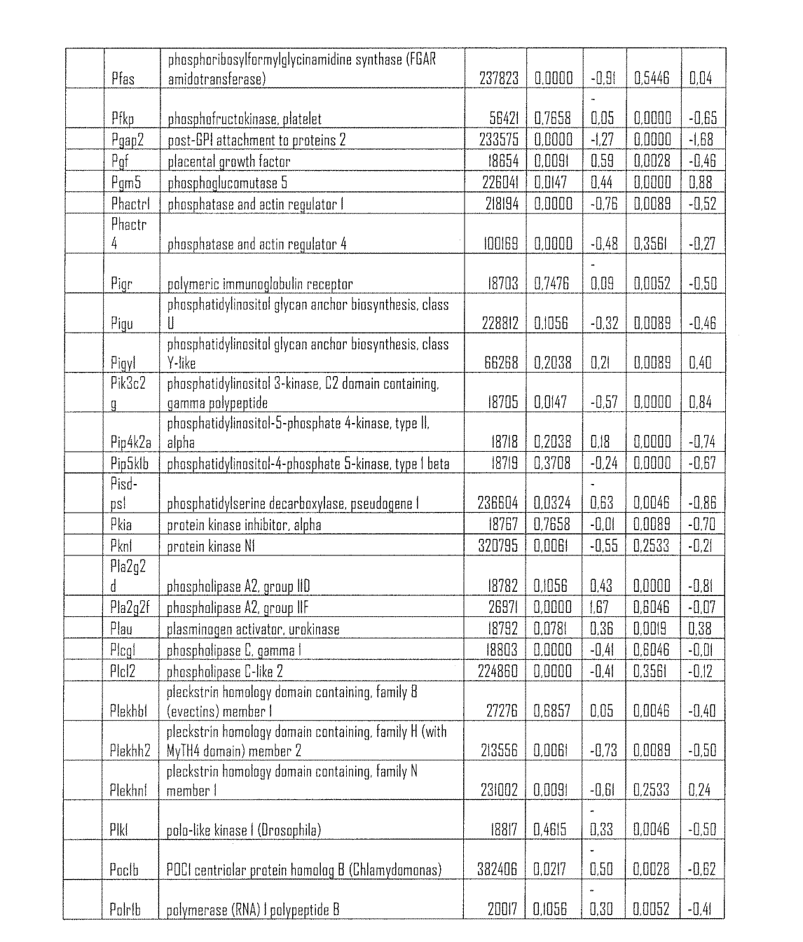

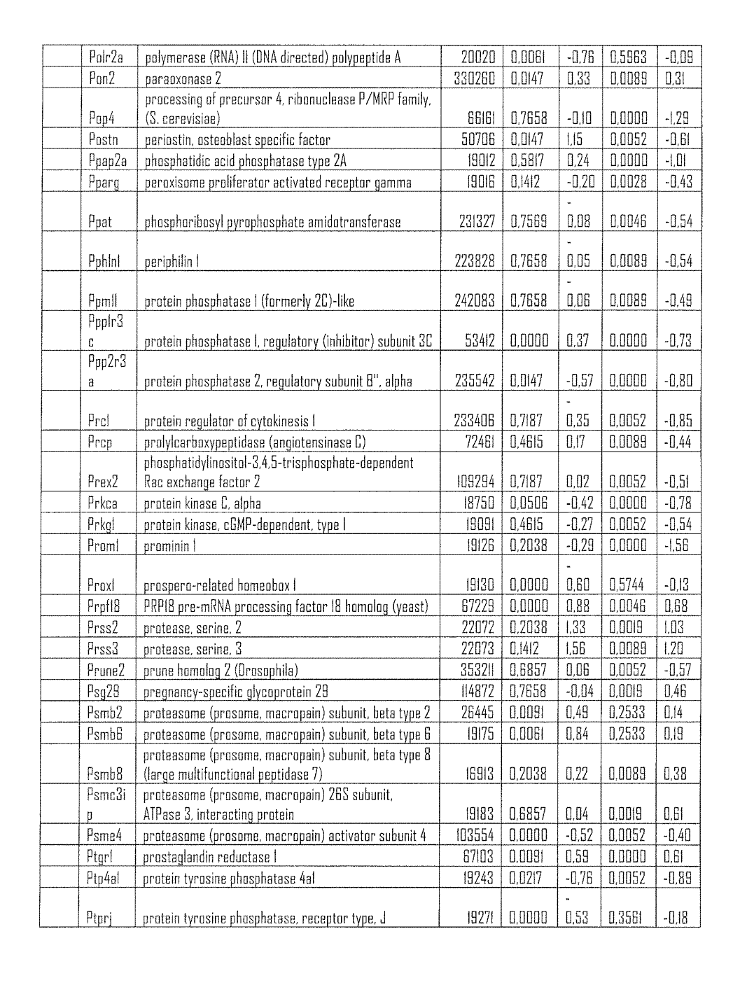

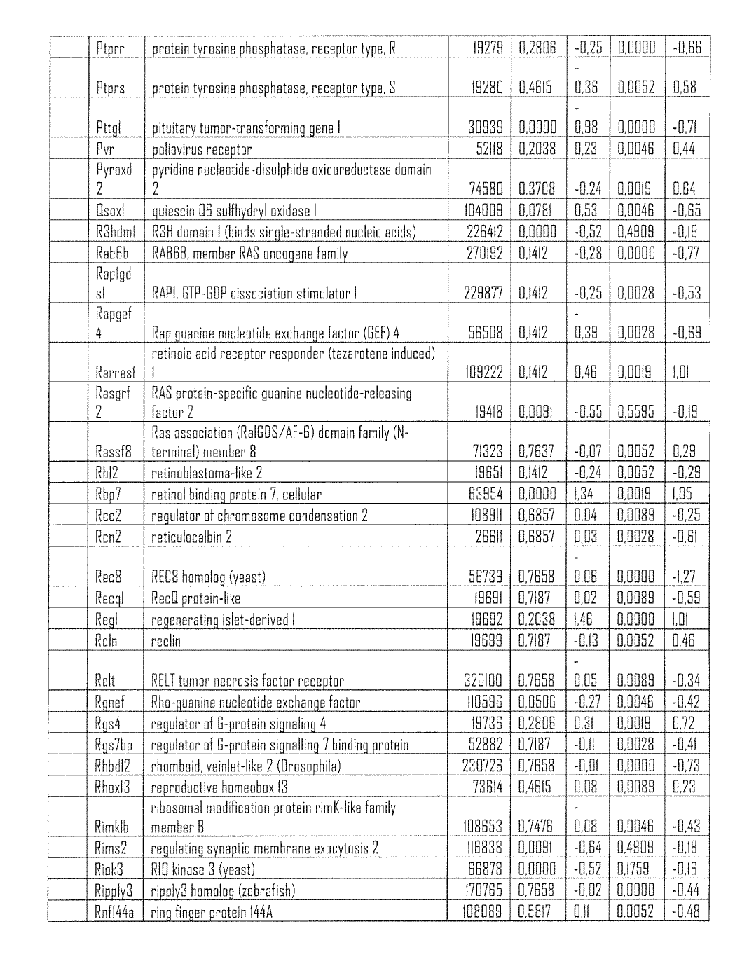

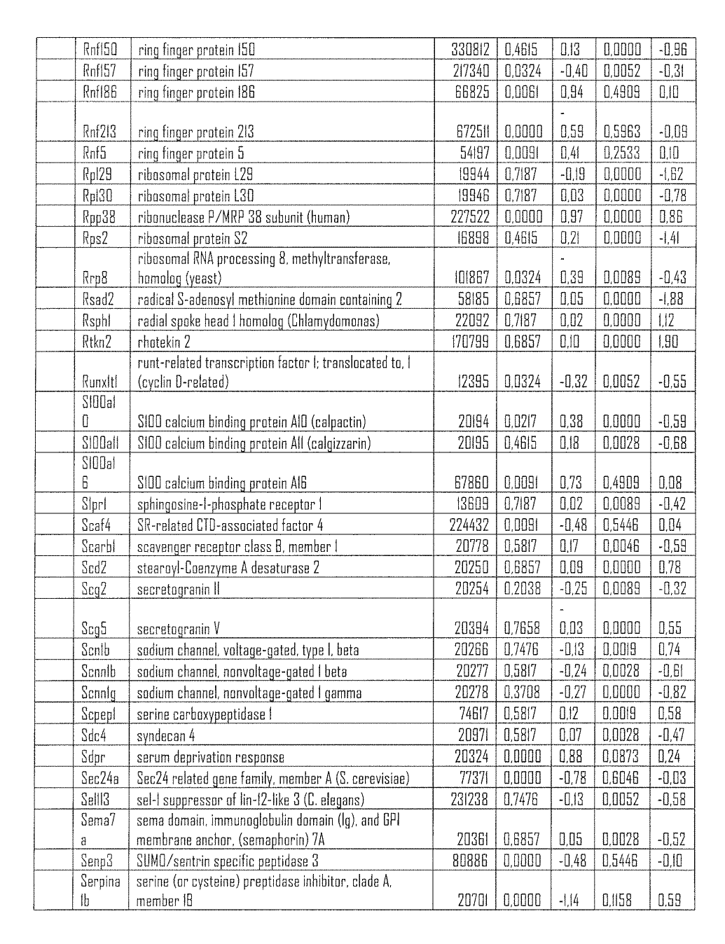

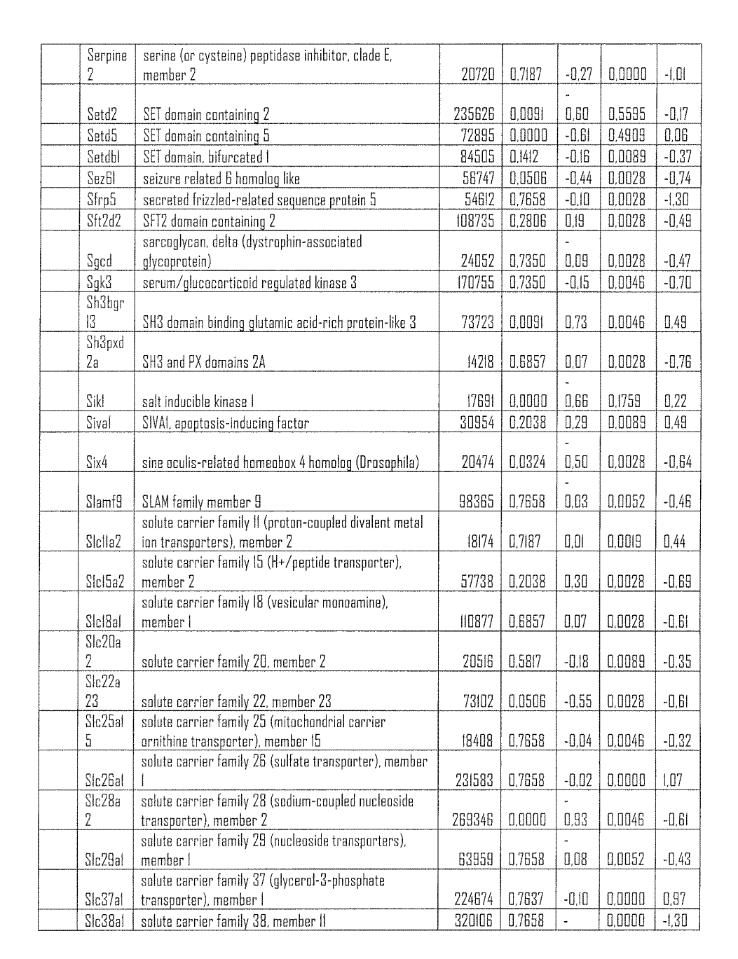

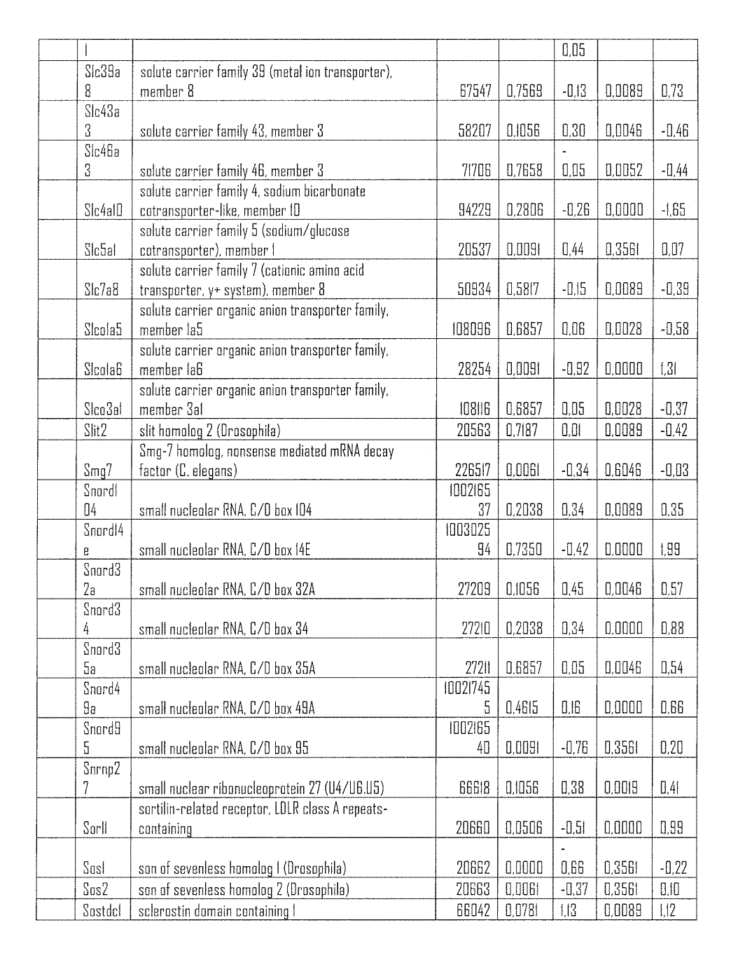

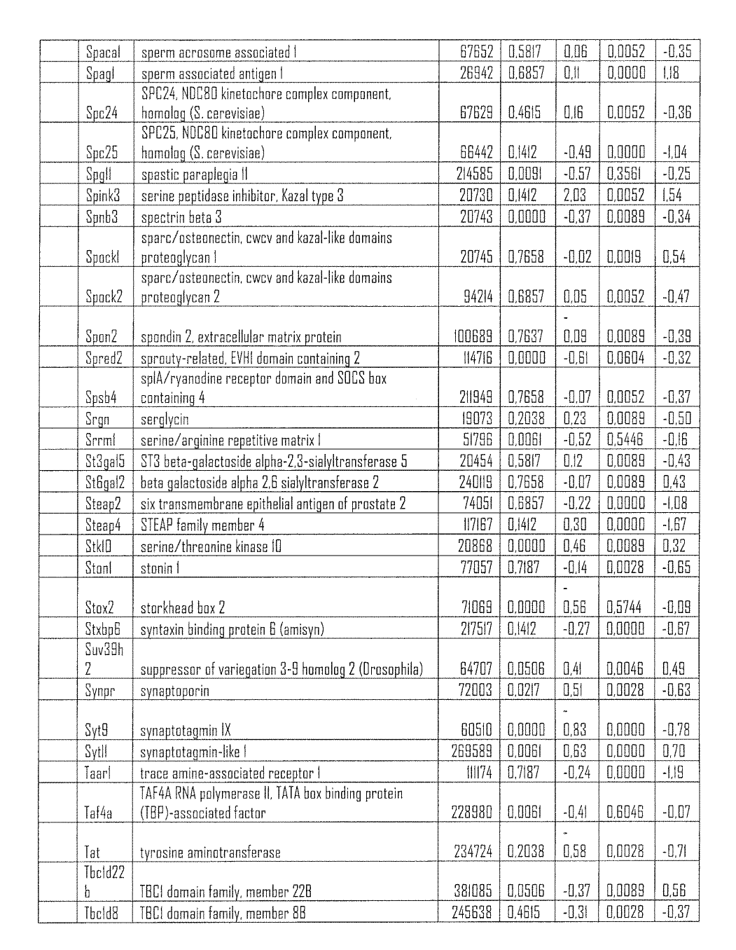

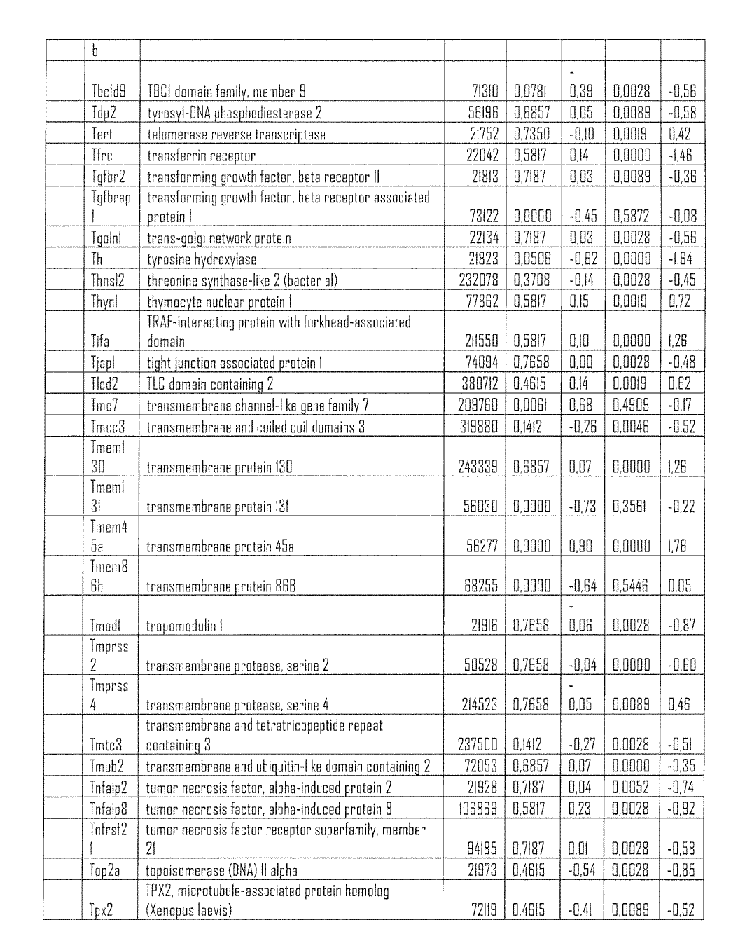

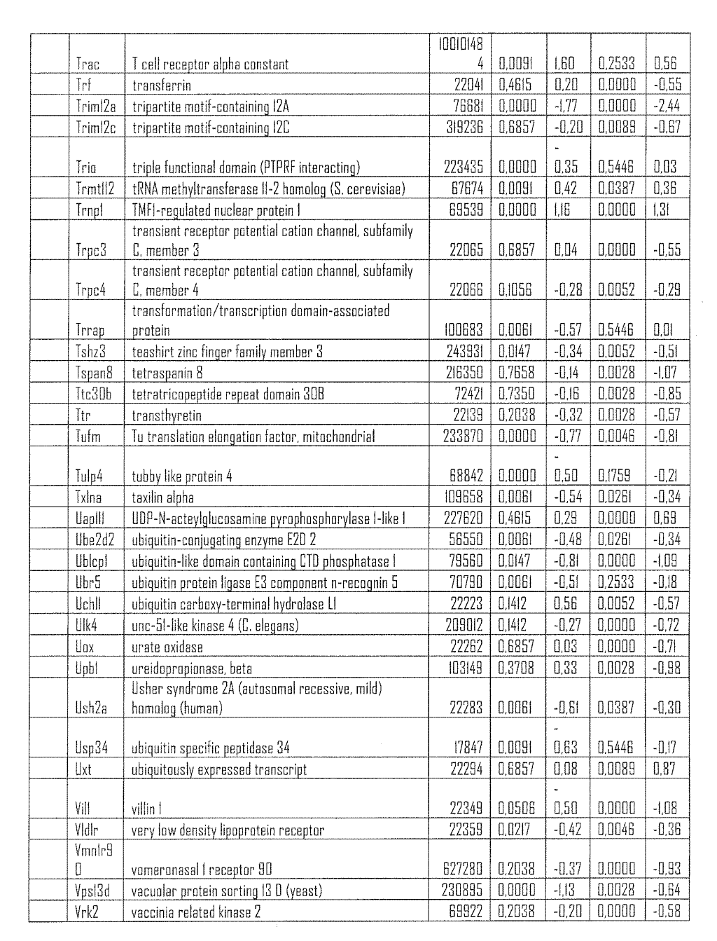

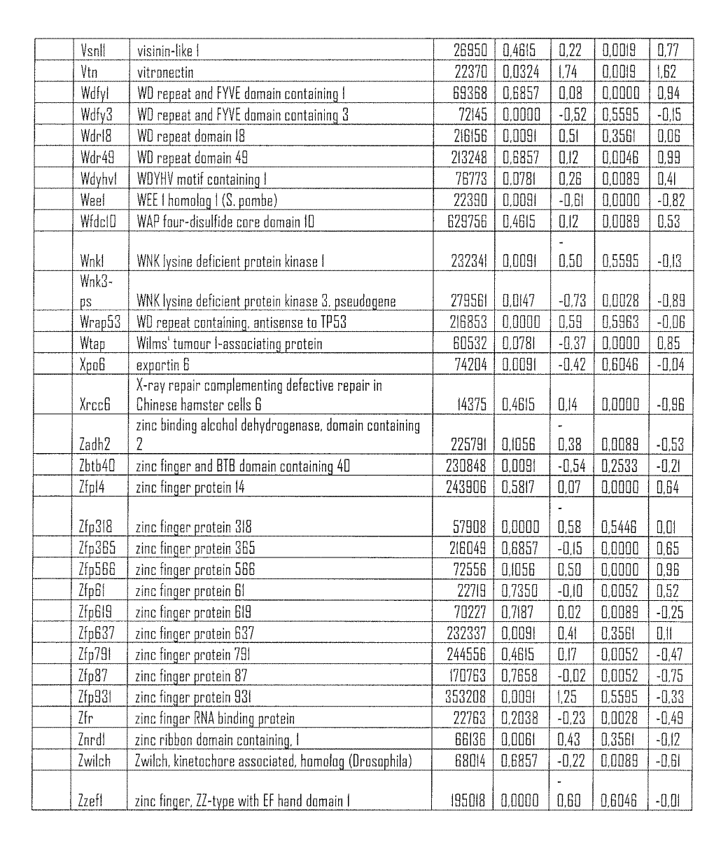

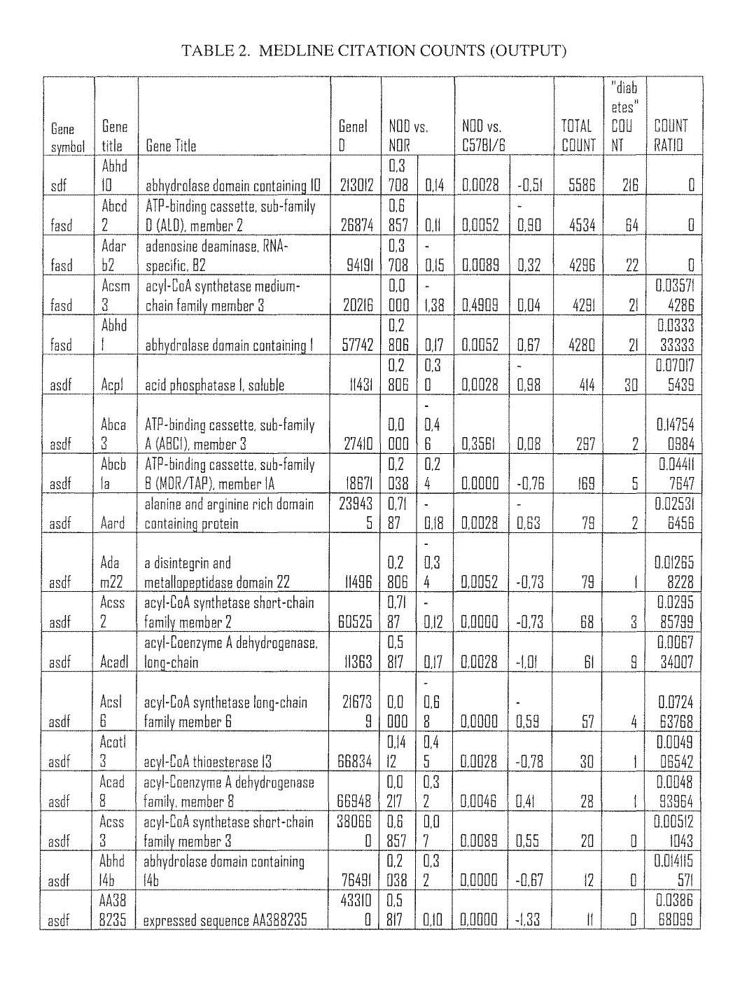

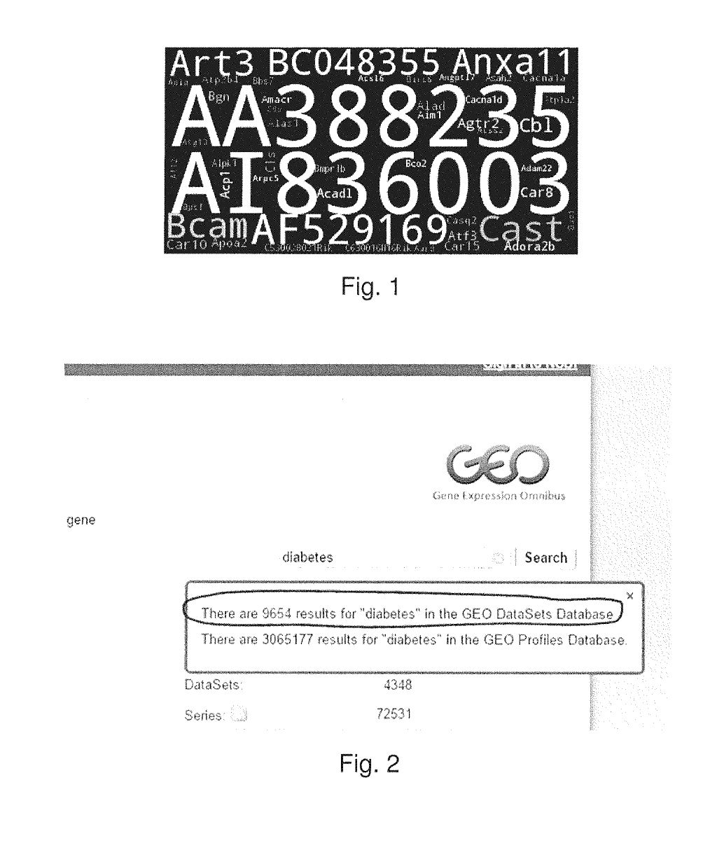

One output option is a “word cloud, as shown in FIG. 1, which converts the tabular data to a compact graphical form.

This technology provides the ability to cross reference gene name and symbol against public registry. Further, it provides the ability to cross reference gene name symbol and keywords against public registry, and report ratios of the above. It is noted that, since the automation serves to populate a spreadsheet file, any arbitrary mathematical or logical functions may be programmed into the spreadsheet, independent of the populating program.

The technology further has an ability to prioritize the results, and report as a word cloud, for example. As preferably implemented, the technology seeks to prioritize data based on a balance between availability of sufficient information about a gene or genetic sequence, and the sparsity or rarity of the published literature relating to a search topic. This, in turn, permits a researcher to select, for further investigation, genes for which a body of literature is available, but which has not been fully investigated according to the topic of interest.

### Example 1

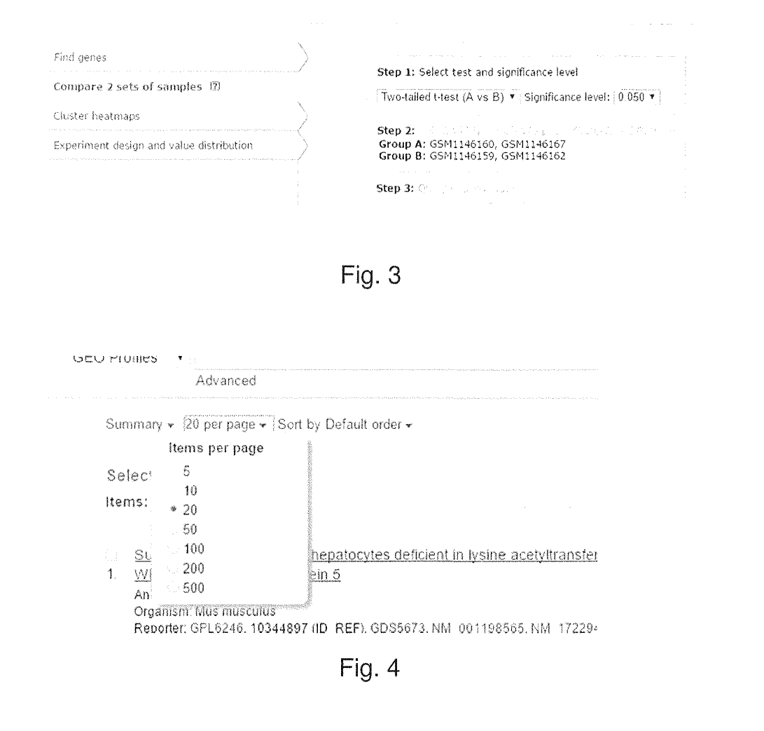

This example describes an operative example using the preferred embodiment of the technology, a program written in Python 3. Initially, a keyword is entered to search the NCBI GEO database (www.ncbi.nlm.nih.gov/geo). The user or automated agent then clicks on the datasets results, and a result of interest. See FIG. 2. The option, Compare 2 sets of samples is selected, and sample groups selected to analyze gene fluctuations. The link is followed, leading to the profile data results. See FIG. 3. To facilitate analysis, the Items per page is changed to 500. See FIG. 4. The Download profile data button is then selected (in the right margin), and the.txt document (ASCII) is converted to an .xlsx document (Microsoft Excel®).

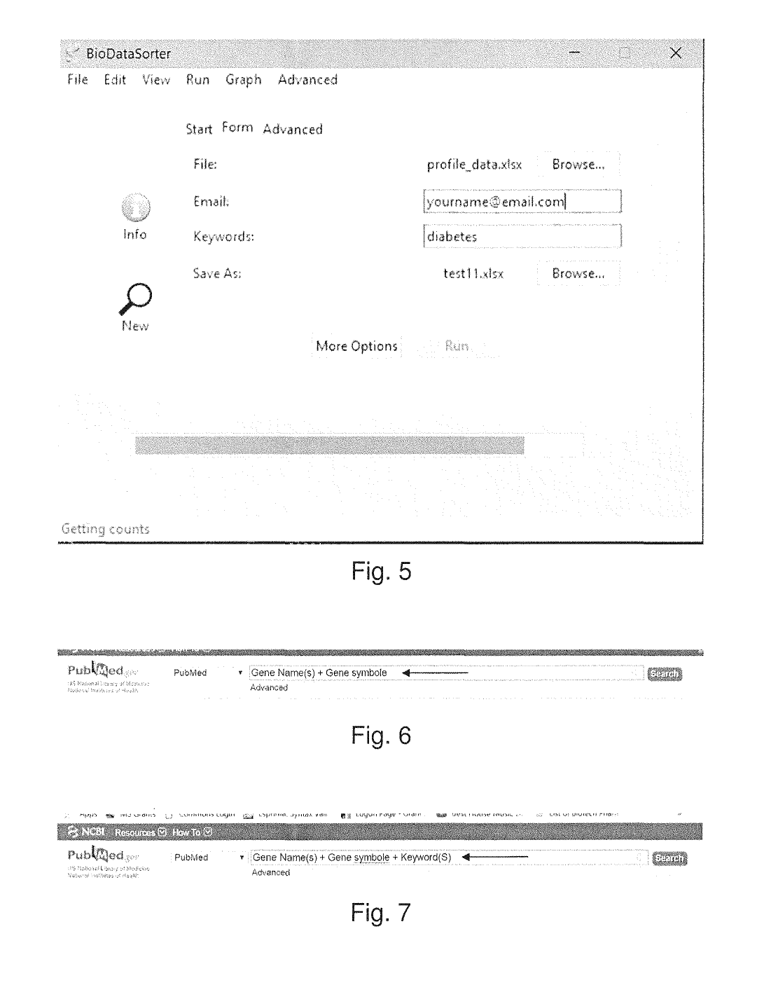

In BioDataSorter, the GEO file created as above is provided as an input file. See FIG. 5. “More Options” (right click) is selected, and the Symbol Column is changed to the input's “Symbol” or “Gene Symbol” column letter. The Synonyms Column is changed to the input's “Synonyms” or “Gene Title” column letter. Other options may also be selected, to include in the output. The program is then run, from the Form page or from the Run Menu. The process may take, e.g., up to 20 minutes to execute, depending on the number of genes being processed. The “Word Cloud” option in the “Graph” menu may be used to create a word cloud based on the output, as shown in FIG. 1.

Although embodiments of automated microarray data mining technology have been described in language specific to features and/or methods, the appended claims are not necessarily limited to the specific features or methods described. Rather, the specific features and methods are disclosed as example implementations.

TABLE 1
NCBI GEO DATA (INPUT)

| | | | | | | NOD vs. | |
|---|---|---|---|---|---|---|---|
| | | | | NOD vs. NOR | | C5781/6 | |
| | | | | | Log2 | | Log2 |
| | Gene | | | | Fold | | Fold |
| Test | Symbol | Gene Title | GeneID | q-value | regulation | q-value | regulation |
| asdf | AA388235 | expressed sequence AA388235 | 433100 | 0.5817 | 0.10 | 0.0000 | −1.33 |
| asdf | Aard | alanine and arginine rich domain containing protein | 239435 | 0.7187 | −0.18 | 0.0028 | −0.63 |
| asdf | Abca3 | ATP-binding cassette, sub-family A (ABCI), member 3 | 27410 | 0.0000 | −0.46 | 0.3561 | 0.08 |
| asdf | Abcbla | ATP-binding cassette, sub-family B (MOR/TAP). | 18671 | 0.2038 | 0.24 | 0.0000 | −0.76 |
| | | member IA | | | | | |
| fasd | Abcd2 | ATP-binding cassette, sub-family D (ALD), member 2 | 26874 | 0.6857 | 0.11 | 0.0052 | −0.90 |
| fasd | Abhd1 | abhydrolase domain containing 1 | 57742 | 0.2806 | 0.17 | 0.0052 | 0.67 |
| sdf | Abhd10 | abhydrolase domain containing 10 | 213012 | 0.3708 | 0.14 | 0.0028 | −0.51 |
| asdf | Abhd14b | abhydrolase domain containing 14b | 76491 | 0.2038 | 0.32 | 0.0000 | −0.67 |
| asdf | Acad8 | acyl-Coenzyme A dehydrogenase family, member 8 | 66948 | 0.0217 | 0.32 | 0.0046 | 0.41 |
| asdf | Acadl | acyl-Coenzyme A dehydrogenase, long-chain | 11363 | 0.5817 | 0.17 | 0.0028 | −1.01 |
| asdf | Acat13 | acyl-CoA thioesterase 13 | 66834 | 0.1412 | 0.45 | 0.0028 | −0.78 |
| asdf | Asp1 | acid phosphatase 1, soluble | 11431 | 0.2806 | 0.30 | 0.0028 | −0.98 |
| asdf | Acs16 | acyl-CoA synthetase long-chain family member 6 | 216739 | 0.0000 | −0.68 | 0.0000 | −0.59 |
| fasd | Acsm3 | acyl-CoA synthetase medium-chain family member 3 | 20216 | 0.0000 | −1.38 | 0.4909 | 0.04 |
| asdf | Acss2 | acyl-CoA synthetase short-chain family member 2 | 60525 | 0.7187 | −0.12 | 0.0000 | −0.73 |
| asdf | Acss3 | acyl-CoA synthetase short-chain family member 3 | 380660 | 0.6857 | 0.07 | 0.0089 | 0.55 |
| asdf | Adam22 | a disintegrin and metallopeptidase domain 22 | 11496 | 0.2806 | −0.34 | 0.0052 | −0.73 |
| fasd | Adarb2 | adenosine deaminase, RNA-specific B2 | 94191 | 0.3708 | −0.15 | 0.0089 | 0.32 |
| | Adi1 | acireductone dioxygenase 1 | 104923 | 0.4615 | 0.12 | 0.0000 | 0.87 |
| | Adora2b | adenosine A2b receptor | 11541 | 0.0061 | 0.96 | 0.1759 | 0.39 |
| | AF529169 | cDNA sequence AF529169 | 209743 | 0.0061 | −0.51 | 0.0873 | 0.30 |
| | Afap1l2 | actin filament associated protein 1-like 2 | 226250 | 0.3708 | 0.17 | 0.0052 | −0.39 |
| | Aff2 | AF4/FMR2 family, member 2 | 14266 | 0.0061 | −0.60 | 0.0028 | −0.61 |
| | Agtr2 | angiotensin II receptor, type 2 | 11609 | 0.2806 | 0.36 | 0.0089 | 0.46 |
| | A1836003 | expressed sequence A1836003 | 239650 | 0.0506 | −0.36 | 0.0000 | −0.93 |
| | Aim1 | absent in melanoma 1 | 11630 | 0.1412 | −0.40 | 0.0028 | −0.72 |
| | Akap13 | A kinase (PRKA) anchor protein 13 | 75547 | 0.0147 | −0.60 | 0.0000 | 0.55 |
| | Akap6 | A kinase (PRKA) anchor protein 6 | 238161 | 0.5817 | −0.21 | 0.0000 | −1.02 |
| | Akirin2 | akirin 2 | 433693 | 0.2038 | 0.19 | 0.0000 | 0.50 |
| | Akr1c14 | aldo-keto reductase family 1, member C14 | 105387 | 0.7658 | −0.12 | 0.0028 | −0.99 |
| | Akr1e1 | aldo-keto reductase family 1, member E1 | 56043 | 0.0000 | −1.04 | 0.0000 | −1.49 |
| | Alad | aminolevulinate, delta-, dehydratase | 17025 | 0.4615 | 0.14 | 0.0000 | 0.83 |
| | Alas1 | aminolevulinic acid synthase 1 | 11655 | 0.6857 | 0.08 | 0.0028 | −0.50 |
| | Alg1 | asparagine-linked glycosylation 1 homolog (yeast, beta- | 208211 | 0.6857 | 0.03 | 0.0028 | −0.44 |
| | | 1,4-mannosyltransferase) | | | | | |
| | Alg9 | asparagine-linked glycosylation 9 homolog (yeast, | 102580 | 0.2038 | −0.22 | 0.0028 | −0.41 |
| | | alpha 1,2 mannosyltransferase) | | | | | |
| | Alpk1 | alpha-kinase 1 | 71481 | 0.1412 | −0.44 | 0.0000 | 1.48 |
| | Amacr | alpha-methylacyl-CoA racemase | 17117 | 0.1412 | 0.28 | 0.0019 | 0.50 |
| | Angpt17 | angiopoietin-like 7 | 654812 | 0.0000 | 2.26 | 0.0000 | 2.31 |
| | Ankrd54 | ankyrin repeat domain 54 | 223690 | 0.2806 | 0.23 | 0.0000 | 0.50 |
| | Anubll | ANI, ubiquitin-like, homolog (Xenopus laevis) | 67492 | 0.3708 | −0.24 | 0.0046 | −0.37 |
| | Anxall | annexin All | 11744 | 0.4615 | 0.22 | 0.0000 | −0.56 |
| | Ap1s1 | adaptor protein complex AP-1, sigma 1 | 11769 | 0.2806 | 0.22 | 0.0089 | −0.28 |
| | Apip | APAFI interacting protein | 56369 | 0.0091 | 0.51 | 0.1158 | 0.31 |
| | Apoa2 | apolipoprotein A-II | 11807 | 0.6857 | 0.02 | 0.0000 | −1.58 |
| | Arfgef2 | ADP-ribosylation factor guanine nucleotide-exchange | 99371 | 0.0091 | −0.49 | 0.6046 | −0.04 |
| | | factor 2 (brefeldin A-inhibited) | | | | | |
| | Arhgap18 | Rho GTPase activating protein 18 | 73910 | 0.0000 | 1.91 | 0.5595 | −0.20 |
| | Arhgap21 | Rho GTPase activating protein 21 | 71435 | 0.0000 | −0.52 | 0.6046 | −0.03 |
| | Arhgap32 | Rho GTPase activating protein 32 | 330914 | 0.0000 | −0.66 | 0.5446 | −0.22 |
| | Arhgap36 | Rho GTPase activating protein 36 | 75404 | 0.1412 | 0.76 | 0.0000 | 2.18 |
| | Arhgef15 | Rho guanine nucleotide exchange factor (GEF) 15 | 442801 | 0.0217 | −0.38 | 0.0052 | −0.36 |
| | Arhgef16 | Rho guanine nucleotide exchange factor (GEF) 16 | 230972 | 0.7658 | −0.01 | 0.0028 | −0.55 |
| | Arid1a | AT rich interactive domain 1A (SWI-like) | 93760 | 0.0091 | −0.65 | 0.6049 | −0.08 |
| | Arl4d | ADP-ribosylation factor-like 40 | 80981 | 0.1412 | −0.25 | 0.0052 | 0.40 |
| | Arpc5 | actin related protein 2/3 complex, subunit 5 | 67771 | 0.5817 | 0.15 | 0.0028 | −0.72 |
| | Art3 | ADP-ribosyltransferase 3 | 109979 | 0.2806 | 0.41 | 0.0028 | −1.13 |
| | Asah2 | N-acylsphingasine amidohydrolase 2 | 54447 | 0.7350 | −0.14 | 0.0000 | −1.29 |
| | Asf1b | ASF1 anti-silencing function 1 homolog B (S. cerevisiae) | 66929 | 0.7658 | −0.13 | 0.0052 | −0.52 |
| | Ashl1 | ashl (absent, small, or homeotic)-like (Drosophila) | 192195 | 0.0091 | −0.57 | 0.6046 | −0.02 |
| | Atf3 | activating transcription factor 3 | 11910 | 0.7187 | 0.05 | 0.0019 | 0.77 |
| | Atg13 | ATG13 autophagy related 13 homolog (S. cerevisiae) | 51897 | 0.7187 | −0.17 | 0.0089 | −0.39 |
| | Atox1 | ATX1 (antioxidant protein 1) homolog 1 (yeast) | 11927 | 0.0000 | 0.90 | 0.6046 | −0.05 |
| | Atp10d | ATPase, class V, type 100 | 231287 | 0.7658 | −0.06 | 0.0000 | 0.56 |
| | Atp13a3 | ATPase type 13A3 | 224088 | 0.0147 | −0.38 | 0.0000 | −0.39 |
| | Atpla2 | ATPase, Na+/K+ transporting, alpha 2 polypeptide | 98660 | 0.7658 | −0.03 | 0.0028 | −0.49 |
| | Atp2b4 | ATPase, Ca++ transporting, plasma membrane 4 | 381290 | 0.0324 | −0.44 | 0.0028 | −0.84 |
| | Atp6vOe2 | ATPase, H+ transporting, lysosomal VO subunit E2 | 76252 | 0.2038 | 0.25 | 0.0000 | −0.57 |
| | Aurkaip1 | aurora kinase A interacting protein 1 | 66077 | 0.0506 | 0.38 | 0.0089 | −0.41 |
| | B3galt5 | UDP-Gal:betaGlcN4c beta 1,3-galactosyltransferase, | 93961 | 0.2038 | −0.39 | 0.0052 | −0.75 |
| | | polypeptide 5 | | | | | |
| | Baz2a | bromodomain adjacent to zinc finger domain, 2A | 116848 | 0.0061 | −0.65 | 0.5963 | −0.09 |
| | Bbs7 | Bardet-Biedl syndrome 7 (human) | 71492 | 0.7350 | −0.18 | 0.0089 | −0.81 |
| | BCD48355 | cDNA sequence BCD48355 | 381101 | 0.5817 | 0.08 | 0.0046 | −0.50 |
| | BCD56474 | cDNA sequence BCD56474 | 414077 | 0.5817 | 0.08 | 0.0019 | 0.89 |
| | Bcam | basal cell adhesion molecule | 57278 | 0.4615 | 0.18 | 0.0089 | −0.60 |
| | Bcl6b | B cell CLL/lymphoma 6, member B | 12029 | 0.0091 | 0.63 | 0.0134 | −0.50 |
| | Bco2 | beta-carotene oxygenase 2 | 170752 | 0.6857 | 0.05 | 0.0000 | −0.89 |
| | Bgn | biglycan | 12111 | 0.2038 | 0.77 | 0.0046 | −0.55 |
| | Birc6 | baculoviral IAP repeat-containing 6 | 12211 | 0.0000 | −0.69 | 0.6046 | −0.01 |
| | Bmpr1b | bone morphogenetic protein receptor, type 1B | 12167 | 0.6857 | 0.07 | 0.0000 | 1.07 |
| | Bpnt1 | bisphosphate 3′-nucleotidase 1 | 23827 | 0.2038 | 0.33 | 0.0089 | −0.57 |
| | Bptf | bromodomain PHD finger transcription factor | 207165 | 0.0061 | −0.54 | 0.6046 | −0.03 |
| | Btnl9 | butyrophilin-like 9 | 237754 | 0.2038 | 0.30 | 0.0028 | −0.54 |
| | Bub1 | budding uninhibited by benzimidazoles 1 | 12235 | 0.7187 | −0.44 | 0.0089 | −0.82 |
| | | homolog (S. cerevisiae) | | | | | |
| | Bub1b | budding uninhibited by benzimidazoles 1 homolog, beta | 12236 | 0.7658 | −0.09 | 0.0000 | −0.59 |
| | | (S. cerevisiae) | | | | | |
| | C1s | complement component 1, s subcomponent | 50908 | 0.0324 | 0.89 | 0.0046 | −1.05 |
| | C2 | complement component 2 (within H-2S) | 12263 | 0.2038 | 0.26 | 0.0000 | 0.71 |
| | C2cd4b | C2 calcium-dependent domain containing 4B | 75697 | 0.7569 | −0.10 | 0.0089 | 0.66 |
| | C530028021Rik | RIKEN cDNA C530028021 gene | 319352 | 0.7187 | −0.17 | 0.0000 | 1.72 |
| | C630016N16Rik | RIKEN cDNA C630016N16 gene | 791088 | 0.4615 | −0.26 | 0.0089 | −0.65 |
| | C8b | complement component 8, beta polypeptide | 110382 | 0.4615 | 0.30 | 0.0000 | −2.04 |
| | Cacna1a | calcium channel, voltage-dependent, P/Q type, alpha 1A | 12286 | 0.0091 | −0.75 | 0.0873 | 0.35 |
| | | subunit | | | | | |
| | Cacna1d | calcium channel, voltage-dependent, L type, alpha 1D | 12289 | 0.0061 | −0.75 | 0.6046 | −0.02 |
| | | subunit | | | | | |
| | Cap2 | CAP, adenylate cyclase-associated protein, 2 (yeast) | 67252 | 0.6857 | −0.13 | 0.0028 | −0.57 |
| | Capg | capping protain (actin filament), gelsolin-like | 12332 | 0.1412 | 0.26 | 0.0046 | −0.68 |
| | Car10 | carbonic anhydrase 10 | 72605 | 0.4615 | −0.29 | 0.0000 | −0.86 |
| | Car15 | carbonic anhydrase 15 | 80733 | 0.0000 | 0.68 | 0.0000 | 0.98 |
| | Car8 | carbonic anhydrase 8 | 12319 | 0.2806 | 0.15 | 0.0052 | −0.34 |
| | Casq2 | calsequestrin 2 | 12373 | 0.7476 | −0.10 | 0.0028 | −0.67 |
| | Cast | calpastatin | 12380 | 0.7658 | −0.04 | 0.0052 | −0.42 |
| | Cbl | Casitas B-lineag lymphoma | 12402 | 0.0000 | −0.41 | 0.6046 | −0.04 |
| | Cbln2 | cerebellin 2 precursor protein | 12405 | 0.0061 | 0.71 | 0.0019 | 0.66 |
| | Cbln4 | cerebellin 4 precursor protein | 228942 | 0.0506 | 0.52 | 0.0000 | 0.72 |
| | Cbs | cystathionine beta-synthase | 12411 | 0.5817 | 0.29 | 0.0089 | 1.04 |
| | Chx7 | chromobox homolog 7 | 52609 | 0.7658 | −0.01 | 0.0000 | −0.49 |
| | Ccdc103 | coiled-coil domain containing 103 | 73293 | 0.2806 | 0.23 | 0.0089 | −0.45 |
| | Ccdc68 | coiled-coil domain containing 68 | 381175 | 0.5817 | 0.16 | 0.0000 | 1.10 |
| | Ccdc72 | coiled-coil domain containing 72 | 66167 | 0.5817 | −0.34 | 0.0000 | −1.97 |
| | Ccdc80 | coiled-coil domain containing 80 | 67896 | 0.1412 | 0.76 | 0.0089 | −0.41 |
| | Ccna2 | cyclin A2 | 12428 | 0.3708 | −0.52 | 0.0089 | −0.53 |
| | Ccnd1 | cyclin D1 | 12443 | 0.0506 | −0.28 | 0.0000 | −0.82 |
| | Cd164l2 | Cd164 sialomucin-like 2 | 59655 | 0.0147 | 0.71 | 0.0019 | 0.66 |
| | Cd300lg | CD300 antigen like family member G | 52685 | 0.4615 | 0.17 | 0.0028 | −0.46 |
| | Cd40 | CD40 antigen | 21939 | 0.5817 | 0.07 | 0.0019 | 0.49 |
| | Cd44 | CD44 antigen | 12505 | 0.0000 | −0.52 | 0.5446 | 0.01 |
| | Cd59a | CD59a antigen | 12509 | 0.0217 | −0.57 | 0.0048 | 0.86 |
| | Cd72 | CD72 antigen | 12517 | 0.4815 | 0.12 | 0.0000 | −0.81 |
| | Cd74 | CD74 antigen (invariant polypeptide of major | 16149 | 0.7187 | −0.20 | 0.0028 | −1.17 |
| | | histocompatibility complex, class II antigen-associated) | | | | | |
| | Cd93 | CD93 antigen | 17064 | 0.7658 | −0.09 | 0.0052 | −0.69 |
| | Cdc42bpb | CDC42 binding protein kinase beta | 217866 | 0.0091 | −0.48 | 0.5595 | −0.12 |
| | Cdca3 | cell division cycle associated 3 | 14793 | 7476 | −0.15 | 0.0046 | −0.46 |
| | Cdh19 | cadherin 19, type 2 | 227485 | 0.7658 | −0.03 | 0.0000 | −1.07 |
| | Cdh7 | cadherin 7, type 2 | 241201 | 0.0000 | 0.95 | 0.1158 | −0.49 |
| | Cdk12 | cyclin-dependent kinase 12 | 69131 | 0.0000 | −0.56 | 0.5744 | −0.12 |
| | Cdk13 | cyclin-dependent kinase 13 | 69562 | 0.0000 | −0.58 | 0.5585 | −0.13 |
| | Cdk5rap1 | CDK5 regulatory subunit associated protein 1 | 66971 | 0.0000 | −1.19 | 0.5446 | 0.05 |
| | Cdkn2c | cyclin-dependent kinase inhibitor 2C (p18 inhibits | 12580 | 0.4615 | −0.36 | 0.0000 | −0.70 |
| | | CDK4) | | | | | |
| | Cdkn3 | cyclin-dependent kinase inhibitor 3 | 72391 | 0.7350 | −0.13 | 0.0052 | −0.47 |
| | Cds1 | CDP-diacylglycerol synthase 1 | 74596 | 0.2806 | 0.19 | 0.0000 | −1.01 |
| | Ceacam1 | carcinoembryonic antigen-related cell adhesion 1 | 26365 | 0.7476 | −0.15 | 0.0000 | −1.59 |
| | | molecule 1 | | | | | |
| | Ceacam10 | carcinoembryonic antigen-related cell adhesion | 26366 | 0.1056 | −0.55 | 0.0000 | −0.01 |
| | | molecule 10 | | | | | |
| | Cep290 | centrosomal protein 290 | 216274 | 0.5817 | −0.28 | 0.0052 | −0.87 |
| | Ce1d | carboxylesterase 1D | 104158 | 0.0324 | 0.75 | 0.0046 | −0.82 |
| | Ces2e | carboxylesterase 2E | 234673 | 0.5817 | 0.15 | 0.0052 | −0.77 |
| | Cetn4 | centrin 4 | 207175 | 0.6857 | 0.06 | 0.0089 | −0.63 |
| | Cfi | complement component factor i | 12630 | 0.3708 | 0.13 | 0.0052 | −0.49 |
| | Cgrrf1 | cell growth regulator with ring finger domain 1 | 68755 | 0.2806 | 0.19 | 0.0018 | 0.64 |
| | Chchd5 | coiled-coil-helix-coiled-coil-helix domain containing 5 | 66170 | 0.0061 | 0.53 | 0.0261 | 0.32 |
| | Chuk | conserved helix-loop-helix ubiquitous kinase | 12675 | 0.0781 | −0.35 | 0.0046 | −0.43 |
| | Ciapin1 | cytokine induced apoptosis inhibitor 1 | 109006 | 0.2806 | 0.16 | 0.0000 | 0.86 |
| | Cib3 | calcium and integrin binding family member 3 | 234421 | 0.5817 | −0.30 | 0.0046 | −0.70 |
| | Ckb | creatine kinase, brain | 12709 | 0.5817 | 0.12 | 0.0019 | 1.08 |
| | Clic5 | chloride intracellular channel 5 | 224796 | 0.2038 | 0.25 | 0.0089 | −0.59 |
| | Clk1 | CDC-like kinase 1 | 12747 | 0.5817 | 0.21 | 0.0046 | 0.52 |
| | Clips | celipase, pancreatic | 109791 | 0.1412 | 1.62 | 0.0052 | 0.82 |
| | Cmtm8 | CKLF-like MARVEL transmembrane domain containing 8 | 70031 | 0.0000 | 0.57 | 0.1158 | 0.19 |
| | Cntfr | ciliary neurotrophic factor receptor | 12804 | 0.7187 | 0.01 | 0.0052 | 0.63 |
| | Cntnap2 | contactin associated protein-like 2 | 66797 | 0.6857 | 0.04 | 0.0000 | −0.94 |
| | Cntrob | centrobin, centrosomal BRCA2 interacting protein | 216846 | 0.0506 | −0.25 | 0.0028 | −0.37 |
| | Cobll1 | Cabl-like 1 | 319876 | 0.0091 | −0.36 | 0.1759 | −0.21 |
| | Col6a6 | collagen, type VI, alpha 6 | 245026 | 0.7476 | −0.07 | 0.0000 | −1.73 |
| | Commd7 | COMM domain containing 7 | 99311 | 0.0000 | −0.66 | 0.0000 | −0.76 |
| | Copa | coatomer protein complex subunit alpha | 12847 | 0.4615 | −0.20 | 0.0028 | −0.35 |
| | Coq9 | coenzyme Q9 homolog (yeast) | 67914 | 0.1412 | 0.22 | 0.0089 | 0.40 |
| | Cox18 | COX18 cytochrome c oxidase assembly | 231430 | 0.4615 | 0.08 | 0.0019 | 0.39 |
| | | homolog (S. cerevisiae) | | | | | |
| | Cox6a1 | cytochrome c oxidase, subunit VI a, polypeptide I | 12861 | 0.2806 | 0.21 | 0.0052 | 0.57 |
| | Cp | ceruloplasmin | 12870 | 0.0091 | 0.86 | 0.5595 | −0.14 |
| | Cpa2 | carboxypeptidase A2, pancreatic | 232680 | 0.2038 | 1.25 | 0.0046 | 0.65 |
| | Creb3 | cAMP responsive element binding protein 3 | 12913 | 0.3708 | 0.14 | 0.0052 | −0.36 |
| | Crebbp | CREB binding protein | 12914 | 0.0000 | −0.55 | 0.5446 | 0.04 |
| | Criml | cysteine rich transmembrane BMP regulator 1 (chordin | 50766 | 0.7187 | 0.03 | 0.0046 | −0.55 |
| | | like) | | | | | |
| | Crp | C-reactive protein, pentraxin-related | 12944 | 0.1412 | 0.50 | 0.0052 | 0.59 |
| | Crybg3 | beta-gamma crystallin domain containing 3 | 224273 | 0.0000 | −0.52 | 0.3561 | −0.20 |
| | Ctrc | chymotrypsin C (caldecrin) | 76701 | 0.2806 | 1.57 | 0.0000 | 4.58 |
| | Ctrl | chymotrypsin-like | 109660 | 0.1412 | 1.43 | 0.0052 | 0.47 |
| | Ctsk | cathepsin K | 13038 | 0.6857 | 0.04 | 0.0000 | −0.64 |
| | Ctss | cathepsin S | 13040 | 0.3708 | 0.27 | 0.0046 | −0.69 |
| | Cttnbp2 | cortactin binding protein 2 | 30785 | 0.2806 | 0.24 | 0.0000 | −0.80 |
| | Cutc | cutC copper transporter homolog (E. coli) | 66388 | 0.7476 | −0.09 | 0.0089 | −0.43 |
| | Cyp4f16 | cytochrome P450, family 4, subfamily f, polypeptide 16 | 70101 | 0.5817 | 0.09 | 0.0028 | −0.56 |
| | Cyp51 | cytochrome P450. family 51 | 13121 | 0.7476 | −0.13 | 0.0089 | 0.60 |
| | Cysltr2 | cysteinyl leukotriene receptor 2 | 70086 | 0.7658 | −0.02 | 0.0028 | −0.93 |
| | Cyyr1 | cysteine and tyrosine-rich protein 1 | 224405 | 0.2038 | −0.19 | 0.0089 | −0.41 |
| | D3Bwg0562e | DNA segment, Chr 3, Brigham &amp;&Women's Genetics | 229791 | 0.7187 | −0.14 | 0.0000 | −1.26 |
| | | 0562 expressed | | | | | |
| | D4Wsu53e | DNA segment, Chr 4, Wayne State University 53. | 27981 | 0.5817 | 0.33 | 0.0046 | 0.59 |
| | | expressed | | | | | |
| | Depl1 | death associated proteine-like 1 | 76747 | 0.0506 | −0.76 | 0.0000 | −1.10 |
| | Dapp1 | dual adaptor for phosphotyrosine and 3- | 26377 | 0.1056 | 0.56 | 0.0046 | 1.11 |
| | | phosphoinositides 1 | | | | | |
| | Dclk1 | doublecortin-like kinase 1 | 13175 | 0.7658 | −0.03 | 0.0028 | −0.45 |
| | Dcn | decorin | 13179 | 0.0000 | 1.83 | 0.0873 | 0.50 |
| | Defb1 | defensin beta 1 | 13214 | 0.7658 | −0.05 | 0.0000 | −0.93 |
| | Degs1 | degenerative spermatocyte homolog 1 (Drosophila) | 13244 | 0.4615 | 0.12 | 0.0028 | −0.58 |
| | Dgkb | diacylglycerol kinase, beta | 217480 | 0.7187 | −0.15 | 0.0046 | −0.55 |
| | Dgke | diacylglycerol kinase, epsilon | 56077 | 0.2806 | −0.28 | 0.0028 | −0.73 |
| | Dgkg | diacylglycerol kinase, gamma | 110197 | 0.0000 | −0.56 | 0.2533 | 0.14 |
| | Dhrs4 | dehydrogenase/reductase (SDR family) member 4 | 28200 | 0.1412 | 0.35 | 0.0019 | 0.75 |
| | Dhrs7b | dehydrogenase/reductase (SDR family) member 7B | 216820 | 0.0147 | 0.45 | 0.0089 | 0.39 |
| | Dio1 | deiodinase, iodothyronine, type 1 | 13370 | 0.0000 | −1.07 | 0.0000 | −1.45 |
| | Dip2b | DIP2 disco-interacting protein 2 homolog B | 239667 | 0.0000 | −0.50 | 0.5595 | −0.14 |
| | | (Drosophila) | | | | | |
| | Dlk1 | delta-like 1 homolog (Drosophila) | 13386 | 0.2806 | 0.54 | 0.0000 | 1.17 |
| | Dnahc9 | dynein, axonemal, heavy chain 9 | 237806 | 0.0781 | −0.36 | 0.0028 | −0.66 |
| | Dner | delta/notch-like EGF-related receptor | 227325 | 0.0506 | −0.54 | 0.0028 | −0.65 |
| | Dock10 | dedicator of cytokinesis 10 | 210293 | 0.0000 | −1.25 | 0.0028 | −1.22 |
| | Dpp7 | dipeptidylpeptidase 7 | 83768 | 0.0781 | 0.34 | 0.0000 | 0.78 |
| | Dpt | dermatopontin | 56429 | 0.0000 | 2.02 | 0.0089 | 1.14 |
| | Dusp18 | dual specificity phosphatase 18 | 75219 | 0.0147 | −0.72 | 0.0089 | −0.59 |
| | Dusp4 | dual specificity phosphatase 4 | 319520 | 0.0000 | −0.64 | 0.0000 | −0.75 |
| | Dync1h1 | dynein cytoplasmic 1 heavy chain 1 | 13424 | 0.0000 | −0.54 | 0.4909 | 0.06 |
| | Dzip1l | DAZ interacting protein 1-like | 72507 | 0.1412 | −0.19 | 0.0028 | −0.59 |
| | Eci1 | enoyl-Coenzyme A delta isomerase 1 | 13177 | 0.0091 | 0.32 | 0.1759 | 0.21 |
| | Efhc2 | EF-hand domain (C-terminal) containing 2 | 74405 | 0.7637 | −0.11 | 0.0028 | −0.71 |
| | Egfr | epidermal growth factor receptor | 13649 | 0.4615 | 0.21 | 0.0019 | 0.41 |
| | Ehd3 | EF-domain containing 3 | 57440 | 0.4615 | 0.12 | 0.0000 | −0.54 |
| | Eif4g3 | eukaryotic translation initiation factor 4 gamma, 3 | 230861 | 0.0091 | −0.52 | 0.5963 | −0.09 |
| | Elmod1 | ELMO domain containing 1 | 270162 | 0.7658 | −0.03 | 0.0000 | −0.82 |
| | Elof1 | elongation factor 1 homolog (ELF1, S. cerevisiae) | 66126 | 0.1056 | 0.31 | 0.0052 | −0.32 |
| | Emcn | endomucin | 59308 | 0.3708 | 0.24 | 0.0052 | −0.80 |
| | Eml1 | echinoderm microtubule associated protein like 1 | 68519 | 0.7658 | −0.01 | 0.0028 | −0.56 |
| | Eml6 | echinoderm microtubule associated protein like 6 | 237711 | 0.0506 | −0.64 | 0.0046 | −0.49 |
| | Eno1 | enolase 1, alpha non-neuron | 13806 | 0.4615 | 0.23 | 0.0000 | −0.82 |
| | Eno2 | enolase 2, gamma neuronal | 13807 | 0.5817 | 0.14 | 0.0019 | 0.74 |
| | Entpd3 | ectonucleoside triphosphate diphosphohydrolase 3 | 215449 | 0.0781 | −0.41 | 0.0089 | −0.36 |
| | Ep300 | EIA binding protein p300 | 328572 | 0.0061 | −0.61 | 0.6046 | −0.03 |
| | Epb4.1l4a | erythrocyte protein band 4.1-like 4a | 13824 | 0.7658 | −0.03 | 0.0089 | 0.43 |
| | Epm2a | epilepsy, progressive myoclonic epilepsy, type 2 gene | 13853 | 0.5817 | 0.12 | 0.0089 | 0.68 |
| | | alpha | | | | | |
| | Eps8l1 | EPS8-like 1 | 67425 | 0.3708 | 0.18 | 0.0046 | −0.52 |
| | Erap1 | endoplasmic reticulum aminopeptidase 1 | 80898 | 0.7476 | −0.13 | 0.0046 | −0.79 |
| | Etv1 | ets variant gene 1 | 14009 | 0.0061 | −0.35 | 0.3561 | 0.12 |
| | Exosc9 | exosome component 9 | 50911 | 0.7658 | −0.10 | 0.0028 | −0.95 |
| | Fabp4 | fatty acid binding protein 4, adipocyte | 11770 | 0.0091 | 0.95 | 0.3561 | 0.13 |
| | Fah | fumarylacetoacetate hydrolase | 14085 | 0.6857 | 0.08 | 0.0052 | 0.57 |
| | Fam107b | family with sequence similarity 107, member B | 66540 | 0.0147 | 1.06 | 0.0046 | 0.69 |
| | Fam122b | family with sequence similarity 122, member B | 78755 | 0.7658 | −0.07 | 0.0089 | −0.59 |
| | Fam158a | family with sequence similarity 158, member A | 85308 | 0.1056 | 0.29 | 0.0089 | 0.45 |
| | Fam163a | family with sequence similarity 163, member A | 329274 | 0.7187 | 0.04 | 0.0000 | 1.16 |
| | Fam171a1 | family with sequence similarity 171, member A1 | 269233 | 0.0091 | 0.65 | 0.0019 | 0.56 |
| | Fam171b | family with sequence similarity 171, member B | 241520 | 0.7658 | −0.07 | 0.0000 | −1.02 |
| | Fam183b | family with sequence similarity 183, member B | 75429 | 0.0000 | 0.81 | 0.5446 | 0.02 |
| | Fam193a | family with sequance similarity 193, member A | 231128 | 0.0000 | −0.62 | 0.5595 | −0.13 |
| | Fam20a | family with sequence similarity 20, member A | 208659 | 0.0061 | −0.59 | 0.0604 | −0.30 |
| | Fam38b | family with sequence similarity 38, member B | 667742 | 0.2806 | −0.23 | 0.0089 | −0.45 |
| | Fam43a | family with sequence similarity 43, member A | 224093 | 0.3708 | 0.20 | 0.0046 | −0.38 |
| | Fam55d | family with sequence similarity 55, member D | 244853 | 0.7187 | 0.05 | 0.0028 | −1.16 |
| | Fam64a | family with sequence similarity 64, member A | 109212 | 0.7658 | −0.12 | 0.0052 | −0.38 |
| | Fam70a | family with sequence similarity 70, member A | 245386 | 0.7658 | 0.00 | 0.0000 | −1.10 |
| | Fam81a | family with sequence similarity 81, member A | 76889 | 0.6857 | 0.07 | 0.0028 | −0.84 |
| | Farp1 | FERM, RhoGEF (Arhgef) and pleckstrin domain protein 1 | 223254 | 0.0000 | −0.60 | 0.5744 | −0.10 |
| | | (chondrocyte-derived) | | | | | |
| | Fat1 | FAT tumor suppressor homolog 1 (Drosophila) | 14107 | 0.0000 | −0.53 | 0.4909 | −0.18 |
| | Fbp2 | fructose bisphosphatase 2 | 14120 | 0.6857 | 0.05 | 0.0089 | 0.51 |
| | Fcer1g | Fc receptor, IgE, high affinity 1, gamma polypeptide | 14127 | 0.0781 | 0.33 | 0.0000 | −0.57 |
| | Fcgr4 | Fc receptor, IgG, low affinity IV | 246256 | 0.7187 | 0.01 | 0.0046 | −0.45 |
| | Fgf1 | fibroblast growth factor 1 | 14194 | 0.0091 | −0.41 | 0.0089 | −0.45 |
| | Fgf12 | fibroblast growth factor 12 | 14167 | 0.0147 | −0.41 | 0.0000 | −0.87 |
| | Filip1 | filamin A interacting protein 1 | 70598 | 0.6857 | 0.07 | 0.0052 | −0.57 |
| | Fkbp5 | FK506 binding protein 5 | 14229 | 0.6857 | −0.16 | 0.0028 | −0.55 |
| | Fmn2 | formin 2 | 54418 | 0.0506 | −0.53 | 0.0000 | −0.79 |
| | Fmo1 | flavin containing monooxygenase 1 | 14261 | 0.0324 | 0.38 | 0.0052 | 0.40 |
| | Fmo5 | flavin containing monooxygenase 5 | 14263 | 0.5817 | −0.19 | 0.0000 | −0.93 |
| | Fosb | FBJ osteosarcoma oncogene B | 14282 | 0.7569 | −0.25 | 0.0089 | 1.21 |
| | Foxn2 | forkhead box N2 | 14236 | 0.7187 | 0.03 | 0.0052 | −0.37 |
| | Frmd5 | FERM domain containing 5 | 228564 | 0.2038 | −0.34 | 0.0046 | −0.51 |
| | Fry | furry homolog (Drosophila) | 320365 | 0.0000 | −0.61 | 0.5446 | −0.15 |
| | Fto | fat mass and obesity associated | 26383 | 0.2806 | −0.20 | 0.0028 | −0.55 |
| | Fut10 | fucosyltransferase 10 | 171167 | 0.7187 | 0.02 | 0.0028 | −0.66 |
| | Fxyd3 | FXYD domain-containing ion transport regulator 3 | 17178 | 0.4615 | 0.09 | 0.0000 | −0.83 |
| | Fxyd6 | FXYD domain-containing ion transport regulator 6 | 59095 | 0.1059 | −0.27 | 0.0000 | −0.93 |
| | Galnt10 | UDP-N-acetyl-alpha-D-galactosamine:polypeptide N- | 171212 | 0.0217 | −0.48 | 0.0000 | −0.74 |
| | | acetylgalactosaminyltransferase 10 | | | | | |
| | Galnt12 | UDP-N-acetyl-alpha-D-galactosamine:polypeptide N- | 230145 | 0.6857 | 0.04 | 0.0089 | −0.37 |
| | | acetylgalactosaminyltransferase 12 | | | | | |
| | Galnt13 | UDP-N-acetyl-alpha-D-galactosamine:polypeptide N- | 271786 | 0.2038 | −0.29 | 0.0000 | 0.93 |
| | | acetylgalactosaminyltransferase 13 | | | | | |
| | Galnt4 | UDP-N-acetyl-alpha-D-galactosamine:polypeptide N- | 14426 | 0.1412 | −0.28 | 0.0000 | −0.60 |
| | | acetylgalactosaminyltransferase 4 | | | | | |
| | Galntl1 | UDP-N-acetyl-alpha-D-galactosamine:polypeptide N- | 108760 | 0.0091 | 0.44 | 0.3561 | 0.09 |
| | | acetylgalactosaminyltransferase-like 1 | | | | | |
| | Galntl4 | UDP-N-acetyl-alpha-D-galactosamine:polypeptide N- | 233733 | 0.7187 | 0.00 | 0.0052 | −0.48 |
| | | acetylgalactosaminyltransferase-like 4 | | | | | |
| | Gas2 | growth arrest specific 2 | 14453 | 0.7658 | −0.05 | 0.0000 | −0.93 |
| | Gatsl2 | GATS protein-like 2 | 80909 | 0.0091 | −0.65 | 0.6046 | −0.06 |
| | Gbp2 | guanylate binding protein 2 | 14469 | 0.0091 | 0.41 | 0.2533 | −0.35 |
| | Gcnt1 | glucosaminyl (N-acetyl) transferase 1, core 2 | 14537 | 0.0781 | 0.86 | 0.0046 | 1.03 |
| | Gcnt2 | glucosaminyl (N-acetyl) transferase 2, 1-branching | 14538 | 0.0506 | −0.40 | 0.0028 | −0.47 |
| | | enzyme | | | | | |
| | Gdap2 | ganglioside-induced differentiation-associated-protein 2 | 14547 | 0.7658 | −0.07 | 0.0000 | 0.71 |
| | Gem | GTP binding protein (gene overexpressed in skeletal | 14579 | 0.6857 | 0.05 | 0.0000 | −1.16 |
| | | muscle) | | | | | |
| | Gfra3 | glial cell line derived neurotrophic factor family | 14587 | 0.2038 | 0.50 | 0.0089 | −0.62 |
| | | receptor alpha 3 | | | | | |
| | Ggcx | gamma-glutamyl carboxylase | 56316 | 0.7187 | 0.04 | 0.0000 | −0.87 |
| | Ghr1 | ghrelin | 58991 | 0.6857 | 0.13 | 0.0046 | −0.85 |
| | Gipc1 | GIPC PDZ domain containing family, member 1 | 67903 | 0.2038 | 0.22 | 0.0046 | 0.33 |
| | Gipc2 | GIPC PDZ domain containing family, member 2 | 54120 | 0.5817 | −0.17 | 0.0000 | −0.51 |
| | Gib1 | galactosidase, beta 1 | 12091 | 0.6857 | 0.04 | 0.0000 | −0.81 |
| | Glb1l2 | galactosidase, beta 1-like 2 | 244757 | 0.7658 | −0.09 | 0.0000 | −0.77 |
| | Glo1 | glyoxalase 1 | 109801 | 0.6857 | 0.03 | 0.0000 | 0.82 |
| | Glra1 | glycine receptor, alpha 1 subunit | 14654 | 0.0091 | 0.67 | 0.0000 | −0.82 |
| | Glrb | glycine receptor, beta subunit | 14658 | 0.3708 | 0.25 | 0.0052 | −0.37 |
| | Gls2 | glutaminase 2 (liver, mitochondrial) | 216456 | 0.0781 | 0.77 | 0.0019 | 0.88 |
| | Gm10260 | predicted gene 10260 | 100039740 | 0.2038 | 0.28 | 0.0000 | 0.61 |
| | Gm11942 | predicted gene 11942 | 665298 | 0.2806 | 0.44 | 0.0000 | 1.38 |
| | Gm14085 | predicted gene 14085 | 381417 | 0.0217 | −0.34 | 0.0046 | −0.59 |
| | Gm14420 | predicted gene 14420 | 628308 | 0.7187 | 0.01 | 0.0000 | 0.73 |
| | Gm15800 | predicted gene 15800 | 269700 | 0.0000 | −0.63 | 0.6046 | −0.03 |
| | Gm340 | predicted gene 340 | 381224 | 0.0000 | −0.64 | 0.1759 | −0.25 |
| | Gm3468 | predicted gene 3468 | 100503971 | 0.4615 | −0.26 | 0.0089 | −1.16 |
| | Gm5114 | predicted gene 5114 | 330513 | 0.3708 | 0.19 | 0.0019 | 0.57 |
| | Gm6404 | predicted gene 6404 | 623174 | 0.3708 | 0.29 | 0.0046 | 0.70 |
| | Gm6969 | predicted pseudogene 6969 | 629383 | 0.0217 | 0.52 | 0.0000 | 2.65 |
| | Gm7582 | predicted gene 7582 | 665317 | 0.0324 | −0.63 | 0.0089 | −1.00 |
| | Gm9292 | predicted gene 9292 | 668662 | 0.6857 | 0.08 | 0.0000 | 0.96 |
| | Gmnn | geminin | 57441 | 0.7658 | −0.06 | 0.0046 | −0.35 |
| | Gmpr | guanosine monophosphate reductase | 66355 | 0.5817 | 0.14 | 0.0000 | −1.18 |
| | Gnao1 | guanine nucleotide binding protein, alpha D | 14681 | 0.0000 | −0.58 | 0.6046 | −0.06 |
| | Gnat2 | guanine nucleotide binding protein, alpha transducing 2 | 14686 | 0.6857 | 0.07 | 0.0028 | −0.47 |
| | Golm1 | golgi membrane protein 1 | 105348 | 0.7658 | −0.01 | 0.0089 | −0.45 |
| | Gpa33 | glycoprotein A33 (transmembrane) | 59290 | 0.7658 | −0.05 | 0.0028 | −0.49 |
| | Gpld1 | glycosylphosphatidylinositol specific phospholipase D1 | 14756 | 0.0217 | −0.78 | 0.0028 | −0.90 |
| | Gpm6a | glycoprotein m6a | 234267 | 0.0000 | 0.74 | 0.0046 | 0.43 |
| | Gpr119 | G protein-coupled receptor 116 | 224792 | 0.7658 | −0.09 | 0.0028 | −0.80 |
| | Gpr157 | G protein-coupled receptor 157 | 269604 | 0.2806 | 0.21 | 0.0052 | 0.36 |
| | Gpr179 | G protein-coupled receptor 179 | 217143 | 0.2038 | −0.24 | 0.0019 | 0.56 |
| | Gpr19 | G protein-coupled receptor 19 | 14760 | 0.2038 | 0.27 | 0.0046 | 0.51 |
| | Gramd1b | GRAM domain containing 1B | 235283 | 0.0147 | −0.35 | 0.0052 | −0.37 |
| | Gsta4 | glutathione S-transferase, alpha 4 | 14860 | 0.2038 | 0.38 | 0.0089 | 0.69 |
| | Gucyla3 | guanylate cyclase 1, soluble, alpha 3 | 60596 | 0.6857 | 0.14 | 0.0089 | −0.63 |
| | Gucy2c | guanylate cyclase 2c | 14917 | 0.2806 | −0.36 | 0.0000 | −1.20 |
| | H19 | H19 fetal liver mRNA | 14955 | 0.0506 | 1.37 | 0.0089 | 0.81 |
| | H2-Aa | histocompatibility 2, class II antigen A, alpha | 14960 | 0.5817 | −0.17 | 0.0000 | −1.33 |
| | H2-Ab1 | histocompatibility 2, class II antigen A, beta 1 | 14961 | 0.7187 | 0.02 | 0.0000 | −1.72 |
| | H2afz | H2A histone family, member Z | 51788 | 0.7658 | −0.11 | 0.0000 | −4.41 |
| | H2-Eb1 | histocompatibility 2, class II antigen E beta | 14969 | 0.7187 | −0.16 | 0.0000 | −0.99 |
| | H2-K1 | histocompatibility 2, KI, K region | 14972 | 0.7187 | 0.02 | 0.0000 | −1.93 |
| | H2-K2 | histocompatibility 2, K region locus 2 | 630499 | 0.6857 | 0.04 | 0.0028 | −0.57 |
| | H2-Ke6 | H2-K region expressed gene 6 | 14979 | 0.2806 | 0.28 | 0.0019 | 0.66 |
| | H2-T22 | histocompatibility 2, T region locus 22 | 15039 | 0.5817 | −0.16 | 0.0000 | −2.87 |
| | H2-T23 | histocompatibility 2, T region locus 23 | 15040 | 0.5817 | 0.17 | 0.0000 | −1.55 |
| | H2-T24 | histocompatibility 2, T region locus 24 | 15042 | 0.0217 | 0.34 | 0.0000 | 0.48 |
| | Hapln1 | hyaluronan and proteoglycan link protein 1 | 12950 | 0.7187 | 0.02 | 0.0000 | −0.70 |
| | HbegF | heparin-binding EGF-like growth factor | 15200 | 0.1412 | −0.23 | 0.0000 | −0.48 |
| | Hddc3 | HD domain containing 3 | 68695 | 0.2806 | 0.28 | 0.0000 | 0.73 |
| | Hdhd3 | haloacid dehalogenase-like hydrolase domain | 72748 | 0.1412 | 0.26 | 0.0046 | 0.57 |
| | | containing 3 | | | | | |
| | Heatr8 | HEAT repeat containing 8 | 381538 | 0.0061 | −0.42 | 0.6046 | −0.01 |
| | Hebp1 | heme binding protein 1 | 15199 | 0.4615 | 0.18 | 0.0000 | 0.68 |
| | Heg1 | HEG homolog 1 (zebrafish) | 77446 | 0.0000 | −0.47 | 0.5744 | −0.13 |
| | Hemk1 | HemK methyltransferase family member 1 | 69536 | 0.4615 | 0.10 | 0.0019 | 0.57 |
| | Herc1 | hect (homologous to the E6-AP (LI8E3A) carboxyl | 235439 | 0.0091 | −0.57 | 0.6046 | −0.07 |
| | | terminus) domain and RCC1 (CHC1)-like domain (RLD) 1 | | | | | |
| | Hgfac | hepatocyte growth factor activator | 54426 | 0.3708 | 0.20 | 0.0028 | −0.77 |
| | Hgsnat | heparan-alpha-glucosaminide N-acetyltransferase | 52120 | 0.7187 | 0.03 | 0.0052 | −0.39 |
| | Hipk3 | homeodomain interacting protein kinase 3 | 15259 | 0.0000 | −0.57 | 0.2533 | −0.19 |
| | Hist1h1a | histone cluster 1, H1a | 80838 | 0.4615 | 0.28 | 0.0000 | 0.83 |
| | Hist1h2bg | histone cluster 1, H2bg | 319181 | 0.7658 | −0.04 | 0.0046 | −0.59 |
| | Hist1h2bm | histone cluster 1, H2bm | 319186 | 0.2038 | 0.38 | 0.0052 | 0.59 |
| | Hist1h4i | histone cluster 1, H4i | 319158 | 0.2038 | 0.40 | 0.0000 | 0.82 |
| | Hist2h2bb | histone cluster 2, H2bb | 319189 | 0.6857 | 0.14 | 0.0052 | 0.86 |
| | Hivep1 | human immunodeficiency virus type 1 enhancer binding | 110521 | 0.0000 | −0.53 | 0.0387 | −0.34 |
| | | protein 1 | | | | | |
| | Hivep2 | human immunodeficiency virus type 1 enhancer binding | 15273 | 0.0324 | −0.38 | 0.0046 | −0.61 |
| | | protein 2 | | | | | |
| | Hjurp | Holliday junction recognition protein | 381280 | 0.1056 | −0.43 | 0.0000 | −1.17 |
| | Hmgcll1 | 3-hydroxymethyl-3-methylglutaryl-Coenzyme A lyase- | 208982 | 0.7569 | −0.13 | 0.0052 | −0.66 |
| | | like 1 | | | | | |
| | Hmgn2- | high mobility group nucleosomal binding domain 2, | 100039489 | 0.0506 | −0.54 | 0.0000 | −0.88 |
| | ps1 | pseudogene 1 | | | | | |
| | Hmox1 | heme oxygenase (decycling) 1 | 15368 | 0.0147 | 0.54 | 0.0052 | 0.46 |
| | Hpgd | hydroxyprostaglandin dehydrogenase 15 (NAD) | 15446 | 0.2806 | 0.25 | 0.0046 | −0.79 |
| | Hrsp12 | heat-responsive protein 12 | 15473 | 0.0506 | 0.59 | 0.0052 | 0.77 |
| | Hsd17b10 | hydroxysteroid (17-beta) dehydrogenase 10 | 15108 | 0.0061 | 0.76 | 0.4909 | 0.07 |
| | Hspa14 | heat shock protein 14 | 50497 | 0.0000 | 0.97 | 0.0000 | 0.93 |
| | Hspa8 | heat shock protein 8 | 15481 | 0.2038 | 0.29 | 0.0000 | −1.63 |
| | Htr3a | 5-hydroxytryptamine (serotonin) receptor 3A | 15561 | 0.5817 | 0.13 | 0.0028 | −1.08 |
| | Hunk | hormonally upregulated Neu-associated kinase | 26559 | 0.7658 | −0.02 | 0.0000 | −0.93 |
| | Huwe1 | HECT, UBA and WWE domain containing 1 | 59026 | 0.0091 | −0.58 | 0.6046 | −0.07 |
| | Hyi | hydroxypyruvate isomerase homolog (E. coli) | 68180 | 0.6857 | 0.06 | 0.0000 | 0.96 |
| | Icall | islet call autoantigen 1-like | 70375 | 0.5817 | −0.22 | 0.0000 | 1.83 |
| | Idua | iduronidase, algha-L- | 15932 | 0.4615 | 0.09 | 0.0028 | −0.49 |
| | Ier2 | immediate early response 2 | 15936 | 0.2806 | 0.44 | 0.0019 | 1.20 |
| | Ifi44 | interferon-induced protein 44 | 99899 | 0.6857 | −0.11 | 0.0000 | −0.97 |
| | Ifih1 | interferon induced with helicase C domain 1 | 71586 | 0.6857 | −0.18 | 0.0000 | −1.47 |
| | Ifitm1 | interferon induced transmembrane protein 1 | 68713 | 0.0091 | 0.57 | 0.0873 | 0.19 |
| | Ikbip | IKBKB interacting protein | 67454 | 0.7350 | −0.11 | 0.0000 | −0.60 |
| | Il13ra1 | interleukin 13 receptor, alpha 1 | 16164 | 0.5817 | 0.10 | 0.0052 | −0.62 |
| | Il6ra | interleukin 6 receptor, alpha | 16194 | 0.3708 | −0.30 | 0.0052 | −0.46 |
| | Ino80 | INO80 homolog (S. cerevisiae) | 68142 | 0.0061 | −0.54 | 0.6046 | −0.04 |
| | Ino80d | INO80 complex subunit D | 227195 | 0.0091 | −0.41 | 0.3561 | 0.13 |
| | Inpp5b | inositol polyphosphate-5-phosphatase 8 | 16330 | 0.7637 | −0.04 | 0.0089 | −0.28 |
| | Iqgap1 | IQ motif containing GTPase activating protein 1 | 29875 | 0.0147 | −0.42 | 0.0089 | −0.37 |
| | Irak1bp1 | interleukin-1 receptor-associated kinase 1 binding | 65099 | 0.5817 | 0.08 | 0.0000 | −1.32 |
| | | protein 1 | | | | | |
| | Irgm2 | immunity-related GTPase family M member 2 | 54396 | 0.7658 | −0.09 | 0.0000 | −0.72 |
| | Itfg3 | integrin alpha FG-GAP repeat containing 3 | 106581 | 0.7658 | −0.06 | 0.0000 | −0.55 |
| | Itga7 | integrin alpha 7 | 16404 | 0.4615 | 0.18 | 0.0046 | −0.37 |
| | Itgax | integrin alpha X | 16411 | 0.2038 | −0.27 | 0.0089 | −0.42 |
| | Itih1 | inter-alpha trypsin inhibitor, heavy chain 1 | 16424 | 0.3708 | −0.19 | 0.0000 | 1.07 |
| | Itpr2 | inositol 1,4,5-triphosphate receptor 2 | 16439 | 0.4615 | −0.19 | 0.0000 | −0.66 |
| | Ivd | isovaleryl coenzyme A dehydrogenase | 56357 | 0.1412 | 0.24 | 0.0028 | −0.37 |
| | Jakmip1 | janus kinase and microtubule interacting protein 1 | 76071 | 0.6857 | 0.04 | 0.0000 | −0.86 |
| | Jam2 | junction adhesion molecule 2 | 67374 | 0.2038 | 0.36 | 0.0052 | −0.80 |
| | Jmjd5 | jumonji domain containing 5 | 77035 | 0.0217 | −0.56 | 0.0028 | −0.56 |
| | Jun | Jun oncogene | 16476 | 0.3708 | 0.36 | 0.0000 | 1.22 |
| | Junb | Jun-B oncogene | 16477 | 0.3708 | 0.19 | 0.0046 | 0.66 |
| | Kank1 | KN motif and ankyrin repeat domains 1 | 107351 | 0.0781 | −0.37 | 0.0046 | −0.47 |
| | Kcnab3 | potassium voltage-gated channel, shaker-related | 16499 | 0.0506 | −0.47 | 0.0028 | −0.61 |
| | | subfamily, beta member 3 | | | | | |
| | Kcne3 | potassium voltage-gated channel, Isk-related | 57442 | 0.5817 | 0.11 | 0.0000 | −0.52 |
| | | subfamily, gene 3 | | | | | |
| | Kcnh6 | potassium voltage-gated channel, subfamily H (eag- | 192775 | 0.0000 | −0.50 | 0.5446 | 0.01 |
| | | related), member 6 | | | | | |
| | Kcnh8 | potassium voltage-gated channel, subfamily H (eag- | 211468 | 0.0147 | −0.38 | 0.0000 | −0.42 |
| | | related), member 8 | | | | | |
| | Kcnip1 | Kv channel-interacting protein 1 | 70357 | 0.0217 | −0.37 | 0.0000 | −0.74 |
| | Kcnip4 | Kv channel interacting protein 4 | 80334 | 0.6857 | 0.06 | 0.0028 | −1.00 |
| | Kcnj13 | potassium inwardly-rectifying channel, subfamily J, | 100040591 | 0.5817 | 0.11 | 0.0052 | −0.69 |
| | | member 13 | | | | | |
| | Kcnj6 | potassium inwardly-rectifying channel, subfamily J, | 16522 | 0.0781 | −0.33 | 0.0089 | −0.27 |
| | | member 6 | | | | | |
| | Kcnma1 | potassium large conductance calcium-activated | 16531 | 0.0091 | −0.62 | 0.0387 | −0.32 |
| | | channel, subfamily M, alpha member 1 | | | | | |
| | Kcnn3 | potassium intermediate/small conductance calcium- | 140493 | 0.0324 | −0.50 | 0.0052 | −0.50 |
| | | activated channel, subfamily N, member 3 | | | | | |
| | Kif23 | kinesin family member 23 | 71819 | 0.4615 | −0.36 | 0.0052 | −0.56 |
| | Kif4 | kinesin family member 4 | 16571 | 0.3708 | −0.40 | 0.0028 | −0.61 |
| | Kit | kit oncogene | 16590 | 0.7658 | −0.04 | 0.0052 | −0.33 |
| | Klf9 | Kruppel-like factor 9 | 16601 | 0.6857 | 0.04 | 0.0052 | −0.44 |
| | Klhdc4 | kelch domain containing 4 | 234825 | 0.2806 | −0.22 | 0.0046 | −0.46 |
| | klhdc5 | kelch domain containing 5 | 232539 | 0.7658 | −0.04 | 0.0028 | −0.53 |
| | Klhl1 | kelch-like 1 (Drosophila) | 93688 | 0.0061 | 0.40 | 0.0019 | 0.40 |
| | Klhl33 | kelch-like 33 (Drosophila) | 546611 | 0.7658 | −0.08 | 0.0019 | 0.87 |
| | Kras | v-Ki-ras2 Kirsten rat sarcoma viral oncogene homolog | 16653 | 0.4615 | −0.21 | 0.0089 | −0.50 |
| | Lancl3 | LanC lantibiotic synthetase component C-like 3 | 236285 | 0.6857 | −0.16 | 0.0052 | −0.47 |
| | | (bacterial) | | | | | |
| | Laptm5 | lysosomal-associated protein transmembrane 5 | 16792 | 0.0091 | 0.74 | 0.5963 | −0.07 |
| | Lbp | lipopolysaccharide binding protein | 16803 | 0.0000 | 1.41 | 0.0000 | 1.03 |
| | Ldlrad3 | low density lipoprotein receptor class A domain | 241576 | 0.1056 | −0.22 | 0.0052 | −0.33 |
| | | containing 3 | | | | | |
| | Lgi1 | leucine-rich repeat LG1 family, member 1 | 56839 | 0.0781 | −0.74 | 0.0000 | −1.35 |
| | Limch1 | LIM and calponin homology domains 1 | 77569 | 0.7658 | −0.04 | 0.0089 | −0.62 |
| | Lims2 | LIM and senescent cell antigen like domains 2 | 225341 | 0.3708 | 0.22 | 0.0089 | −0.35 |
| | Lixll | Lixl-like | 280411 | 0.7658 | −0.08 | 0.0046 | −0.67 |
| | Lpgat1 | lysophosphatidylglycerol acyltransferase 1 | 226856 | 0.2038 | −0.29 | 0.0089 | −0.50 |
| | Lpl | lipoprotein lipase | 16956 | 0.4615 | 0.19 | 0.0000 | 0.97 |
| | Lrp8 | low density lipoprotein receptor-related protein 8, | 16975 | 0.0000 | −1.42 | 0.0000 | −0.98 |
| | | apolipoprotein e receptor | | | | | |
| | Lrrc1 | leucine rich repeat containing 1 | 214345 | 0.7658 | −0.06 | 0.0046 | −0.75 |
| | Lrrc55 | leucine rich repeat containing 55 | 241528 | 0.7658 | −0.07 | 0.0000 | −0.74 |
| | Lrrk2 | leucine-rich repeat kinase 2 | 66725 | 0.6857 | 0.03 | 0.0089 | −0.34 |
| | Lrrn1 | leucine rich repeat protein 1, neuronal | 16979 | 0.1412 | −0.37 | 0.0000 | −0.69 |
| | Lrrtm4 | leucine rich repeat transmembrane neuronal 4 | 243499 | 0.1412 | 0.41 | 0.0046 | −0.81 |
| | Ltbp4 | latent transforming growth factor beta binding protein 4 | 108075 | 0.5817 | 0.17 | 0.0089 | −0.77 |
| | Lum | lumican | 17022 | 0.0000 | 1.20 | 0.6046 | −0.10 |
| | Luzp2 | leucine zipper protein 2 | 233271 | 0.2038 | 0.44 | 0.0019 | 1.18 |
| | Ly6a | lymphocyte antigen 6 complex, locus A | 110454 | 0.0506 | 0.97 | 0.0046 | −1.09 |
| | Ly6c1 | lymphocyte antigen 6 complex, locus C1 | 17067 | 0.0217 | 0.82 | 0.0000 | −0.90 |
| | Ly6e | lymphocyte antigen 6 complex, locus E | 17069 | 0.4615 | 0.14 | 0.0000 | −0.95 |
| | Ly96 | lymphocyte antigen 96 | 17087 | 0.6857 | 0.14 | 0.0028 | −1.33 |
| | Lyrm7 | LYR motif containing 7 | 75530 | 0.0000 | −1.09 | 0.0046 | −0.53 |
| | Lyve1 | lymphatic vessel endothelial hyaluronan receptor 1 | 114332 | 0.2038 | −0.50 | 0.0000 | −1.08 |
| | Lyz2 | lysozyme 2 | 17105 | 0.0091 | 1.34 | 0.2533 | 0.37 |
| | Macf1 | microtubule-actin crosslinking factor 1 | 11426 | 0.0000 | −0.61 | 0.4909 | −0.18 |
| | Macrod2 | MACRO domain containing 2 | 72899 | 0.0147 | −0.38 | 0.0046 | −0.43 |
| | Man2b1 | mannosidase 2, alpha B1 | 17159 | 0.6857 | 0.09 | 0.0000 | −0.83 |
| | Map3k5 | mitogen-activated protein kinas kinase kinase 5 | 26408 | 0.1412 | −0.34 | 0.0028 | −0.52 |
| | Marveld2 | MARVEL (membrane-associating) domain containing 2 | 218518 | 0.5817 | −0.14 | 0.0028 | −0.38 |
| | Matn2 | matrilin 2 | 17181 | 0.0506 | 0.44 | 0.0000 | −0.85 |
| | Mccc2 | methylcrotonoyl-Coenzyme A carboxylase 2 (beta) | 78038 | 0.1056 | 0.26 | 0.0019 | 0.41 |
| | Mcee | methylmalonyl CoA epimerase | 73724 | 0.0061 | 0.60 | 0.4909 | −0.15 |
| | Mctp2 | multiple C2 domains, transmembrane 2 | 244049 | 0.7569 | −0.11 | 0.0000 | −0.92 |
| | Melk | maternal embryonic leucine zipper kinase | 17279 | 0.7187 | −0.19 | 0.0052 | −0.49 |
| | Meox1 | mesenchyme homeobox 1 | 17285 | 0.3708 | 0.24 | 0.0000 | −0.81 |
| | Meox2 | mesenchyme homeobox 2 | 17286 | 0.4615 | 0.23 | 0.0052 | −0.57 |
| | Metap1d | methionyl aminopeptidase type 1D (mitochondrial) | 66559 | 0.5817 | 0.06 | 0.0000 | −0.81 |
| | Mfap4 | microfbrillar-associated protein 4 | 76293 | 0.2038 | 0.51 | 0.0028 | −0.72 |
| | Mgam | maltase-glucoamylase | 232714 | 0.7658 | −0.05 | 0.0000 | 1.00 |
| | Mgat3 | mannoside acetylglucosaminyltransferase 3 | 17309 | 0.7350 | −0.15 | 0.0028 | −0.59 |
| | Mgp | matrix G1a protein | 17313 | 0.0506 | 0.57 | 0.0089 | −0.45 |
| | Mib1 | mindbomb homolog 1 (Drosophila) | 225164 | 0.0091 | −0.41 | 0.5872 | −0.08 |
| | Mical2 | microtubule associated monoxygenase, calponin and | 320878 | 0.0000 | −0.46 | 0.5446 | −0.12 |
| | | LIM domain containing 2 | | | | | |
| | Mical3 | microtubule associated monoxygenase, calponin and | 194401 | 0.0781 | −0.52 | 0.0000 | −0.76 |
| | | LIM domain containing 3 | | | | | |
| | Mink1 | misshapen-like kinase 1 (zebrafish) | 50932 | 0.0091 | −0.53 | 0.6046 | −0.04 |
| | Mir679 | microRNA 679 | 751539 | 0.2038 | −0.43 | 0.0019 | 0.61 |
| | Mis12 | MIS12 homolog (yeast) | 67139 | 0.0000 | 1.03 | 0.2533 | 0.18 |
| | Mll2 | myeloid/lymphoid or mixed-lineage leukemia 2 | 381022 | 0.0000 | −0.60 | 0.4909 | 0.06 |
| | Mlxip | MLX interacting protein | 208104 | 0.0061 | −0.43 | 0.4909 | 0.06 |
| | Mpeg1 | macrophage expressed gene 1 | 17476 | 0.4615 | 0.20 | 0.0000 | −1.06 |
| | Mpp6 | membrane protein, palmitoylated 6 (MAGUK p55 | 56524 | 0.7658 | −0.03 | 0.0000 | 0.60 |
| | | subfamily member 6) | | | | | |
| | Mril | methylthioribose-1-phosphate isomerase | 67873 | 0.3708 | 0.14 | 0.0089 | −0.34 |
| | | homolog (S. cerevisiae) | | | | | |
| | Mrpl20 | mitochondrial ribosomal protein L20 | 66448 | 0.2806 | 0.29 | 0.0000 | 1.40 |
| | Mrpl35 | mitochondrial ribosomal protein L35 | 66223 | 0.0061 | 0.53 | 0.6046 | 0.00 |
| | Mrps18a | mitochondrial ribosomal protein S18A | 68565 | 0.7637 | −0.08 | 0.0052 | 0.45 |
| | Mrs2 | MRS2 magnesium homeostasis factor humolog | 380836 | 0.2806 | −0.26 | 0.0028 | −0.52 |
| | | (S. cerevisiae) | | | | | |
| | Msln | mesothelin | 56047 | 0.0324 | −0.76 | 0.0052 | −0.54 |
| | Mslnl | mesothelin-like | 328783 | 0.4615 | −0.34 | 0.0000 | −0.64 |
| | Mt1 | metallothionein 1 | 17748 | 0.0147 | 0.87 | 0.0089 | 0.56 |
| | Mt2 | metallothionein 2 | 17750 | 0.0091 | 1.14 | 0.0000 | 1.48 |
| | Mtmr11 | myotubolarin related protein 11 | 194126 | 0.7658 | −0.04 | 0.0000 | −1.03 |
| | Muc4 | mucin 4 | 140474 | 0.0000 | −0.93 | 0.0000 | −0.74 |
| | Myo3a | myosin IIIA | 667663 | 0.1412 | 0.74 | 0.0000 | 1.75 |
| | Nacc1 | nucleus accumbens associated 1, BEN and BTB (POZ) | 66830 | 0.0061 | −0.52 | 0.2533 | −0.28 |
| | | domain containing | | | | | |
| | Napepld | N-acyl phosphatidylethanolamina phospholipase D | 242864 | 0.5817 | 0.11 | 0.0000 | −0.96 |
| | Naprt1 | nicotinate phosphoribosyltransferase domain | 223646 | 0.2806 | 0.35 | 0.0052 | 0.71 |
| | | containing 1 | | | | | |
| | Nbea | neurobeachin | 26422 | 0.0061 | −0.59 | 0.5446 | 0.02 |
| | Ncam1 | neural cell adhesion molecule 1 | 17967 | 0.1056 | −0.28 | 0.0046 | −0.41 |
| | Ncapd2 | non-SMC condensin 1 complex, subunit D2 | 68298 | 0.2038 | −0.36 | 0.0028 | −0.53 |
| | Ncoa6 | nuclear receptor coactivator 6 | 56406 | 0.0061 | −0.58 | 0.5595 | −0.16 |
| | Ncstn | nicastrin | 59287 | 0.7350 | −0.13 | 0.0028 | −0.37 |
| | Ndufa4l2 | NADH dehydrogenase (ubiquinone) 1 alpha subcomplex, | 407790 | 0.0147 | 0.53 | 0.0089 | −0.39 |
| | | 4-like 2 | | | | | |
| | Ndufaf1 | NADH dehydragenase (ubiquinone) 1 alpha subcomplex, | 69702 | 0.7476 | −0.07 | 0.0028 | −0.47 |
| | | assembly factor 1 | | | | | |
| | Ndufc1 | NADH dehydrogenase (ubiquinone) 1, subcomplex | 66377 | 0.0091 | 0.48 | 0.3561 | 0.09 |
| | | unknown, 1 | | | | | |
| | Ndufs5 | NADH dehydrogenase (ubiquinone) Fe—S protein 5 | 595136 | 0.7658 | −0.05 | 0.0000 | −1.53 |
| | Nebl | nebulette | 74103 | 0.0061 | 1.05 | 0.2533 | 0.31 |
| | Necab1 | N-terminal EF-hand calcium binding protein 1 | 69352 | 0.6857 | 0.04 | 0.0028 | −0.57 |
| | Nedd1 | neural precursor cell expressed, developmentally | 17997 | 0.5817 | 0.10 | 0.0089 | −0.39 |
| | | down-regulated gene 1 | | | | | |
| | Nefm | neurofilament, medium polypeptide | 18040 | 0.3708 | −0.25 | 0.0052 | −0.48 |
| | Nell1 | NEL-like 1 (chicken) | 338352 | 0.7658 | −0.05 | 0.0089 | −0.32 |
| | Nell2 | NEL-like 2 (chicken) | 54003 | 0.7658 | −0.04 | 0.0028 | −0.37 |
| | Neurl1b | neuralized homolog 1b (Drosophila) | 240055 | 0.2806 | −0.29 | 0.0028 | −0.47 |
| | NfI | neurofibromatosis I | 18015 | 0.0000 | −0.75 | 0.5595 | −0.13 |
| | Nfasc | neurofascin | 269116 | 0.0091 | −0.51 | 0.6046 | −0.02 |
| | Nfix | nuclear factor I/X | 18032 | 0.7658 | −0.03 | 0.0052 | 0.58 |
| | Nfs1 | nitrogen fixation gene 1 (S. cerevisiae) | 18041 | 0.0000 | 0.93 | 0.0019 | 0.64 |
| | Nhej1 | nonhomologous end-joining factor 1 | 75570 | 0.0217 | 0.37 | 0.0089 | 0.45 |
| | Nipal1 | NIPA-like domain containing 1 | 70701 | 0.1412 | −0.43 | 0.0000 | −1.11 |
| | Nkg7 | natural killer cell group 7 sequence | 72310 | 0.1056 | 0.17 | 0.0089 | 0.37 |
| | Nme3 | non-metastatic cells 3, protein expressed in | 79059 | 0.0000 | 0.49 | 0.0604 | 0.38 |
| | Nnt | nicotinamide nucleotide transhydrogenase | 18115 | 0.5817 | 0.07 | 0.0000 | 1.27 |
| | Nop10 | NOP10 ribonucleoprotein homolog (yeast) | 66181 | 0.0091 | 0.62 | 0.0604 | −0.33 |
| | Npl | N-acetylneuraminate pyruvate lyase | 74091 | 0.2806 | 0.31 | 0.0019 | 0.98 |
| | Npr1 | natriuretic peptide receptor 1 | 18160 | 0.1412 | 0.37 | 0.0000 | 0.45 |
| | Npy | neuropeptide Y | 109648 | 0.6857 | 0.06 | 0.0019 | 0.82 |
| | Nr1h4 | nuclear receptor subfamily 1, group H, member 4 | 20186 | 0.6857 | −0.23 | 0.0028 | −1.00 |
| | Nrsn1 | neurensin 1 | 22360 | 0.5817 | 0.15 | 0.0052 | −0.83 |
| | Nsd1 | nuclear receptor-binding SET-domain protein 1 | 18193 | 0.0000 | −0.57 | 0.5744 | −0.09 |
| | Nup210 | nucleoporin 210 | 54563 | 0.0061 | −0.41 | 0.0089 | −0.46 |
| | Nup214 | nucleoporin 214 | 227720 | 0.0000 | −0.50 | 0.5595 | −0.13 |
| | Oaf | OAF homolog (Drosophila) | 102644 | 0.4615 | 0.15 | 0.0046 | −0.57 |
| | Olfm3 | olfactomedin 3 | 229759 | 0.3708 | 0.13 | 0.0028 | −1.05 |
| | Olfr558 | olfactory receptor 558 | 259097 | 0.6857 | 0.07 | 0.0052 | −0.80 |
| | Olfr723 | olfactory receptor 723 | 259147 | 0.0781 | −0.25 | 0.0000 | −0.48 |
| | Osbpl3 | oxysterol binding protein-like 3 | 71720 | 0.0781 | −0.32 | 0.0052 | −0.42 |
| | Oxr1 | oxidation resistance 1 | 170719 | 0.7658 | −0.05 | 0.0028 | −0.57 |
| | Oxsm | 3-oxoacyl-ACP synthase, mitochondrial | 71147 | 0.3708 | 0.27 | 0.0089 | −0.58 |
| | P2rx4 | purinergic receptor P2X, ligand-gated ion channel 4 | 18438 | 0.4615 | 0.16 | 0.0046 | −0.59 |
| | P2ryl | purinergic receptor P2Y, G-protein coupled 1 | 18441 | 0.4615 | −0.28 | 0.0046 | −0.54 |
| | P4ha2 | procollagen-proline, 2-oxoglutarate, 4-dioxygenase | 18452 | 0.0506 | 0.62 | 0.0046 | 0.61 |
| | | (proline 4-hydroxylase), alpha II polypeptide | | | | | |
| | Pacrg | PARK2 co-regulated | 69310 | 0.5817 | 0.09 | 0.0000 | −1.16 |
| | Padi2 | peptidyl arginine deiminase, type II | 18600 | 0.0000 | 1.77 | 0.0000 | 1.63 |
| | Pafahlb3 | platelet-activating factor acetylhydrolase, isoform 1b, | 18476 | 0.5817 | 0.08 | 0.0046 | 0.49 |
| | | subunit 3 | | | | | |
| | Pamr1 | peptidase domain containing associated with muscle | 210622 | 0.7476 | −0.15 | 0.0046 | −0.68 |
| | | regeneration 1 | | | | | |
| | Pawr | PRKC, apoptosis, WTI, regulator | 114774 | 0.2806 | 0.19 | 0.0028 | −0.40 |
| | Pbk | PDZ binding kinase | 52033 | 0.1412 | −0.68 | 0.0028 | −0.86 |
| | Pccb | propionyl Coenzyme A carboxylase, beta polypeptide | 66904 | 0.5817 | 0.12 | 0.0028 | −0.44 |
| | Pcdh17 | protocadherin 17 | 219228 | 0.1056 | −0.47 | 0.0028 | −0.72 |
| | Pcdh18 | protocadherin 18 | 73173 | 0.4615 | 0.29 | 0.0089 | −0.56 |
| | Pcdhb17 | protocadherin beta 17 | 93888 | 0.0324 | −0.42 | 0.0046 | −0.63 |
| | Pcdhga5 | protocadherin gamma subfamily A, 5 | 93713 | 0.0781 | −0.56 | 0.0000 | −1.47 |
| | Pcdhgb5 | protocadherin gamma subfamily B, 5 | 93702 | 0.7658 | −0.08 | 0.0000 | −1.22 |
| | Pcdhgb6 | protocadherin gamma subfamily B, 6 | 93703 | 0.6857 | 0.05 | 0.0000 | −1.20 |
| | Pcp4l1 | Purkinje cell protein 4-like 1 | 66425 | 0.4615 | 0.22 | 0.0000 | −1.60 |
| | Pcyox1 | prenylcysteine oxidase 1 | 66881 | 0.4615 | 0.11 | 0.0089 | −0.39 |
| | Pde1c | phosphodiesterase 1C | 18575 | 0.0324 | −0.51 | 0.0046 | −0.33 |
| | Pde2a | phosphodiesterase 2A, cGMP-stimulated | 207728 | 0.2038 | 0.30 | 0.0046 | −0.43 |
| | Pde3a | phosphodiesterase 3A, cGMP inhibited | 54611 | 0.7658 | −0.08 | 0.0000 | −1.14 |
| | Pde5a | phosphodiesterase 5A, cGMP-specific | 242202 | 0.2038 | −0.52 | 0.0000 | −1.16 |
| | Pdgfrb | platelet derived growth factor receptor, beta | 18596 | 0.6857 | 0.14 | 0.0052 | −0.71 |
| | | polypeptide | | | | | |
| | Pdlim4 | PDZ and LIM domain 4 | 30794 | 0.0061 | 0.60 | 0.0019 | 0.76 |
| | Pef1 | penta-EF hand domain containing 1 | 67898 | 0.0000 | 0.63 | 0.1759 | 0.21 |
| | Pe nk | preproenkephalin | 18619 | 0.0000 | 1.38 | 0.0089 | 0.90 |
| | Pepd | peptidase D | 18924 | 0.0000 | 0.36 | 0.0000 | 0.65 |
| | Per1 | period homolog 1 (Drosophila) | 18626 | 0.0091 | −0.52 | 0.3561 | 0.15 |
| | Pfas | phosphoribosylformylglycinamidine synthase (FGAR | 237823 | 0.0000 | −0.91 | 0.5446 | 0.04 |
| | | amidotransferase) | | | | | |
| | Pfkp | phosphofructokinase, platelet | 56421 | 0.7658 | −0.05 | 0.0000 | −0.65 |
| | Pgap2 | post-GP1 attachment to proteins 2 | 233575 | 0.0000 | −1.27 | 0.0000 | −1.68 |
| | Pgf | placental growth factor | 18654 | 0.0091 | 0.59 | 0.0028 | −0.46 |
| | Pgm5 | phosphoglucomutase 5 | 226041 | 0.0147 | 0.44 | 0.0000 | 0.88 |
| | Phactr1 | phosphatase and actin regulator 1 | 218194 | 0.0000 | −0.76 | 0.0089 | −0.52 |
| | Phactr4 | phosphatase and actin regulator 4 | 100169 | 0.0000 | −0.48 | 0.3561 | −0.27 |
| | Pigr | polymeric immunoglobulin receptor | 18703 | 0.7476 | −0.09 | 0.0052 | −0.50 |
| | Pigu | phosphatidylinositol glycan anchor biosynthesis, class U | 228812 | 0.1056 | −0.32 | 0.0089 | −0.46 |
| | Pigyl | phosphatidylinositol glycan anchor biosynthesis, class | 66268 | 0.2038 | 0.21 | 0.0089 | 0.40 |
| | | Y-like | | | | | |
| | Pik3c2g | phosphatidylinositol 3-kinase, C2 domain containing, | 18705 | 0.0147 | −0.57 | 0.0000 | 0.84 |
| | | gamma polypeptide | | | | | |
| | Pip4k2a | phosphatidylinositol-5-phosphate 4-kinase, type II, | 18718 | 0.2038 | 0.18 | 0.0000 | −0.74 |
| | | alpha | | | | | |
| | Pip5k1b | phosphatidylinositol-4-phosphate 5-kinase, typa 1 beta | 18719 | 0.3708 | −0.24 | 0.0000 | −0.67 |
| | Pisd- | phosphatidylserine decarboxylase, pseudogene 1 | 236604 | 0.0324 | −0.63 | 0.0046 | −0.86 |
| | ps1 | | | | | | |
| | Pkia | protein kinase inhibitor, alpha | 18767 | 0.7658 | −0.01 | 0.0089 | −0.70 |
| | Pkn1 | protein kinase N1 | 320795 | 0.0061 | −0.55 | 0.2533 | −0.21 |
| | Pla2g2d | phospholipase A2, group IID | 18782 | 0.1056 | 0.43 | 0.0000 | −0.81 |
| | Pla2g2f | phospholipase A2, group IIF | 26971 | 0.0000 | 1.67 | 0.6046 | −0.07 |
| | Plau | plasminogen activator, urokinase | 18792 | 0.0781 | 0.36 | 0.0019 | 0.38 |
| | Plcg1 | phospholipase C, gamma 1 | 18803 | 0.0000 | −0.41 | 0.6046 | −0.01 |
| | Plcl2 | phospholipase C-like 2 | 224860 | 0.0000 | −0.41 | 0.3561 | −0.12 |
| | Plekhb1 | pleckstrin homology domain containing, family B | 27276 | 0.6857 | 0.05 | 0.0046 | −0.40 |
| | | (evectins) member 1 | | | | | |
| | Plekhh2 | pleckstrin homology domain containing, family H (with | 213556 | 0.0061 | −0.73 | 0.0089 | −0.50 |
| | | MyTH4 domain) member 2 | | | | | |
| | Plekhn1 | pleckstrin homology domain containing, family N | 231002 | 0.0091 | −0.61 | 0.2533 | 0.24 |
| | | member 1 | | | | | |
| | Plk1 | polo-like kinase 1 (Drosophila) | 18817 | 0.4615 | −0.33 | 0.0046 | −0.50 |
| | Poc1b | POC1 centriolar protein homolog B (Chlamydomonas) | 382406 | 0.0217 | −0.50 | 0.0028 | −0.62 |
| | Polr1b | polymerase (RNA) 1 polypeptide B | 20017 | 0.1056 | −0.30 | 0.0052 | −0.41 |
| | Polr2a | polymerase (RNA) II (DNA directed) polypeptide A | 20020 | 0.0061 | −0.76 | 0.5963 | −0.09 |
| | Pan2 | paraoxonase 2 | 330260 | 0.0147 | 0.33 | 0.0089 | 0.31 |
| | Pop4 | processing of precursor 4, ribonuclease P/MRP family, | 66161 | 0.7658 | −0.10 | 0.0000 | −1.29 |
| | | (S. cerevisiae) | | | | | |
| | Postn | periostin, osteoblast specific factor | 50706 | 0.0147 | 1.15 | 0.0052 | −0.61 |
| | Ppap2a | phosphatidic acid phosphatase type 2A | 19012 | 0.5817 | 0.24 | 0.0000 | −1.01 |
| | Pparg | peroxisome proliferator activated receptor gamma | 19016 | 0.1412 | −0.20 | 0.0028 | −0.43 |
| | Ppat | phosphoribosyl pyrophosphate amidotransferase | 231327 | 0.7569 | −0.08 | 0.0046 | −0.54 |
| | Pphln1 | periphilin 1 | 223828 | 0.7658 | −0.05 | 0.0089 | −0.54 |
| | Ppm1l | protein phosphatase 1 (formerly 2C)-like | 242083 | 0.7658 | −0.06 | 0.0089 | −0.49 |
| | Ppp1r3c | protein phosphatase 1, regulatory (inhibitor) subunit 3C | 53412 | 0.0000 | 0.37 | 0.0000 | −0.73 |
| | Ppp2r3a | protein phosphatase 2, regulatory subunit B″, alpha | 235542 | 0.0147 | −0.57 | 0.0000 | −0.80 |
| | Prcl | protein regulator of cytokinesis 1 | 233406 | 0.7187 | −0.35 | 0.0052 | −0.85 |
| | Prcp | prolylcarboxypeptidase (angiotensinase C) | 72461 | 0.4615 | 0.17 | 0.0089 | −0.44 |
| | Prex2 | phosphatidylinositol-3,4,5-trisphosphate-dependent | 109294 | 0.7187 | 0.02 | 0.0052 | −0.51 |
| | | Rac exchange factor 2 | | | | | |
| | Prkca | protein kinase C, alpha | 18750 | 0.0506 | −0.42 | 0.0000 | −0.78 |
| | Prkg1 | protein kinase, cGMP-dependent, type 1 | 19091 | 0.4615 | −0.27 | 0.0052 | −0.54 |
| | Prom1 | prominin 1 | 19126 | 0.2038 | −0.29 | 0.0000 | −1.56 |
| | Prox1 | prospera-related homeobox 1 | 19130 | 0.0000 | −0.60 | 0.5744 | −0.13 |
| | Prpf18 | PRP18 pre-mRNA processing factor 18 homolog (yeast) | 67229 | 0.0000 | 0.88 | 0.0046 | 0.68 |
| | Prss2 | protease, serine, 2 | 22072 | 0.2038 | 1.33 | 0.0019 | 1.03 |
| | Prss3 | protease, serine, 3 | 22073 | 0.1412 | 1.56 | 0.0089 | 1.20 |
| | Prune2 | prune homolog 2 (Drosophila) | 353211 | 0.6857 | 0.06 | 0.0052 | −0.57 |
| | Psg29 | pregnancy-specific glycoprotein 29 | 114872 | 0.7658 | −0.04 | 0.0019 | 0.46 |
| | Psmb2 | proteasome (prosome, macropain) subunit, beta type 2 | 26445 | 0.0091 | 0.49 | 0.2533 | 0.14 |
| | Psmb6 | proteasome (prosome, macropain) subunit, beta type 6 | 19175 | 0.0061 | 0.84 | 0.2533 | 0.19 |
| | Psmb8 | proteasome (prosome, macropain) subunit, beta type 8 | 16913 | 0.2038 | 0.22 | 0.0089 | 0.38 |
| | | (large multifunctional peptidase 7) | | | | | |
| | Psmc3ip | proteasome (prosome, macropain) 26S subunit, | 19183 | 0.6857 | 0.04 | 0.0019 | 0.61 |
| | | ATPase 3, interacting protein | | | | | |
| | Psme4 | proteasome (prosome, macropain) activator subunit 4 | 103554 | 0.0000 | −0.52 | 0.0052 | −0.40 |
| | Ptgr1 | prostaglandin reductase 1 | 67103 | 0.0091 | 0.59 | 0.0000 | 0.61 |
| | Ptp4al | protein tyrosine phosphatase 4al | 19243 | 0.0217 | −0.76 | 0.0052 | −0.89 |
| | Ptprj | protein tyrosine phosphatase, receptor type, J | 19271 | 0.0000 | −0.53 | 0.3561 | −0.18 |
| | Ptprr | protein tyrosine phosphatase, receptor type, R | 19279 | 0.2806 | −0.25 | 0.0000 | −0.66 |
| | Ptprs | protein tyrosine phosphatase, receptor type, S | 19280 | 0.4615 | −0.36 | 0.0052 | 0.58 |
| | Pttg1 | pituitary tumor-transforming gene 1 | 30939 | 0.0000 | −0.98 | 0.0000 | −0.71 |
| | Pvr | poliovirus receptor | 52118 | 0.2038 | 0.23 | 0.0046 | 0.44 |
| | Pyroxd2 | pyridine nucleotide-disulphide oxidoreductase domain 2 | 74580 | 0.3708 | −0.24 | 0.0019 | 0.64 |
| | Qsox1 | quiescin Q6 sulfhydryl oxidese 1 | 104009 | 0.0781 | 0.53 | 0.0046 | −0.65 |
| | R3hdm1 | R3H domain 1 (binds single-stranded nucleic acids) | 226412 | 0.0000 | −0.52 | 0.4909 | −0.19 |
| | Reb6b | RAB6B, member RAS oncogene family | 270192 | 0.1412 | −0.28 | 0.0000 | −0.77 |
| | Rap1gds1 | RAPI, GTP-GDP dissociation stimulator 1 | 229877 | 0.1412 | −0.25 | 0.0028 | −0.53 |
| | Rapgef4 | Rap guanine nucleotide exchange factor (GEF) 4 | 56508 | 0.1412 | −0.39 | 0.0028 | −0.69 |
| | Rarres1 | retinoic acid receptor responder (tazarotene induced) 1 | 109222 | 0.1412 | 0.46 | 0.0019 | 1.01 |
| | Rasgrf2 | RAS protein-specific guanine nucleotide-releasing | 19418 | 0.0091 | −0.55 | 0.5595 | −0.19 |
| | | factor 2 | | | | | |
| | Rassf8 | Ras association (RalGDS/AF-6) domain family (N- | 71323 | 0.7637 | −0.07 | 0.0052 | 0.29 |
| | | terminal) member 8 | | | | | |
| | Rbl2 | retinoblastoma-like 2 | 19651 | 0.1412 | −0.24 | 0.0052 | −0.29 |
| | Rbp7 | retinol binding protein 7, cellular | 63954 | 0.0000 | 1.34 | 0.0019 | 1.05 |
| | Rcc2 | regulator of chromosome condensation 2 | 108911 | 0.6857 | 0.04 | 0.0089 | −0.25 |
| | Rcn2 | reticulocalbin 2 | 26611 | 0.6857 | 0.03 | 0.0028 | −0.61 |
| | Rec8 | REC8 homolog (yeast) | 56739 | 0.7658 | −0.06 | 0.0000 | −1.27 |
| | Recql | RecQ protein-like | 19691 | 0.7187 | 0.02 | 0.0089 | −0.59 |
| | Reg1 | regenerating islet-derived 1 | 19692 | 0.2038 | 1.46 | 0.0000 | 1.01 |
| | Reln | reelin | 19699 | 0.7187 | −0.13 | 0.0052 | 0.46 |
| | Relt | RELT tumor necrosis factor receptor | 320100 | 0.7658 | −0.05 | 0.0089 | −0.34 |
| | Rgnef | Rho-guanine nucleotide exchange factor | 110596 | 0.0506 | −0.27 | 0.0046 | −0.42 |
| | Rgs4 | regulator of G-protein signaling 4 | 19736 | 0.2806 | 0.31 | 0.0019 | 0.72 |
| | Rgs7bp | regulator of G-protein signalling 7 binding protein | 52882 | 0.7187 | −0.11 | 0.0028 | −0.41 |
| | Rhbdl2 | rhomboid, veinlet-like 2 (Drosophila) | 230726 | 0.7658 | −0.01 | 0.0000 | −0.73 |
| | Rhox13 | reproductive homeobox 13 | 73814 | 0.4615 | 0.08 | 0.0089 | 0.23 |
| | Rimklb | ribosomal modification protein rimK-like family | 108653 | 0.7476 | −0.08 | 0.0046 | −0.43 |
| | | member B | | | | | |
| | Rims2 | regulating synaptic membrane exocytosis 2 | 116838 | 0.0091 | −0.64 | 0.4909 | −0.18 |
| | Riok3 | RIO kinase 3 (yeast) | 66878 | 0.0000 | −0.52 | 0.1759 | −0.16 |
| | Ripply3 | ripply3 homolog (zebrafish) | 170765 | 0.7658 | −0.02 | 0.0000 | −0.44 |
| | Rnf144a | ring finger protein 144A | 108089 | 0.5817 | 0.11 | 0.0052 | −0.48 |
| | Rnf150 | ring finger protein 150 | 330812 | 0.4615 | 0.13 | 0.0000 | −0.96 |
| | Rnf157 | ring finger protein 157 | 217340 | 0.0324 | −0.40 | 0.0052 | −0.31 |
| | Rnf186 | ring finger protein 186 | 66825 | 0.0061 | 0.94 | 0.4909 | 0.10 |
| | Rnf213 | ring finger protein 213 | 672511 | 0.0000 | −0.59 | 0.5963 | −0.09 |
| | Rnf5 | ring finger protein 5 | 54197 | 0.0091 | 0.41 | 0.2533 | 0.10 |
| | Rpl29 | ribosomal protein L29 | 19944 | 0.7187 | −0.19 | 0.0000 | −1.62 |
| | Rpl30 | ribosomal protein L30 | 19946 | 0.7187 | 0.03 | 0.0000 | −0.78 |
| | Rpp38 | ribonuclease P/MRP 38 subunit (human) | 227522 | 0.0000 | 0.97 | 0.0000 | 0.86 |
| | Rps2 | ribosomal protein S2 | 16898 | 0.4615 | 0.21 | 0.0000 | −1.41 |
| | Rrp8 | ribosomal RNA processing 8, methyltransferase, | 101867 | 0.0324 | −0.39 | 0.0089 | −0.43 |
| | | homolog (yeast) | | | | | |
| | Rsad2 | radical S-adenosyl methionine domain containing 2 | 58185 | 0.6857 | 0.05 | 0.0000 | −1.88 |
| | Rsph1 | radial spoke head 1 homolog (Chlamydomonas) | 22092 | 0.7187 | 0.02 | 0.0000 | 1.12 |
| | Rtkn2 | rhotekin 2 | 170799 | 0.6857 | 0.10 | 0.0000 | 1.90 |
| | Runx1t1 | runt-related transcription factor 1; translocated to, 1 | 12395 | 0.0324 | −0.32 | 0.0052 | −0.55 |
| | | (cyclin D-related) | | | | | |
| | S100a10 | S100 calcium binding protein A10 (calpactin) | 20194 | 0.0217 | 0.38 | 0.0000 | −0.59 |
| | S100a11 | S100 calcium binding protein A11 (calgizzarin) | 20195 | 0.4615 | 0.18 | 0.0028 | −0.68 |
| | S100a16 | S100 calcium binding protein A16 | 67860 | 0.0091 | 0.73 | 0.4909 | 0.08 |
| | S1pr1 | sphingosine-1-phosphate receptor 1 | 13609 | 0.7187 | 0.02 | 0.0089 | −0.42 |
| | Scaf4 | SR-related CTD-associated factor 4 | 224432 | 0.0091 | −0.48 | 0.5446 | 0.04 |
| | Scarb1 | scavenger receptor class B. member 1 | 20778 | 0.5817 | 0.17 | 0.0046 | −0.59 |
| | Scd2 | stearoyl-Coenzyme A desaturase 2 | 20250 | 0.6857 | 0.09 | 0.0000 | 0.78 |
| | Scg2 | secretogranin II | 20254 | 0.2038 | −0.25 | 0.0089 | −0.32 |
| | Scg5 | secretogranin V | 20394 | 0.7658 | −0.03 | 0.0000 | 0.55 |
| | Scn1b | sodium channel, voltage-gated, type 1, beta | 20266 | 0.7476 | −0.13 | 0.0019 | 0.74 |
| | Scnn1g | sodium channel, nonvoltage-gated 1 beta | 20277 | 0.5817 | −0.24 | 0.0028 | −0.61 |
| | Scnn1g | sodium channel, nonvoltage-gated 1 gamma | 20278 | 0.3708 | −0.27 | 0.0000 | −0.82 |
| | Scpep1 | serine carboxypeptidase 1 | 74617 | 0.5817 | 0.12 | 0.0019 | 0.58 |
| | Sdc4 | syndecan 4 | 20971 | 0.5817 | 0.07 | 0.0028 | −0.47 |
| | Sdpr | serum deprivation response | 20324 | 0.0000 | 0.88 | 0.0873 | 0.24 |
| | Sec24a | Sec24 related gene family, member A (S. cerevisiae) | 77371 | 0.0000 | −0.78 | 0.6046 | −0.03 |
| | Sel1l3 | sel-1 suppressor of lin-12-like 3 (C. elegans) | 231238 | 0.7476 | −0.13 | 0.0052 | −0.58 |
| | Sema7a | sema domain, immunoglobulin domain (Ig), and GPI | 20361 | 0.6857 | 0.05 | 0.0028 | −0.52 |
| | | membrane anchor, (semaphorin) 7A | | | | | |
| | Senp3 | SUMO/sentrin specific peptidase 3 | 80886 | 0.0000 | −0.48 | 0.5446 | −0.10 |
| | Serpina1b | serine (or cysteine) preptidase inhibitor, clada A, | 20701 | 0.0000 | −1.14 | 0.1158 | 0.59 |
| | | member 1B | | | | | |
| | Serpine 2 | serine (or cysteine) peptidase inhibitor, clade E, | 20720 | 0.7187 | −0.27 | 0.0000 | −1.01 |
| | | member 2 | | | | | |
| | Setd2 | SET domain containing 2 | 235626 | 0.0091 | −0.60 | 0.5595 | −0.17 |
| | Setd5 | SET domain containing 5 | 72895 | 0.0000 | −0.61 | 0.4909 | 0.06 |
| | Setdb1 | SET domain, bifurcated 1 | 84505 | 0.1412 | −0.16 | 0.0089 | −0.37 |
| | Sez6l | seizure related 6 homolog like | 56747 | 0.0506 | −0.44 | 0.0028 | −0.74 |
| | Sfrp5 | secreted frizzled-related sequence protein 5 | 54612 | 0.7658 | −0.10 | 0.0028 | −1.30 |
| | Sft2d2 | SFT2 domain containing 2 | 108735 | 0.2806 | 0.19 | 0.0028 | −0.49 |
| | Sgcd | sarcoglycan, delta (dystrophin-associated | 24052 | 0.7350 | −0.09 | 0.0028 | −0.47 |
| | | glycoprotein) | | | | | |
| | Sgk3 | serum/glucocorticoid regulated kinase 3 | 170755 | 0.7350 | −0.15 | 0.0046 | −0.70 |
| | Sh3bgrl3 | SH3 domain binding glutamic acid-rich protein-like 3 | 73723 | 0.0091 | 0.73 | 0.0046 | 0.49 |
| | Sh3pxd2a | SH3 and PX domains 2A | 14218 | 0.6857 | 0.07 | 0.0028 | −0.76 |
| | Sik1 | salt inducible kinase 1 | 17691 | 0.0000 | −0.66 | 0.1759 | 0.22 |
| | Siva1 | SIVA1, apoptosis-inducing factor | 30954 | 0.2038 | 0.29 | 0.0089 | 0.49 |
| | Six4 | sine oculis-related homeobox 4 homolog (Drosophila) | 20474 | 0.0324 | −0.50 | 0.0028 | −0.64 |
| | Slamf9 | SLAM family member 9 | 98365 | 0.7658 | −0.03 | 0.0052 | −0.46 |
| | Slc11a2 | solute carrier family 11 (proton-coupled divalent metal | 18174 | 0.7187 | 0.01 | 0.0019 | 0.44 |
| | | ion transporters), member 2 | | | | | |
| | Slc15a2 | solute carrier family 15 (H+/peptide transporter), | 57738 | 0.2038 | 0.30 | 0.0028 | −0.69 |
| | | member 2 | | | | | |
| | Slc18a1 | solute carrier family 18 (vesicular monoamine), | 110877 | 0.6857 | 0.07 | 0.0028 | −0.61 |
| | | member 1 | | | | | |
| | Slc20a2 | solute carrier family 20, member 2 | 20516 | 0.5817 | −0.18 | 0.0089 | −0.35 |
| | Slc22a23 | solute carrier family 22, member 23 | 73102 | 0.0506 | −0.55 | 0.0028 | −0.61 |
| | Slc25a15 | solute carrier family 25 (mitochondrial carrier | 18408 | 0.7658 | −0.04 | 0.0046 | −0.32 |
| | | ornithine transporter), member 15 | | | | | |
| | Slc26a1 | solute carrier family 26 (sulfate transporter), member 1 | 231583 | 0.7658 | −0.02 | 0.0000 | 1.07 |
| | Slc28a2 | solute carrier family 28 (sodium-coupled nucleoside | 269346 | 0.0000 | −0.93 | 0.0046 | −0.61 |
| | | transporter), member 2 | | | | | |
| | Slc29a1 | solute carrier family 29 (nucleoside transporters), | 63959 | 0.7658 | −0.08 | 0.0052 | −0.43 |
| | | member 1 | | | | | |
| | Slc37a1 | solute carrier family 37 (glycerol-3-phosphate | 224674 | 0.7637 | −0.10 | 0.0000 | 0.97 |
| | | transporter), member 1 | | | | | |
| | Slc38a11 | solute carrier family 38, member 11 | 320106 | 0.7658 | −0.05 | 0.0000 | −1.30 |
| | Slc39a8 | solute carrier family 39 (metal ion transporter), | 67547 | 0.7569 | −0.13 | 0.0089 | 0.73 |
| | | member 8 | | | | | |
| | Slc43a3 | solute carrier family 43, member 3 | 58207 | 0.1056 | 0.30 | 0.0046 | −0.46 |
| | Slc46a3 | solute carrier family 46, member 3 | 71706 | 0.7658 | −0.05 | 0.0052 | −0.44 |
| | Slc4a10 | solute carrier family 4, sodium bicarbonate | 94229 | 0.2806 | −0.26 | 0.0000 | −1.65 |
| | | cotransporter-like, member 10 | | | | | |
| | Slc5a1 | solute carrier family 5 (sodium/glucose | 20537 | 0.0091 | 0.44 | 0.3561 | 0.07 |
| | | cotransporter), member 1 | | | | | |
| | Slc7a8 | solute carrier family 7 (cationic amino acid | 50934 | 0.5817 | −0.15 | 0.0089 | −0.39 |
| | | transporterk, y+ system), member 8 | | | | | |
| | Slco1a5 | solute carrier organic anion transporter family, | 108096 | 0.6857 | 0.06 | 0.0028 | −0.58 |
| | | member 1a5 | | | | | |
| | Slco1a6 | solute carrier organic anion transporter family, | 28254 | 0.0091 | −0.92 | 0.0000 | 1.31 |
| | | member 1a6 | | | | | |
| | Slco3a1 | solute carrier organic anion transporter family, | 108116 | 0.6857 | 0.05 | 0.0028 | −0.37 |
| | | member 3a1 | | | | | |
| | Slit2 | slit homolog 2 (Drosophila) | 20563 | 0.7187 | 0.01 | 0.0089 | −0.42 |
| | Smg7 | Smg-7 homolog, nonsense mediated mRNA decay | 226517 | 0.0061 | −0.34 | 0.6046 | −0.03 |
| | | factor (C. elegans) | | | | | |
| | Snord104 | small nucleolar RNA, C/D box 104 | 100216537 | 0.2038 | 0.34 | 0.0089 | 0.35 |
| | Snord14e | small nucleolar RNA, C/D box 14E | 100302594 | 0.7350 | −0.42 | 0.0000 | 1.99 |
| | Snord32a | small nucleolar RNA, C/D box 32A | 27209 | 0.1056 | 0.45 | 0.0046 | 0.57 |
| | Snord34 | small nucleolar RNA, C/D box 34 | 27210 | 0.2038 | 0.34 | 0.0000 | 0.88 |
| | Snord35a | small nucleolar RNA, C/D box 35A | 27211 | 0.6857 | 0.05 | 0.0046 | 0.54 |
| | Snord49a | small nucleolar RNA, C/D box 49A | 100217455 | 0.4615 | 0.16 | 0.0000 | 0.66 |
| | Snord95 | small nucleolar RNA, C/D box 95 | 100216540 | 0.0091 | −0.76 | 0.3561 | 0.20 |
| | Snrnp27 | small nuclear ribonucleoprotein 27 (U4/U6.U5) | 66618 | 0.1056 | 0.38 | 0.0019 | 0.41 |
| | Sorll | sortilin-related receptor, LOLR class A repeats- | 20660 | 0.0506 | −0.51 | 0.0000 | 0.99 |
| | | containing | | | | | |
| | Sos1 | son of sevenless homolog 1 (Drosophila) | 20662 | 0.0000 | −0.66 | 0.3561 | −0.22 |
| | Sos2 | son of sevenless homolog 2 (Drosophila) | 20663 | 0.0061 | −0.37 | 0.3561 | 0.10 |
| | Sostdc1 | sclerostin domain containing 1 | 66042 | 0.0781 | 1.13 | 0.0089 | 1.12 |
| | Spaca1 | sperm acrosome associated 1 | 67652 | 0.5817 | 0.06 | 0.0052 | −0.35 |
| | Spag1 | sperm associated antigen 1 | 26942 | 0.6857 | 0.11 | 0.0000 | 1.18 |
| | Spc24 | SPC24, NDC80 kinetochore complex component, | 67629 | 0.4615 | 0.16 | 0.0052 | −0.36 |
| | | homolog (S. cerevisiae) | | | | | |
| | Spc25 | SPC25, NDC80 kinetochore complex component, | 66442 | 0.1412 | −0.49 | 0.0000 | −1.04 |
| | | homolog (S. cerevisiae) | | | | | |
| | Spg11 | spastic paraplegia 11 | 214585 | 0.0091 | −0.57 | 0.3561 | −0.25 |
| | Spink3 | serine peptidase inhibitor, Kazal type 3 | 20730 | 0.1412 | 2.03 | 0.0052 | 1.54 |
| | Spnb3 | spectrin beta 3 | 20743 | 0.0000 | −0.37 | 0.0089 | −0.34 |
| | Spock1 | sparc/osteonectin, CWCV and kazal-like domains | 20745 | 0.7658 | −0.02 | 0.0019 | 0.54 |
| | | proteoglycan 1 | | | | | |
| | Spock2 | sparc/asteonectin, CWCV and kazal-like domains | 94214 | 0.6857 | 0.05 | 0.0052 | 0.47 |
| | | proteoglycan 2 | | | | | |
| | Spon2 | spondin 2, extracellular matrix protein | 100689 | 0.7637 | −0.09 | 0.0089 | −0.39 |
| | Spred2 | sprouty-related, EVHI domain containing 2 | 114716 | 0.0000 | −0.61 | 0.0604 | −0.32 |
| | Spsb4 | splA/ryanodine receptor domain and SDCS box | 211949 | 0.7658 | −0.07 | 0.0052 | −0.37 |
| | | containing 4 | | | | | |
| | Srgn | serglycin | 19073 | 0.2038 | 0.23 | 0.0089 | −0.50 |
| | Srrm1 | serine/arginine repetitive matrix 1 | 51796 | 0.0061 | −0.52 | 0.5449 | −0.16 |
| | St3gal5 | ST3 beta-galactoside alpha-2,3-sialyltransferase 5 | 20454 | 0.5817 | 0.12 | 0.0089 | −0.43 |
| | St6gal2 | beta galactosida alpha 2,6 sialyltransferase 2 | 240119 | 0.7658 | −0.07 | 0.0089 | 0.43 |
| | Steap2 | six transmembrane epithelial antigen of prostate 2 | 74051 | 0.6857 | −0.22 | 0.0000 | −1.08 |
| | Steap4 | STEAP family member 4 | 117167 | 0.1412 | 0.30 | 0.0000 | −1.67 |
| | Stk10 | serine/threonine kinase, 10 | 20868 | 0.0000 | 0.46 | 0.0089 | 0.32 |
| | Ston1 | stonin 1 | 77057 | 0.7187 | −0.14 | 0.0028 | −0.65 |
| | Stox2 | storkhead box 2 | 71069 | 0.0000 | −0.56 | 0.5744 | −0.09 |
| | Stxbp6 | syntaxin binding protein 6 (amisyn) | 217517 | 0.1412 | −0.27 | 0.0000 | −0.67 |
| | Suv39h2 | suppressor of variegation 3-9 homolog 2 (Drosophila) | 64707 | 0.0506 | 0.41 | 0.0046 | 0.49 |
| | Synpr | synaptoporin | 72003 | 0.0217 | 0.51 | 0.0028 | −0.63 |
| | Syt9 | synaptotagmin IX | 60510 | 0.0000 | −0.83 | 0.0000 | −0.78 |
| | Sytl1 | synaptotagmin-like 1 | 269589 | 0.0061 | 0.63 | 0.0000 | 0.70 |
| | Taar1 | trace amine-associated receptor 1 | 111174 | 0.7187 | −0.24 | 0.0000 | −1.19 |
| | Taf4a | TAF4A RNA polymerase II, TATA box binding protein | 228980 | 0.0061 | −0.41 | 0.6046 | −0.07 |
| | | (TBP)-associated factor | | | | | |
| | Tat | tyrosine aminotransferase | 234724 | 0.2038 | −0.58 | 0.0028 | −0.71 |
| | TbcId22b | TBCI domain family, member 22B | 381085 | 0.0506 | −0.37 | 0.0089 | 0.56 |
| | TbcId8b | TBCI domain family member 8B | 245638 | 0.4615 | −0.31 | 0.0028 | −0.37 |
| | TbcId9 | TBCI domain family, member 9 | 71310 | 0.00781 | −0.39 | 0.0028 | −0.56 |
| | Tdp2 | tyrosyl-DNA phosphodiesterase 2 | 56196 | 0.6857 | 0.05 | 0.0089 | −0.58 |
| | Tert | telomerase reverse transcriptase | 21752 | 0.7350 | −0.10 | 0.0019 | 0.42 |
| | Tfrc | transferrin receptor | 22042 | 0.5817 | 0.14 | 0.0000 | −1.46 |
| | Tgfbr2 | transforming growth factor, beta receptor II | 21813 | 0.7187 | 0.03 | 0.0089 | −0.36 |
| | TgfbrapI | transforming growth factor, beta receptor associated | 73122 | 0.0000 | −0.45 | 0.5872 | −0.08 |
| | | protein I | | | | | |
| | Tgoln1 | trans-golgi network protein | 22134 | 0.7187 | 0.03 | 0.0028 | −0.56 |
| | Th | tyrosine hydroxylase | 21823 | 0.0506 | −0.62 | 0.0000 | −1.64 |
| | Thnsl2 | threonine synthase-like 2 (bacterial) | 232078 | 0.3708 | −0.14 | 0.0028 | −0.45 |
| | Thyn1 | thymocyte nuclear protein 1 | 77862 | 0.5817 | 0.15 | 0.0019 | 0.72 |
| | Tifa | TRAF-interacting protein with forkhead-associated | 211550 | 0.5817 | 0.10 | 0.0000 | 1.26 |
| | | domain | | | | | |
| | Tjap1 | tight junction associated protein 1 | 74094 | 0.7658 | 0.00 | 0.0028 | −0.48 |
| | Tlcd2 | TLC domain containing 2 | 380712 | 0.4615 | 0.14 | 0.0019 | 0.62 |
| | Tmc7 | transmembrane channal-like gene family 7 | 209760 | 0.0061 | 0.68 | 0.4909 | −0.17 |
| | Tmcc3 | transmembrane and coiled coil domains 3 | 319880 | 0.1412 | −0.26 | 0.0046 | −0.52 |
| | Tmem130 | transmembrane protein 130 | 243339 | 0.6857 | 0.07 | 10.0000 | 1.26 |
| | Tmem131 | transmembrane protein 131 | 56030 | 0.0000 | −0.73 | 0.3561 | −0.22 |
| | Tmem45a | transmembrane protein 45a | 56277 | 0.0000 | 0.90 | 0.0000 | 1.76 |
| | Tmem86b | transmembrane protein 86B | 68255 | 0.0000 | −0.64 | 0.5446 | 0.05 |
| | Tmad1 | trapomodulin 1 | 21916 | 0.7658 | −0.06 | 0.0028 | −0.87 |
| | Tmprss2 | transmembrane protease, serine 2 | 50528 | 0.7658 | −0.04 | 0.0000 | −0.60 |
| | Tmprss4 | transmembrane protease, serine 4 | 214523 | 0.7658 | −0.05 | 0.0089 | 0.46 |
| | Tmtc3 | transmembrane and tetratricopeptide repeat | 237500 | 0.1412 | −0.27 | 0.0028 | −0.51 |
| | | containing 3 | | | | | |
| | Tmub2 | transmembrane and ubiquitin-like domain containing 2 | 72053 | 0.6857 | 0.07 | 0.0000 | −0.35 |
| | Tnfaip2 | tumor necrosis factor, alpha-induced protein 2 | 21928 | 0.7187 | 0.04 | 0.0052 | −0.74 |
| | Tnfaip8 | tumor necrosis factor, alpha-induced protein 8 | 106869 | 0.5817 | 0.23 | 0.0028 | −0.92 |
| | Tnfrsf21 | tumor necrosis factor receptor superfamily, member | 94185 | 0.7187 | 0.01 | 0.0028 | −0.58 |
| | | 21 | | | | | |
| | Top2a | topoisomerase (DNA) II alpha | 21973 | 0.4615 | −0.54 | 0.0028 | −0.85 |
| | Tpx2 | TPX2, microtubule-associated protein homolog | 72119 | 0.4615 | −0.41 | 0.0089 | −0.52 |
| | | (Xenopus laevis) | | | | | |
| | Trac | T cell receptor alpha constant | 100101484 | 0.0091 | 1.60 | 0.2533 | 0.56 |
| | Trf | transferrin | 22041 | 0.4615 | 0.20 | 0.0000 | −0.55 |
| | Trim12a | tripartite motif-containing 12A | 76681 | 0.0000 | −1.77 | 0.0000 | −2.44 |
| | Trim12c | tripartite motif-containing 12C | 319236 | 0.6857 | −1.20 | 0.0089 | −0.67 |
| | Trio | triple functional domain (PTPRF interacting) | 223435 | 0.0000 | −0.35 | 0.5446 | 0.03 |
| | TrmtII2 | tRNA methyltransferase II-2 homolog (S. cerevisiae) | 67674 | 0.0091 | 0.42 | 0.0387 | 0.36 |
| | Trnp1 | TMFI-regulated nuclear protein 1 | 69539 | 0.0000 | 1.16 | 0.0000 | 1.31 |
| | Trpc3 | transient receptor potential cation channel, subfamily | 22065 | 0.6857 | 0.04 | 0.0000 | −0.55 |
| | | C, member 3 | | | | | |
| | Trpc4 | transient receptor potential cation channel, subfamily | 22066 | 0.1056 | −0.28 | 0.0052 | −0.29 |
| | | C, member 4 | | | | | |
| | Trrap | transformation/transcription domain-associated | 100683 | 0.0061 | −0.57 | 0.5446 | 0.01 |
| | | protein | | | | | |
| | Tshz3 | teashirt zinc finger family member 3 | 243931 | 0.0147 | −0.34 | 0.0052 | −0.51 |
| | Tspan8 | tetraspanin 8 | 216350 | 0.7658 | −0.14 | 0.0028 | −1.07 |
| | Ttc30b | tetratricopeptide repeat domain 30B | 72421 | 0.7350 | −0.16 | 0.0028 | −0.85 |
| | Ttr | transthyretin | 22139 | 0.2038 | −0.32 | 0.0028 | −0.57 |
| | Tufm | Tu translation elongation factor, mitochondrial | 233870 | 0.0000 | −0.77 | 0.0046 | −0.81 |
| | Tulp4 | tubby like protein 4 | 68842 | 0.0000 | −0.50 | 0.1759 | −0.21 |
| | Txlna | taxilin alpha | 109658 | 0.0061 | −0.54 | 0.0261 | −0.34 |
| | UapIl1 | UDP-N-acteylglucosamine pyrophosphorylase I-like 1 | 227620 | 0.4615 | 0.29 | 0.0000 | 0.69 |
| | Ube2d2 | ubiquitin-conjugating enzyme E2D 2 | 56550 | 0.0061 | −0.48 | 0.0261 | −0.34 |
| | UblcpI | ubiquitin-like domain containing CTD phosphatase I | 79560 | 0.0147 | −0.81 | 0.0000 | −1.09 |
| | Ubr5 | ubiquitin protein ligase E3 component n-recognin 5 | 70790 | 0.0061 | −0.51 | 0.2533 | −0.18 |
| | Uchl1 | ubiquitin carboxy-terminal hydrolase L1 | 22223 | 0.1412 | 0.56 | 0.0052 | −0.57 |
| | Ulk4 | unc-5I-like kinase 4 (C. elegans) | 209012 | 0.1412 | −0.27 | 0.0000 | −0.72 |
| | Uox | urate oxidase | 22262 | 0.6857 | 0.03 | 0.0000 | −0.71 |
| | Upbl | ureidopropionase, beta | 103149 | 0.3708 | 0.33 | 0.0028 | −0.98 |
| | Ush2a | Usher syndrome 2A (autosomal recessive, mild) | 22283 | 0.0061 | −0.61 | 0.0387 | −0.30 |
| | | homolog (human) | | | | | |
| | Usp34 | ubiquitin specific peptidase 34 | 17847 | 0.0091 | −0.63 | 0.5446 | −0.17 |
| | Uxt | ubiquitously expressed transcript | 22294 | 0.6857 | 0.08 | 0.0089 | 0.87 |
| | Vil1 | villin 1 | 22349 | 0.0506 | −0.50 | 0.0000 | −1.08 |
| | Vldlr | very low density lipoprotein receptor | 22359 | 0.0217 | −0.42 | 0.0046 | −0.36 |
| | VmnIr90 | vomeronasal I receptor 90 | 627280 | 0.2038 | −0.37 | 0.0000 | −0.93 |
| | Vpsl3d | vacuolar protein sorting 13 D (yeast) | 230895 | 0.0000 | −1.13 | 0.0028 | −0.64 |
| | Vrk2 | vaccinia related kinase 2 | 69922 | 0.2038 | −0.20 | 0.0000 | −0.58 |
| | Vsnl1 | visinin-like 1 | 26950 | 0.4615 | 0.22 | 0.0019 | 0.77 |
| | Vtn | vitronectin | 22370 | 0.0324 | 1.74 | 0.0019 | 1.62 |
| | Wdfy1 | WD repeat and FYVE domain containing 1 | 69368 | 0.6857 | 0.08 | 0.0000 | 0.94 |
| | Wdfy3 | WD repeat and FYVE domain containing 3 | 72145 | 0.0000 | −0.52 | 0.5595 | −0.15 |
| | Wdr18 | WD repeat domain 18 | 216156 | 0.0091 | 0.51 | 0.3561 | 0.06 |
| | Wdr49 | WD repeat domain 49 | 213248 | 0.6857 | 0.12 | 0.0046 | 0.99 |
| | Wdyhv1 | WDYHV motif containing 1 | 76773 | 0.0781 | 0.26 | 0.0089 | 0.41 |
| | Wee1 | WEE 1 homolog 1 (S. pombe) | 22390 | 0.0091 | −0.61 | 0.0000 | −0.82 |
| | Wfdc10 | WAP four-disulfide core domain 10 | 629756 | 0.4615 | 0.12 | 0.0089 | 0.53 |
| | WnkI | WNK lysine deficient protein kinase I | 232341 | 0.0091 | −0.50 | 0.5595 | −0.13 |
| | Wnk3- | WNK lysine deficient protein kinase 3, pseudogene | 279561 | 0.0147 | −0.73 | 0.0028 | −0.89 |
| | ps | | | | | | |
| | Wrap53 | WD repeat containing, antisense to TP53 | 216853 | 0.0000 | 0.59 | 0.5963 | −0.06 |
| | Wtap | Wilms' tumour I-associating protein | 60532 | 0.0781 | −0.37 | 0.0000 | 0.85 |
| | Xpo6 | exportin 6 | 74204 | 0.0091 | −0.42 | 0.6046 | −0.04 |
| | Xrcc6 | X-ray repair complementing defective repair in | 14375 | 0.4615 | 0.14 | 0.0000 | −0.96 |
| | | Chinese hamster cells 6 | | | | | |
| | Zadh2 | zinc binding alcohol dehydrogenase, domain containing 2 | 225791 | 0.1056 | −0.38 | 0.0089 | −0.53 |
| | Zbtb40 | zinc finger and BTB domain containing 40 | 230848 | 0.0091 | −0.54 | 0.2533 | −0.21 |
| | Zfp14 | zinc finger protein 14 | 243906 | 0.5817 | 0.07 | 0.0000 | 0.64 |
| | Zfp318 | zinc finger protein 318 | 57908 | 0.0000 | −0.58 | 0.5446 | 0.01 |
| | Zfp365 | zinc finger protein 365 | 216049 | 0.6857 | −0.15 | 0.0000 | 0.65 |
| | Zfp566 | zinc finger protein 566 | 72556 | 0.1056 | 0.50 | 0.0000 | 0.96 |
| | Zfp61 | zinc finger protein 61 | 22719 | 0.7350 | −0.10 | 0.0052 | 0.52 |
| | Zfp619 | zinc finger protein 619 | 70227 | 0.7187 | 0.02 | 0.0089 | −0.25 |
| | Zfp637 | zinc finger protein 637 | 232337 | 0.0091 | 0.41 | 0.3561 | 0.11 |
| | Zfp791 | zinc finger protein 791 | 244556 | 0.4615 | 0.17 | 0.0052 | −0.47 |
| | Zfp87 | zinc finger protein 87 | 170763 | 0.7658 | −0.02 | 0.0052 | −0.75 |
| | Zfp931 | zinc finger protein 931 | 353208 | 0.0091 | 1.25 | 0.5595 | −0.33 |
| | Zfr | zinc finger RNA binding protein | 22763 | 0.2038 | −0.23 | 0.0028 | −0.49 |
| | Znrd1 | zinc ribbon domain containing, 1 | 66136 | 0.0061 | 0.43 | 0.3561 | −0.12 |
| | Zwilch | Zwilch, kinetochore associated, homolog (Drosophila) | 68014 | 0.6857 | −0.22 | 0.0089 | −0.61 |
| | Zzef1 | zinc finger, ZZ-type with EF hand domain 1 | 195018 | 0.0000 | −0.60 | 0.6046 | −0.01 |

TABLE 2
MEDLINE CITATION COUNTS (OUTPUT)

| Gene | Gene | | | NOD vs. | | NOD vs. | | TOTAL | “diabetes” | COUNT |
|---|---|---|---|---|---|---|---|---|---|---|
| symbol | title | Gene Title | GeneID | NOR | | C57BI/6 | | COUNT | COUNT | RATIO |
| sdf | Abhd10 | abhydrolase domain containing 10 | 213012 | 0.3708 | 0.14 | 0.0028 | −0.51 | 5586 | 216 | 0 |
| fasd | Abcd2 | ATP-binding cassette, sub-family | 26874 | 0.6857 | 0.11 | 0.0052 | −0.90 | 4534 | 64 | 0 |
| | | D (ALD), member 2 | | | | | | | | |
| fasd | Adarb2 | adenosine deaminase, RNA- | 94191 | 0.3708 | −0.15 | 0.0089 | 0.32 | 4296 | 22 | 0 |
| | | specific, B2 | | | | | | | | |
| fasd | Acsm3 | acyl-CoA synthetase medium- | 20216 | 0.0000 | −1.38 | 0.4909 | 0.04 | 4291 | 21 | 0.035714286 |
| | | chain family member 3 | | | | | | | | |
| fasd | Abhd1 | abhydrolase domain containing 1 | 57742 | 0.2806 | 0.17 | 0.0052 | 0.67 | 4280 | 21 | 0.033333333 |
| asdf | Acp1 | acid phosphatase 1, soluble | 11431 | 0.2806 | 0.30 | 0.0028 | −0.98 | 414 | 30 | 0.070175439 |
| asdf | Abca3 | ATP-binding cassette, sub-family | 27410 | 0.0000 | −0.46 | 0.3561 | 0.08 | 297 | 2 | 0.147540984 |
| | | A (ABCI), member 3 | | | | | | | | |
| asdf | Abcb1a | ATP-binding cassette, sub-family | 18671 | 0.2038 | 0.24 | 0.0000 | −0.76 | 169 | 5 | 0.044117647 |
| | | B (MDR/TAP), member 1A | | | | | | | | |
| asdf | Aard | alanine and arginine rich domain | 239435 | 0.7187 | −0.18 | 0.0028 | −0.63 | 79 | 2 | 0.025316456 |
| | | containing protein | | | | | | | | |
| asdf | Adam22 | a disintegrin and | 11496 | 0.2806 | −0.34 | 0.0052 | −0.73 | 79 | 1 | 0.012658228 |
| | | metallopeptidase domain 22 | | | | | | | | |
| asdf | Acss2 | acyl-CoA synthetase short-chain | 60525 | 0.7187 | −0.12 | 0.0000 | −0.73 | 68 | 3 | 0.029585799 |
| | | family member 2 | | | | | | | | |
| asdf | Acad1 | acyl-Coenzyme A dehydrogenase, | 11363 | 0.5817 | 0.17 | 0.0028 | −1.01 | 61 | 9 | 0.006734007 |
| | | long-chain | | | | | | | | |
| asdf | Acsl6 | acyl-CoA synthetase long-chain | 216739 | 0.0000 | −0.68 | 0.0000 | −0.59 | 57 | 4 | 0.072463768 |
| | | family member 6 | | | | | | | | |
| asdf | Acot13 | acyl-CoA thioesterase 13 | 66834 | 0.1412 | 0.45 | 0.0028 | −0.78 | 30 | 1 | 0.004906542 |
| asdf | Acad8 | acyl-Coenzyme A dehydrogenase | 66948 | 0.0217 | 0.32 | 0.0046 | 0.41 | 28 | 1 | 0.004893964 |
| | | family, member 8 | | | | | | | | |
| asdf | Acss3 | acyl-CoA synthetase short-chain | 380660 | 0.6857 | 0.07 | 0.0089 | 0.55 | 20 | 0 | 0.005121043 |
| | | family member 3 | | | | | | | | |
| asdf | Abhd14b | abhydrolase domain containing | 76491 | 0.2038 | 0.32 | 0.0000 | −0.67 | 12 | 0 | 0.014115571 |
| | | 14b | | | | | | | | |
| asdf | AA388235 | expressed sequence AA388235 | 433100 | 0.5817 | 0.10 | 0.0000 | −1.33 | 11 | 0 | 0.038668099 |

What is claimed is:

1. A method of data mining based on microarray data database and a document database, comprising:
receiving microarray data;
generating a first search of a microarray data database for information for interpreting the microarray data;
determining sequences of interest of the microarray data based on results of the first search;
receiving a topical annotation;
generating a second set of searches of a document database for documents corresponding to the sequences of interest, and a conjunction of the sequences of interest and the annotation;
performing at least one comparative quantitative analysis between a first quantity of citations of the document database for documents corresponding to the sequences of interest versus a second quantity of citations for documents corresponding to a conjunction of the sequences of interest and the annotation; and
ranking the sequences of interest based on the comparative quantitative analysis.

2. The method according to claim 1, wherein a sequence of interest having a high ratio of the first quantity of citations to the second quantity of citations ranks higher than a sequence of interest having a low ratio of the first quantity of citations to the second quantity of citations.

3. The method according to claim 1, further comprising presenting the ranking based on the comparative quantitative analysis as a word cloud.

4. The method according to claim 1, wherein the microarray data database comprises the NCBI GEO database.

5. The method according to claim 1, wherein the document database comprises the NCBI Pubmed database.

6. The method according to claim 1, wherein the microarray data database is accessed through the Internet.

7. The method according to claim 1, wherein the document database is accessed through the Internet.

8. The method according to claim 1, further comprising excluding sequences of interest for which the first quantity of references is below a threshold number from the ranking.

9. A system for data mining based on microarray data database and a document database, comprising:
an input port configured to receive microarray data; a communication network interface port;
at least one processor, configured to:
generate a first search of a microarray data database for information for interpreting the microarray data;
conduct the first search on the microarray data database through the communication network interface port;
determine sequences of interest of the microarray data based on results of the first search;
receive a topical annotation;
generate a second set of searches for a document database for documents corresponding to the sequences of interest, and a conjunction of the sequences of interest and the annotation;
conduct the second search on the document data database through the communication network interface port;
perform at least one comparative quantitative analysis between a first quantity of citations of the document database for documents corresponding to the sequences of interest versus a second quantity of citations for documents corresponding to a conjunction of the sequences of interest and the annotation; and
rank the sequences of interest based on the comparative quantitative analysis; and
an output port configured to present the ranked sequences.

10. The system according to claim 9, wherein a sequence of interest having a high ratio of the first quantity of citations to the second quantity of citations is ranked higher than a sequence of interest having a low ratio of the first quantity of citations to the second quantity of citations.

11. The system according to claim 9, wherein ranked sequences comprise a word cloud.

12. The system according to claim 9, wherein the microarray data database comprises the NCBI GEO database.

13. The system according to claim 9, wherein the document database comprises the NCBI Pubmed database.

14. The system according to claim 9, wherein the communication network interface port comprises an Internet interface.

15. The system according to claim 9, wherein the at least one processor is further configured to exclude sequences of interest for which the first quantity of references is below a threshold number.

16. A computer readable medium storing thereon nontransitory instructions for causing an automated data processing system to perform the steps of:
generating a first search of a microarray data database for information for interpreting a set of microarray data;
conducting the first search on the microarray data database through a communication network interface;
determining sequences of interest of the microarray data based on results of the first search;
receiving a topical annotation;
generating a second set of searches for a document database for documents corresponding to the sequences of interest, and a conjunction of the sequences of interest and the annotation;
conducting the second search on the document data database through the communication network interface;
performing at least one comparative quantitative analysis between a first quantity of citations of the document database for documents corresponding to the sequences of interest versus a second quantity of citations for documents corresponding to a conjunction of the sequences of interest and the annotation; and
ranking the sequences of interest based on the comparative quantitative analysis.

17. The computer readable medium according to claim 16, wherein a sequence of interest having a high ratio of the first quantity of citations to the second quantity of citations ranks higher than a sequence of interest having a low ratio of the first quantity of citations to the second quantity of citations.

18. The computer readable medium according to claim 16, further comprising nontransitory instructions presenting the ranking based on the comparative quantitative analysis as a word cloud.

19. The computer readable medium according to claim 16, wherein the microarray data database comprises the NCBI GEO database.

20. The computer readable medium according to claim 16, wherein sequences of interest for which the first quantity of references is below a threshold number are excluded from the ranking.
